(19) United States
(12) Patent Application Publication
(10) Pub. No.: US 2024/0415951 A1
(43) Pub. Date: Dec. 19, 2024

(54) LENTIVIRAL VECTOR FOR EXPRESSION OF HUMAN PAPILLOMAVIRUS (HPV) ANTIGENS AND ITS IMPLEMENTATION IN THE TREATMENT OF HPV INDUCED CANCERS

(71) Applicants: THERAVECTYS, Paris (FR); INSTITUT PASTEUR, Paris (FR)

(72) Inventors: Pierre CHARNEAU, Paris (FR); François ANNA, Paris (FR); Fanny MONCOQ, Paris (FR); Amandine NOIRAT, Paris (FR); Laleh MAJLESSI, Paris (FR); Laetitia DOUGUET, Paris (FR); Ingrid FERT, Paris (FR)

(21) Appl. No.: 18/706,610

(22) PCT Filed: Nov. 14, 2022
(86) PCT No.: PCT/EP2022/081839

§ 371 Date: May 1, 2024

Related U.S. Application Data
(60) Provisional application No. 63/279,945, filed on Nov. 16, 2021.

(30) Foreign Application Priority Data
Nov. 15, 2021 (EP) 21306581.6
Jul. 27, 2022 (EP) 22306119.3

(51) Int. Cl. A61K 39/12; A61K 39/00; A61P 35/00; C07K 14/005; C12N 15/86

(52) U.S. Cl. CPC A61K 39/12; A61P 35/00; C07K 14/005; C12N 15/86; A61K 2039/5256; C12N 2710/20022; C12N 2710/20034; C12N 2740/15043; C12N 2830/48

(57) ABSTRACT

The present invention relates to a lentiviral vector, in particular a non-integrative lentiviral vector, comprising at least four distinct nucleic acid sequences encoding HPV antigens, to a lentiviral vector particle comprising said vector, to an isolated cell comprising the same, and to a vaccine composition comprising the said lentiviral vector, lentiviral vector particle or cell. The present invention further relates to their implementation in the treatment or prevention of an HPV induced cancer.

### FIELD OF THE INVENTION

The present invention relates to the field of recombinant vaccine technology and relates to improvements of lentiviral vectors, which can be used as therapeutic and prophylactic vaccines. In particular, the present invention relates to lentiviral vectors expressing Human Papillomavirus (HPV) antigens and to their implementation in the prevention and treatment of HPV induced cancers.

### BACKGROUND OF THE INVENTION

HPV is responsible for 5.2% of cancers throughout the world (Tota et al., Prev Med 2011 October; 53 Suppl 1:S12-21). More than 100 HPV types have been identified and have been classified into 3 groups depending on their association with cancer: high risk-types with a high oncogenic potential (among them, HPV type 16 (HPV-16) and HPV type 18 (HPV-18)), low risk-types that are associated with benign lesions (HPV-6, -11) and cutaneous types (among them, HPV-1, -2, -3, -4 . . . ) (Chen et al. Virology vol. 516 (2018): 86-101). The fraction of HPV associated cancer varies upon cancer type and geography, but it is estimated that 90% of cervical, 91% of anal, 75% of vaginal, 70% of oropharyngeal, 69% of vulvar and 63% of penile cancers are related with HPV infections (Saraiya et al., J Natl Cancer Inst. 2015 Apr. 29; 107(6)).

The two most common types of HPV found in cancers are HPV16 and HPV18. For instance, HPV16 and HPV18 are thought to be involved in 70-75% of all cervical cancers (de Sanjose et al., Eur J Cancer. 2013 November; 49(16):3450-61). HPV16 and 18 are also largely involved in anal (91%), oropharyngeal (70%), vaginal (75%), penile (63%) and vulvar (68%) cancers (Saraiya et al., J Natl Cancer Inst. 2015 Apr. 29; 107(6)).

Thereby, a therapeutic vaccine targeting HPV16/18 could potentially be used to treat and prevent related cancers, irrespective of their location.

Human papillomaviruses (HPV) are non-enveloped, double-stranded DNA viruses. Their genome encodes six non-structural proteins (early proteins E1, E2, E4, E5, E6 and E7) and two structural proteins (late proteins L1 and L2) (Chen et al. Virology vol. 516 (2018): 86-101).

Among theses proteins, E6 and E7 have been well characterized for their oncogenicity. They are known to inactivate p53 and pRb tumour suppressor proteins, thereby promoting cell proliferation. E6/E7 oncogenes are crucial for both the induction of HPV-linked malignant cell transformation and the maintenance of the oncogenic phenotype of HPV-positive cancer cells (Yim and Park, Cancer Res Treat. 2005 December; 37(6):319-24.). The E6 and E7 proteins are expressed throughout the infection by all HPV positive cells and this observation makes them perfect targets for vaccines.

Recombinant viral vectors have been widely developed for vaccination purposes. Modification of viral genomes allowed the production of non-toxic and non-infectious viral particles that can be used as tools to introduce genetic material into target cells. The use of recombinant viral vectors to elicit a T-cell mediated immunity is a very promising approach for vaccination. A variety of viral vectors have been evaluated for vaccination purposes including retroviruses vectors, adenoviruses vectors and vaccinia virus vectors (Milone and O'Doherty, Leukemia (2018) 32:1529-1541 and Ku et al., Expert Review of Vaccines (2021). Lentiviruses are part of the Retroviridae family, which includes the human immunodeficiency viruses (HIV). Lentiviral vectors are mainly derived from HIV-1. They have been improved in their safety by removal of the LTR U3 sequence, resulting in “self-inactivating” vectors that are entirely devoid of viral promoter and enhancer sequences. Lentiviral vectors have emerged as promising tools because they exhibit several advantages over other viral systems. In particular, lentiviral vectors are not toxic and, unlike other retroviruses, are capable of transducing non-dividing cells, in particular dendritic cells (He et al. 2007, Expert Rev vaccines, 6(6):913-24), allowing a sustained antigen presentation through the endogenous pathway.

As opposed to the other commonly used viral vectors, lentiviral vectors have the capacity to transduce non-dividing cells. Efficient transduction in non-dividing cells requires the formation of a triple-stranded DNA structure called the central DNA “flap”, which maximizes the efficiency of gene import into the nuclei of non-dividing cells, including dendritic cells (DCs) (Arhel et al., EMBO J. 2007 Jun. 20; 26(12): 3025-3037) (Zennou et al., Cell. 2000 Apr. 14; 101(2):173-85).

Dendritic cells (DCs) are of primary importance for antigen presentation as they constitute the main class of antigen presenting cells (APCs) whose primary function is to present antigens and initiate an immune response (Steinman, R., Banchereau, J. Nature 449, 419-426 (2007)). Mature DCs migrate to the draining lymph node and where they present the antigen-derived short peptides at the surface through Major Histocompatibility Complex (MHC) molecules. Antigen-specific T cells present in the lymph nodes can then interact with peptide-MHC complexes through TCR (T Cell Receptor). The recognition of peptide-MHC by specific TCR in conjunction with co-stimulatory signals, initiates T cell activation (Steinman, R., Banchereau, J. Nature 449, 419-426 (2007)).

The aim of the present invention is to provide therapeutic and prophylactic vaccines for the prevention and treatment of HPV-induced cancers.

Therapeutic vaccination against high-risk human papillomaviruses with an integrase defective lentiviral vector expressing non-oncogenic HPV16 E7 fused to calreticulin (CRT) has been described (Grasso et al., Int J Cancer. 2013 Jan. 15; 132(2):335-44). The tests were performed against early-stage tumors and showed the ability of this construct to eradicate said tumors in a reasonable but not perfect number of vaccinated mice.

Accordingly, there is still a need in the art for the treatment of more aggressive and/or well implanted tumors induced by HPV, which are known in the art as being more difficult to eliminate than small and early-stage tumors.

There is also a need for a treatment of resistant tumors induced by HPV, i.e. tumors characterized in that they are strongly infiltrated with Regulatory T cells (Tregs).

There is moreover a need for a therapeutic vaccine allowing infiltration with CD8⁺ and CD4⁺ cells in HPV-induced tumors to be treated while reducing Tregs in said tumors.

There is also a need for the generation of a strong immunological memory against HPV and in particular against PDHPV antigens, more particularly against HPV16 and HPV18 antigens.

There is also a need for the generation of a novel safe, non-oncogenic, prophylactic and therapeutic vaccine against HPV induced cancers.

There is further a need for a vaccine allowing, after administration of a single dose, to fully eliminate primary tumors and to provide a strong protection against relapse.

The invention has for purpose to meet the above-mentioned needs.

### SUMMARY OF THE INVENTION

The present invention accordingly relates to the following items:

Item 1: a lentiviral vector, in particular a non-integrative lentiviral vector, comprising at least four distinct nucleic acid sequences selected from the group consisting of:
at least one nucleic acid sequence encoding a non-oncogenic Human papillomavirus (HPV16) protein E6 antigen,
at least one nucleic acid sequence encoding a non-oncogenic Human papillomavirus (HPV16) protein E7 antigen,
at least one nucleic acid sequence encoding a non-oncogenic Human papillomavirus (HPV18) protein E6 antigen, and
at least one nucleic acid sequence encoding a non-oncogenic Human papillomavirus (HPV18) protein E7 antigen.

As illustrated in the examples, lentiviral vectors of the invention allow for a strong therapeutic and prophylactic activity against HPV-induced tumors.

Item 2: The lentiviral vector according to item 1, wherein the nucleic acid sequence encoding the non-oncogenic Human papillomavirus (HPV16) protein E6 antigen encodes an amino acid sequence having at least 80% sequence identity with the amino acid sequence set forth as SEQ ID NO: 7, the nucleic acid sequence being in particular selected from the group consisting of SEQ ID NO: 2, SEQ ID NO: 3, SEQ ID NO: 4, SEQ ID NO: 5 and SEQ ID NO: 6.

Item 3: The lentiviral vector according to item 1 or 2, wherein the nucleic acid sequence encoding the non-oncogenic Human papillomavirus (HPV16) protein E7 antigen encodes an amino acid sequence having at least 68% sequence identity with the amino acid sequence set forth as SEQ ID NO: 16, the nucleic acid sequence being in particular selected from the group consisting of SEQ ID NO: 14 and SEQ ID NO: 15.

Item 4: The lentiviral vector according to any one of items 1 to 3, wherein the nucleic acid sequence encoding the non-oncogenic Human papillomavirus (HPV18) protein E6 antigen encodes an amino acid sequence having at least 60% sequence identity with the amino acid sequence set forth as SEQ ID NO: 24, the nucleic acid sequence being in particular selected from the group consisting of SEQ ID NO: 20, SEQ ID NO: 21, SEQ ID NO: 22 and SEQ ID NO: 23.

Item 5: The lentiviral vector according to any one of items 1 to 4, wherein the nucleic acid sequence encoding the non-oncogenic Human papillomavirus (HPV18) protein E7 antigen encodes an amino acid sequence having at least 83% sequence identity with the amino acid sequence set forth as SEQ ID NO: 33, the nucleic acid sequence being in particular selected from the group consisting of SEQ ID NO: 30, SEQ ID NO: 31 and SEQ ID NO: 32.

Item 6: The lentiviral vector according to any one of items 1 to 5, wherein the at least four distinct nucleic acid sequences encoding antigens are fused together, forming a single antigenic nucleic acid sequence encoding a single antigenic fusion protein under the control of a single promoter sequence.

Item 7: The lentiviral vector according to any one of items 1 to 6, wherein the order of the at least four distinct nucleic acid sequences, from 5′ end to 3′ end, is selected from the group consisting of:
(a) nucleic acid sequence encoding a non-oncogenic Human papillomavirus (HPV16) protein E7 antigen—nucleic acid sequence encoding a non-oncogenic Human papillomavirus (HPV16) protein E6 antigen—nucleic acid sequence encoding a non-oncogenic Human papillomavirus (HPV18) protein E7 antigen—nucleic acid sequence encoding a non-oncogenic Human papillomavirus (HPV18) protein E6 antigen;
(b) nucleic acid sequence encoding a non-oncogenic Human papillomavirus (HPV16) protein E6 antigen—nucleic acid sequence encoding a non-oncogenic Human papillomavirus (HPV18) protein E6 antigen—nucleic acid sequence encoding a non-oncogenic Human papillomavirus (HPV16) protein E7 antigen—nucleic acid sequence encoding a non-oncogenic Human papillomavirus (HPV18) protein E7 antigen;
(c) nucleic acid sequence encoding a non-oncogenic Human papillomavirus (HPV18) protein E6 antigen—nucleic acid sequence encoding a non-oncogenic Human papillomavirus (HPV16) protein E6 antigen—nucleic acid sequence encoding a non-oncogenic Human papillomavirus (HPV18) protein E7 antigen—nucleic acid sequence encoding a non-oncogenic Human papillomavirus (HPV16) protein E7 antigen; and
(d) nucleic acid sequence encoding a non-oncogenic Human papillomavirus (HPV18) protein E7 antigen—nucleic acid sequence encoding a non-oncogenic Human papillomavirus (HPV18) protein E6 antigen—nucleic acid sequence encoding a non-oncogenic Human papillomavirus (HPV16) protein E7 antigen—nucleic acid sequence encoding a non-oncogenic Human papillomavirus (HPV16) protein E6 antigen.

Item 8: The lentiviral vector according to any one of items 1 to 7, wherein the order of the at least four distinct nucleic acid sequences, from 5′ end to 3′ end, is (a) nucleic acid sequence encoding a non-oncogenic Human papillomavirus (HPV16) protein E7 antigen—nucleic acid sequence encoding a non-oncogenic Human papillomavirus (HPV16) protein E6 antigen—nucleic acid sequence encoding a non-oncogenic Human papillomavirus (HPV18) protein E7 antigen—nucleic acid sequence encoding a non-oncogenic Human papillomavirus (HPV18) protein E6 antigen.

Item 9: The lentiviral vector according to any one of items 1 to 8, comprising a nucleic acid sequence which encodes an amino acid sequence having at least 90% sequence identity with the amino acid sequence set forth as SEQ ID NO: 42, the nucleic acid sequence being in particular the nucleic acid sequence SEQ ID NO: 41.

Item 10: The lentiviral vector according to any one of items 1 to 9, being selected from the group consisting of the non-integrative lentiviral vectors filed at the CNCM under accession numbers I-5759, I-5760, I-5761 and I-5762, and is in particular the non-integrative lentiviral vector filed at the CNCM under accession number I-5759.

Item 11: The lentiviral vector according to any one of items 1 to 10, wherein the lentiviral vector comprises a MHC Class I promoter, and in particular a β2-microglobulin promoter.

Item 12: The lentiviral vector according to any one of items 1 to 11, wherein the lentiviral vector comprises a cPPT/CTS sequence, in particular the cPPT/CTS sequence set forth as sequence SEQ ID NO: 37.

Item 13: The lentiviral vector according to any one of items 1 to 12, wherein the lentiviral vector comprises a 3′ long terminal repeat (LTR) which is devoid of its U3 promoter sequence.

Item 14: The lentiviral vector according to any one of items 1 to 13, wherein the lentiviral vector does not comprise a constitutive enhancer sequence.

Item 15: The non-integrative lentiviral vector according to any one of items 1 to 14, wherein the lentiviral vector comprises a mutant form of the woodchuck hepatitis B virus (WHV) post-transcriptional regulatory element (WPRE), and in particular having the sequence set forth as sequence SEQ ID NO: 38.

Item 16: A lentiviral vector particle, in particular a non-integrative lentiviral vector particle, comprising at least one lentiviral vector as defined in any one of items 1 to 15.

Item 17: The lentiviral vector particle according to item 16, wherein the lentiviral vector particle comprises a functional lentiviral integrase protein.

Item 18: The lentiviral vector particle according to item 16 or 17, wherein the lentiviral vector particle comprises a vesicular stomatitis virus glycoprotein (VSVG), in particular a VSV-G Indiana serotype or a VSV-G New Jersey serotype.

Item 19: The lentiviral vector particle according to any one of items 16 to 18, wherein the lentiviral vector particle comprises HIV-1 subtype D Gag and Pol proteins.

Item 20: An isolated cell comprising the lentiviral vector according to any one of items 1 to 14 or the lentiviral vector particle according to any of items 16 to 19.

Item 21: A vaccine composition comprising a lentiviral vector according to any one of items 1 to 14, a lentiviral vector particle according to any one of items 16 to 19, or a cell according to item 19.

Item 22: The vaccine composition according to item 21, for use in the treatment or prevention of an HPV induced cancer, in particular selected from the group consisting of cervical cancer, vaginal cancer, vulvar cancer, penile cancer, anal cancer, oropharyngeal cancer, and metastases thereof, in particular pulmonary metastasis thereof.

Item 23: A lentiviral vector according to any one of items 1 to 15, a lentiviral vector particle according to any one of items 16 to 19, or a cell according to item 20, for use as a medicament or vaccine.

Item 24: The lentiviral vector, lentiviral vector particle or cell, according to item 23, for use in the treatment or prevention of an HPV induced cancer, in particular selected from the group consisting of cervical cancer, vaginal cancer, vulvar cancer, penile cancer, anal cancer, oropharyngeal cancer, and metastases thereof, in particular pulmonary metastasis thereof.

Item 25: The vaccine composition for use according to item 22, or the lentiviral vector, lentiviral vector particle or cell for use according to item 23 or 24, wherein said vaccine composition, lentiviral vector, lentiviral vector particle or cell is administered in combination with at least one immune checkpoint inhibitor, in particular at least one monoclonal antibody selected from the group consisting of anti-PD-1, anti-PD-L1, anti-CTLA-4, anti-NKG2A, anti-TIM-3, anti-TIGIT and anti-LAG-3 monoclonal antibodies and more particularly with at least one anti-PD-1 monoclonal antibody.

Item 26: The vaccine composition, lentiviral vector, lentiviral vector particle or cell for use according to item 25, wherein the at least one immune checkpoint inhibitor is administered simultaneously or separately, and in particular the at least one immune checkpoint inhibitor is administered at least 2, and in particular at least 4 days after the administration of the said vaccine composition, lentiviral vector, lentiviral vector particle or cell.

The details, examples and preferences provided in relation to any particular one or more of the stated aspects of the present invention will be further described herein and apply equally to all aspects of the present invention. Any combination of the embodiments, examples and preferences described herein in all possible variations thereof is encompassed by the present invention unless otherwise indicated herein, or otherwise clearly contradicted by context.

### BRIEF DESCRIPTION OF THE FIGURES

FIG. 1 demonstrates that HPV vaccines of the invention are immunogenic in vivo. Mice were injected i.m. with 1×10⁷ TU of lentiviral vector particles comprising the lentiviral vector filed at the CNCM under accession number I-5759, the lentiviral vector filed at the CNCM under accession number I-5760, the lentiviral vector filed at the CNCM under accession number I-5761 or the lentiviral vector filed at the CNCM under accession number I-5762 or 50 μL of diluent. 14 days later, splenocytes were prepared and restimulated overnight for IFNg ELISPOT with 4 distinct peptide pools.

Abscissa: from left to right: results obtained with the lentiviral vector filed at the CNCM under accession number I-5759, the lentiviral vector filed at the CNCM under accession number I-5760, the lentiviral vector filed at the CNCM under accession number I-5761, the lentiviral vector filed at the CNCM under accession number I-5762 or with 50 μL of diluent (control).

Ordinate: Spot Forming Cells (SFC)/10⁶ cells.

FIG. 2 shows that invention vaccine fully eliminates well implanted tumors in vivo. TC-1 cells were injected s.c. and tumor volume was measured every other day (caliper measurement). When average tumor volume is 70 mm³, mice were randomized and vaccinated with 1×10⁸ TU i.m. of LV-GFP Indiana (as a control), Indiana lentiviral vector particles comprising T-5759, Indiana lentiviral vector particles comprising T-5760, Indiana lentiviral vector particles comprising I-5761 or Indiana lentiviral vector particles comprising I-5762.

Abscissa: Days.

Ordinate: Tumor volume (mm³).

FIG. 3 illustrates the ability of lentiviral vector according to the invention expressing design I-5759, I-5760, I-5761, I-5762, or diluent as control, to generate a long lasting immunity to prevent relapses. Mice which eliminated primary tumor were rechallenged on the other flank at day 60. Control mice (untreated) were also injected s.c. in order to check on the tumor cell growth in naïve mice.

Abscissa: Days.

Ordinate: Tumor volume (mm³).

FIG. 4 represents a dose/response in mouse. 1×10⁶ TC-1 cells were injected in the flank of animals and tumor volume was measured twice a week (caliper measurement). When average tumor volume was 80 mm³, mice were randomized and vaccinated with diluent (control), 1×10⁷ TU of I-5759 vaccine or 1×10⁸ TU (i.m.) of I-5759 vaccine.

Abscissa: Days.

Ordinate: Tumor volume (mm³).

FIG. 5 represents lymphocytic tumor infiltration after vaccination with a lentiviral vector according to the invention. 1×10⁶ TC1 tumor cells were injected (s.c.) on the flank of animal, and the tumor volume was measured twice a week (caliper measurement). When average tumor volume was 80 mm³, mice were randomized and vaccinated with either diluent (control), or 1×10⁷ TU of I-5759 vaccine or 1×10⁸ TU (i.m.) of I-5759 vaccine.

Ten days after vaccination, tumors were collected, digested and analyzed by flow cytometry. FACS staining was performed and data were acquired on Macsquant facs according to methods well known in the art.

Abscissa: from left to right: diluent (control); 1×10⁸ TU of T-5759 vaccine.

Ordinate: Top left Figure: % CD8+ T cells (within live cells); Top right Figure: % CD4+ T cells (within live cells); Bottom Figure: % Treg cells (within live cells).

FIG. 6 represents the ability of vectors according to the invention to eliminate well-established large tumors. 1×10⁶ TC1 cells were injected (s.c.) on the flank of animal. When average tumor volume was around 300 mm³, mice were randomized and vaccinated with diluent (control), or 1×10⁸ TU (i.m.) of the vaccine according to the invention comprising lentiviral vector particles comprising the lentiviral vector filed at the CNCM under accession number I-5759.

Abscissa: Days.

Ordinate: Tumor volume (mm³).

FIG. 7 depicts T cell response in human PBMC labelled with CFSE and cultured in absence (unstimulated condition) or presence of a vaccine according to the invention (I-5759). Cell proliferation and activation were measured after 2 weeks of culture (n=3). CD8+ T cells and CD4+ T cells proliferation (measured by CFSE dilution) (A) and expression of CD25 activation marker (B) were increased by addition of lentiviral vectors of the invention in the culture.

Abscissa: from left to right: Unstimulated (Unstim—control); I-5759 vaccine.

Ordinate: (A) Left Figure: % of CFSElow in CD8+ population; Right Figure: % of CFSElow in CD4+ population. (B) Left Figure: % of CD25+ in CD4+ population; Right Figure: % of CD25+ in CD8+ population.

FIGS. 8A to 8D depicts four examples of antigen constructs of lentiviral vectors according to the invention. Each of these antigen constructs consists in the four following sequences in various orders: a nucleic acid sequence encoding a non-oncogenic Human papillomavirus (HPV16) protein E6 antigen, a nucleic acid sequence encoding a non-oncogenic Human papillomavirus (HPV16) protein E7 antigen, a nucleic acid sequence encoding a non-oncogenic Human papillomavirus (HPV18) protein E6 antigen and a nucleic acid sequence encoding a non-oncogenic Human papillomavirus (HPV18) protein E7 antigen. FIG. 8A represents the antigen construct of the lentiviral vector filed at the CNCM under accession number I-5759. FIG. 8B represents the antigen construct of the lentiviral vector filed at the CNCM under accession number T-5760. FIG. 8C represents the antigen construct of the lentiviral vector filed at the CNCM under accession number I-5761 and FIG. 8D represents the antigen construct of the lentiviral vector filed at the CNCM under accession number I-5762.

FIGS. 9A and 9B depict T-cell cytokine responses of splenocytes analyzed by Intracellular Cytokine Staining (ICS), with or without stimulation with a mixture of ETTDPDRAHYNIVTF (SEQ ID NO: 39) and PDRAHYNIVTFCCKC (SEQ ID NO: 40) synthetic peptides, both containing the RAHYNIVTF H-2D^{b}-restricted T-cell epitope (bold characters represent H-2D^{b} anchor residues) (SEQ ID NO: 49), the splenocytes being obtained 14 days post-vaccination of C57BL/6 mice (n=5/group) through i.m. immunization with a Ctrl Lenti (LV-GFP Indiana) or with a vaccine according to the invention comprising lentiviral vector particles comprising the lentiviral vector filed at the CNCM under accession number I-5759. FIG. 9A in particular represents the cytometric gating strategy carried out on cytokine-producing CD8+ T cells and degranulation activity of the IFN-γ-producing CD8⁺ T cells, assessed by surface CD107a staining. FIG. 9B represents the recapitulative frequencies of each (poly)functional cell subsets and of IFN-γ⁺ CD107a⁺ cells within the CD8⁺ T subset.

FIG. 10 depicts cytometric analysis of tumor infiltrating innate immune cells (NK) of tumor-engrafted and vaccinated with a HPV-vaccine according to the invention (I-5759), or of tumor-engrafted and non vaccinated mice (control—Ctrl Lenti). CD11b and NKp46 were detected.

FIG. 11 represents the ability of vectors according to the invention to eliminate well-established large tumors. A rechallenge of cured mice which eliminated the primary tumor (right panel) was performed on the other flank with 1·10⁶ TC-1 tumor cells 119 days after the first engraftment and were maintained without any therapy. Control mice (untreated—control aged-mice—left panel) were also injected s.c. in order to check on the tumor cell growth.

Abscissa: Days after tumor rechallenge.

Ordinate: Tumor volume (mm³).

FIGS. 12A and 12B illustrate the synergy between a vaccination with a suboptimal dose of a vaccine according to the invention (I-5759) and an anti-PD-1 therapy (monoclonal antibody anti-PD-1).

FIG. 12A represents the evolution of tumor volumes (mm3—ordinate) over days post tumor engraftement in mice (D0) (abscissa). Experiments were performed on three identical groups of tumor-engrafted mice. In the first group (10 mice—group control—left panel of FIG. 12A), the mice were administered a LV-empty Indiana (D13) (as a control) (day indicated by an arrow) and four days later with an anti-PD-1 (Programmed cell Death protein-1) monoclonal antibody (D17, then D20, D22, D24, D28 and D31). In the second group (12 mice—group control—middle panel of FIG. 12A), the mice were administered a vaccine according to the invention (I-5759) (D13) (day indicated by an arrow) and four days later with a control antibody (isotype ctrl) (D17, then D20, D22, D24, D28 and D31). In the third group (14 mice—left panel of FIG. 12A), the mice were administered a vaccine according to the invention (D13) (I-5759) (day indicated by an arrow) and four days later with a mAb anti-PD-1 (D17, then D20, D22, D24, D28 and D31).

FIG. 12B represents the survival (% of mice—ordinate) of the mice of each group (group control 1: Ctrl Lenti+anti-PD-1; group control 2: I-5759+ctrl Ig; group 3: I-5759+anti-PD-1) over time (days—abscissa).

FIGS. 13A and 13B represent the cure of mice with pulmonary metastatic foci induced by intravenous injection of TC1-nLuc cells after a single infection of Lenti-HPV-07 vaccine.

FIG. 13A represents the variation of luminescence values expressed as photons per second (p/s) (ordinate—Total Flux) due to nanoluciferase stably expressed by TC1-nLuc cells injected to different groups of mice overtime (abscissa—days) after i.v. injection of said cells to said mice. 3 groups of mice were tested: a negative control group with mice not being injected with TC1-nLuc cells [symbol] (n=11) (Neg Ctrl), a control group with mice injected with TC1-nLuc cells and at day 5 with a Control Lenti (LV-empty Indiana)—1·10⁹ TU (n=11) (TC1+Ctrl Lenti) [symbol] and a group with mice injected with TC1-nLuc cells and at day 5 with a vaccine according to the invention (Lenti-HPV-07 (I-5759))—1·10⁹ TU (n=11) (TC1+Lenti-HPV-07) [symbol] .

FIG. 13B represents the individual p/s values, for individual mice of the three experimental groups detailed above, at day 22 post tumor injection. Ordinate: luminescence values expressed as photons per second (p/s) (Total Flux). Abscissa, from left to right: group Neg Ctrl, group Ctrl Lenti then group Lenti-HPV-07.

### SUMMARY OF THE SEQUENCES

SEQ ID NO: 1 is a nucleic acid sequence encoding the E6 protein from HPV 16

SEQ ID NO: 2 is a nucleic acid sequence encoding a non-oncogenic variant of the E6 protein from HPV 16

SEQ ID NO: 3 is a nucleic acid sequence encoding a non-oncogenic variant of the E6 protein from HPV 16

SEQ ID NO: 4 is a nucleic acid sequence encoding a non-oncogenic variant of the E6 protein from HPV 16

SEQ ID NO: 5 is a nucleic acid sequence encoding a non-oncogenic variant of the E6 protein from HPV 16

SEQ ID NO: 6 is a nucleic acid sequence encoding a non-oncogenic variant of the E6 protein from HPV 16

SEQ ID NO: 7 is the amino acid sequence of the E6 protein from HPV 16

SEQ ID NO: 8 is an amino acid sequence of a non-oncogenic variant of the E6 protein from HPV 16

SEQ ID NO: 9 is an amino acid sequence of a non-oncogenic variant of the E6 protein from HPV 16

SEQ ID NO: 10 is an amino acid sequence of a non-oncogenic variant of the E6 protein from HPV 16

SEQ ID NO: 11 is an amino acid sequence of a non-oncogenic variant of the E6 protein from HPV 16

SEQ ID NO: 12 is an amino acid sequence of a non-oncogenic variant of the E6 protein from HPV 16

SEQ ID NO: 13 is a nucleic acid sequence encoding the E7 protein from HPV 16

SEQ ID NO: 14 is a nucleic acid sequence encoding a non-oncogenic variant of the E7 protein from HPV 16

SEQ ID NO: 15 is a nucleic acid sequence encoding a non-oncogenic variant of the E7 protein from HPV 16

SEQ ID NO: 16 is the amino acid sequence of the E7 protein from HPV 16

SEQ ID NO: 17 is an amino acid sequence of a non-oncogenic variant of the E7 protein from HPV 16

SEQ ID NO: 18 is an amino acid sequence of a non-oncogenic variant of the E7 protein from HPV 16

SEQ ID NO: 19 is a nucleic acid sequence encoding the E6 protein from HPV 18

SEQ ID NO: 20 is a nucleic acid sequence encoding a non-oncogenic variant of the E6 protein from HPV 18

SEQ ID NO: 21 is a nucleic acid sequence encoding a non-oncogenic variant of the E6 protein from HPV 18

SEQ ID NO: 22 is a nucleic acid sequence encoding a non-oncogenic variant of the E6 protein from HPV 18

SEQ ID NO: 23 is a nucleic acid sequence encoding a non-oncogenic variant of the E6 protein from HPV 18

SEQ ID NO: 24 is the amino acid sequence of the E6 protein from HPV 18

SEQ ID NO: 25 is an amino acid sequence of a non-oncogenic variant of the E6 protein from HPV 18

SEQ ID NO: 26 is an amino acid sequence of a non-oncogenic variant of the E6 protein from HPV 18

SEQ ID NO: 27 is an amino acid sequence of a non-oncogenic variant of the E6 protein from HPV 18

SEQ ID NO: 28 is an amino acid sequence of a non-oncogenic variant of the E6 protein from HPV 18

SEQ ID NO: 29 is a nucleic acid sequence encoding the E7 protein from HPV 18

SEQ ID NO: 30 is a nucleic acid sequence encoding a non-oncogenic variant of the E7 protein from HPV 18

SEQ ID NO: 31 is a nucleic acid sequence encoding a non-oncogenic variant of the E7 protein from HPV 18

SEQ ID NO: 32 is a nucleic acid sequence encoding a non-oncogenic variant of the E7 protein from HPV 18

SEQ ID NO: 33 is the amino acid sequence of the E7 protein from HPV 18

SEQ ID NO: 34 is an amino acid sequence of a non-oncogenic variant of the E7 protein from HPV 18

SEQ ID NO: 35 is an amino acid sequence of a non-oncogenic variant of the E7 protein from HPV 18

SEQ ID NO: 36 is an amino acid sequence of a non-oncogenic variant of the E7 protein from HPV 18

SEQ ID NO: 37 is a nucleic acid sequence encoding the cPPT/CTS sequence

SEQ ID NO: 38 is the nucleic acid sequence encoding a mutant form of the woodchuck hepatitis B virus (WHV) post-transcriptional regulatory element (WPRE)

SEQ ID NO: 39 is a synthetic E7_{HPV16}-derived peptide containing the RAHYNIVTF H-2D^{b}-restricted T-cell epitope

SEQ ID NO: 40 is a synthetic E7_{HPV16}-derived peptide containing the RAHYNIVTF H-2D^{b}-restricted T-cell epitope

### DETAILED DESCRIPTION OF THE INVENTION

The inventors have discovered that the administration of a lentiviral vector encoding at least four distinct human papillomavirus (HPV) antigens, and in particular at least 4 HPV antigens selected among proteins E6 and E7 of at least two different HPV subtypes, and in particular of HPV16 and HPV18 subtypes, to an individual in need thereof results in a high prophylactic and therapeutic activity on HPV induced cancers.

Lentiviral vectors according to the invention can enable the induction of a strong, lasting and broad cell-mediated response against tumors induced by an HPV infection.

Lentiviral vectors according to the invention, as well as lenviral vector particules comprising them, isolated cells comprising said lentiviral vectors or lentiviral vector particles and vaccine compositions comprising them are described throughout the present specification.

### Definitions

All scientific and technical terms used in this application have meanings commonly used in the art unless otherwise specified.

As used herein, “transgene” means a polynucleotide that can be expressed, via recombinant techniques, in a non-native environment or heterologous cell under appropriate conditions.

As used herein, the term “recombinant”, when used in reference to a cell of the invention, indicates that the cell has been modified by the introduction of an endogenous and/or heterologous nucleic acid or protein into the cell or the alteration of a native cell or that the cell is derived from a cell so modified. Thus, for example, recombinant cells express genes or nucleic acid that are not found within the native (non-recombinant) form of the cell or express native (eg endogenous) genes at a different level than their native level or express additional or supplementary copies of native (eg endogenous) at a different level than their native level. An isolated cell according to the invention is recombinant in that it comprises at least one lentiviral vector according to the invention and/or at least one lentiviral vector particle according to the invention.

As used herein, the term “recombinant”, when used in reference to a vector, are sequences formed/obtained by technics of genetic engineering well known to the man skilled in the art.

As used herein, the term “polypeptide” refers to a molecule comprising amino acid residues linked by peptide bonds and containing more than five amino acid residues. The amino acids are identified by either the single-letter or three-letter designations. The term “protein” as used herein is synonymous with the term “polypeptide” and may also refer to two or more polypeptides. Thus, the terms “protein”, “peptide” and “polypeptide” can be used interchangeably. Polypeptides may optionally be modified (e.g., glycosylated, phosphorylated, acylated, farnesylated, prenylated, sulfonated, and the like) to add functionality. Polypeptides exhibiting activity may be referred to as enzymes. It will be understood that, as a result of the degeneracy of the genetic code, a multitude of nucleotide sequences encoding a given polypeptide may be produced.

The term “operably linked” as used herein refers to two or more nucleic acid sequence elements that are physically linked and are in a functional relationship with each other. For instance, in a lentiviral vector according to the invention, a promoter is operably linked to a coding sequence, also termed herein “antigen construct” as the promoter is able to initiate or regulate the transcription or expression of the antigen construct, in which case the antigen construct should be understood as being “under the control of” the promoter. Generally, when two nucleic acid sequences are operably linked, they will be in the same orientation and usually also in the same reading frame. They usually will be essentially contiguous, although this may not be required.

The terms “encoding” or “coding for” refer to the process by which a polynucleotide, through the mechanisms of transcription and translation, produces an amino-acid sequence.

For each or the amino acid sequences of interest, reference sequences are described herein. The present description also encompasses amino acid sequences having specific percentages of amino acid identity with a reference amino acid sequence.

For obvious reasons, in all the present description, a specific nucleic acid sequence or a specific amino acid sequence which complies with, respectively, the considered nucleotide or amino acid identity, should further lead to obtaining a protein (or antigen) which displays the desired biological activity. As used herein, the “percentage of identity” between two nucleic acid sequences or between two amino acid sequences is determined by comparing both optimally aligned sequences through a comparison window.

The portion of the nucleotide or amino-acid sequence in the comparison window may thus include additions or deletions (for example “gaps”) as compared to the reference sequence (which does not include these additions or these deletions) so as to obtain an optimal alignment between both sequences.

The terms “sequence homology” or “sequence identity” or “homology” or “identity” are used interchangeably herein. For the purpose of the invention, it is defined here that in order to determine the percentage of sequence homology or sequence identity of two amino acid sequences or of two nucleic acid sequences, the sequences are aligned for optimal comparison purposes. In order to optimize the alignment between the two sequences gaps may be introduced in any of the two sequences that are compared. Such alignment can be carried out over the full length of the sequences being compared. Alternatively, the alignment may be carried out over a shorter length, for example over about 20, about 50, about 100 or more nucleic acids/based or amino acids. The sequence identity is the percentage of identical matches between the two sequences over the reported aligned region.

A comparison of sequences and determination of percentage of sequence identity between two sequences can be accomplished using a mathematical algorithm. The skilled person will be aware of the fact that several different computer programs are available to align two sequences and determine the identity between two sequences (Kruskal, J. B. (1983) An overview of sequence comparison In D. Sankoff and J. B. Kruskal, (ed.), Time warps, string edits and macromolecules: the theory and practice of sequence comparison, pp. 1-44 Addison Wesley).

The percent sequence identity between two amino acid sequences or between two nucleotide sequences may be determined using the Needleman and Wunsch algorithm for the alignment of two sequences. (Needleman, S. B. and Wunsch, C. D. (1970) J. Mol. Biol. 48, 443-453). Both amino acid sequences and nucleotide sequences can be aligned by the algorithm. The Needleman-Wunsch algorithm has been implemented in the computer program NEEDLE.

For the purpose of the invention, the NEEDLE program from the EMBOSS package was used (version 2.8.0 or higher, EMBOSS: The European Molecular Biology Open Software Suite (2000) Rice, P. Longden J. and Bleasby, A. Trends in Genetics 16, (6) pp 276-277, http://emboss.bioinfonnatics.nl/). For protein sequences EBLOSUM62 is used for the substitution matrix. For nucleotide sequence, EDNAFULL is used. The optional parameters used are a gap opening penalty of 10 and a gap extension penalty of 0.5. No end gap penalty is added. In the Output section, Yes has been indicated in response to the question “Brief identity and similarity” and “SRS pairwise” indicated as Output alignment format.

After alignment by the program NEEDLE as described above the percentage of sequence identity between a query sequence and a sequence of the invention is calculated as follows: Number of corresponding positions in the alignment showing an identical amino acid or identical nucleotide in both sequences divided by the total length of the alignment after subtraction of the total number of gaps in the alignment. The identity defined as herein can be obtained from NEEDLE by using the NOBRIEF option and is labeled in the output of the program as “longest-identity”.

The similarity of nucleotide and amino acid sequences, i.e. the percentage of sequence identity, can be determined via sequence alignments using several other art-known algorithms, preferably with the mathematical algorithm of Karlin and Altschul (Karlin & Altschul (1993) Proc. Natl. Acad. Sci. USA 90: 5873-5877), with hmmalign (HMMER package, http://hmmer.wustl.edu/) or with the CLUSTAL algorithm (Thompson, J. D., Higgins, D. G. & Gibson, T. J. (1994) Nucleic Acids Res. 22, 4673-80) available e.g. on https://www.ebi.ac.uk/Tools/msa/clustalo/or the GAP program (mathematical algorithm of the University of Iowa) or the mathematical algorithm of Myers and Miller (1989—Cabios 4: 11-17) or Clone Manager 9. Preferred parameters used are the default parameters as they are set on https://www.ebi.ac.uk/Tools/msa/clustalo/.

The grade of sequence identity (sequence matching) may be calculated using e.g. BLAST, BLAT or BlastZ (or BlastX). A similar algorithm is incorporated into the BLASTN and BLASTP programs of Altschul et al (1990) J. Mol. Biol. 215, 403-410. BLAST polynucleotide searches are performed with the BLASTN program, score=100, word length=12, to obtain polynucleotide sequences that are homologous to those nucleic acids which encode the relevant protein.

BLAST protein searches are performed with the BLASTP program, score=50, word length=3, to obtain amino acid sequences homologous to the SHC polypeptide. To obtain gapped alignments for comparative purposes, Gapped BLAST is utilized as described in Altschul et al (1997) Nucleic Acids Res. 25, 3389-3402. When utilizing BLAST and Gapped BLAST programs, the default parameters of the respective programs are used. Sequence matching analysis may be supplemented by established homology mapping techniques like Shuffle-LAGAN (Brudno M., Bioinformatics 2003b, 19 Suppl 1: 154-162) or Markov random fields. When percentages of sequence identity are referred to in the present application, these percentages are calculated in relation to the full length of the longer sequence, if not specifically indicated otherwise.

In particular embodiments, % identity between two sequences is determined using CLUSTAL O (version 1.2.4).

The terms non-oncogenic used herein are used in their traditional meaning, i.e. it relates to an element, in the present case to antigens, unable to cause the formation of tumors. As detailed elsewhere, antigens implemented in the present invention have been genetically amended in order to become nononcogenic. It means, according to the usual meaning of these terms, that the nucleic acid sequences encoding the antigens implemented herein are not found in nature and are modified either by introduction or by deletion or by modification of their nucleic acid sequences, leading to encoded amino acid sequences that also do not naturally exist in nature.

The man skilled in the art has known for a long time a variety of means to perform a deletion, substitution or introduction in a nucleic acid sequence.

As will be understood by those of skill in the art, it can moreover be advantageous to modify a coding sequence to enhance its expression in a particular host. The genetic code is redundant with 64 possible codons, but most organisms typically use a subset of these codons. The codons that are utilized most often in a species are called optimal codons, and those not utilized very often are classified as rare or low-usage codons. Codons can be substituted to reflect the preferred codon usage of the host, in a process sometimes called “codon optimization” or “controlling for species codon bias.” Codon optimization for other host cells can be readily determined using codon usage tables or can be performed using commercially available software, such as CodonOp (www.idtdna.com/CodonOptfrom) from Integrated DNA Technologies. Optimized coding sequences containing codons preferred by a particular prokaryotic or eukaryotic host (Murray et al, 1989, Nucl Acids Res. 17: 477-508) can be prepared, for example, to increase the rate of translation or to produce recombinant RNA transcripts having desirable properties, such as a longer half-life, as compared with transcripts produced from a non-optimized sequence. Translation stop codons can also be modified to reflect host preference. For example, typical stop codon for monocotyledonous plants is UGA, whereas insects and E. coli commonly use UAA as the stop codon (Dalphin et al, 1996, Nucl Acids Res. 24: 216-8).

A “non-integrative” lentiviral vector means that, when this lentiviral vector is in a cell, it does not integrate into the host cell genome. A non-integrative lentiviral vector particle relates to a lentiviral vector particle that comprises a non-integrative lentiviral vector. It can also be termed integration-defective lentiviral vectors or non-integrating lentiviral vectors.

HPV-induced cancers are also known as cancers associated with HPV (Human Papillomavirus). Indeed, when HPV infections are not successfully controlled by the immune system of an infected host. When a high-risk HPV infection persists for many years, it can lead to cell changes that, if untreated, may get worse over time and become cancer.

### Lentiviral Vector According to the Invention

The inventors have conceived novel therapeutic and prophylactic lentiviral vectors based vaccines against HPV-induced cancers.

In particular, the present invention relates to a lentiviral vector comprising at least four distinct nucleic acid sequences selected from a group of particular non-oncogenic HPV antigens.

By distinct nucleic acid sequences, it is meant that the at least four nucleic acid sequences comprised in the lentiviral vector are all different, i.e. that each of them is a different member of the group of particular non-oncogenic HPV antigens.

The group of non-oncogenic HPV antigens is the following:
a nucleic acid sequence encoding a non-oncogenic Human papillomavirus 16 (HPV16) protein E6 antigen;
a nucleic acid sequence encoding a non-oncogenic Human papillomavirus (HPV16) protein E7 antigen;
a nucleic acid sequence encoding a non-oncogenic Human papillomavirus (HPV18) protein E6 antigen; and
a nucleic acid sequence encoding a non-oncogenic Human papillomavirus (HPV18) protein E7 antigen.

Unlike most HPV proteins that are lost after integration of HPV, E6 and E7 proteins are continuously expressed in HPV induced tumors (Ghittoni, Raffaella et al. Virus genes vol. 40, 1 (2010): 1-13; Morrow, Matthew P et al. Expert review of vaccines vol. 12, 3 (2013): 271-83). These proteins interfere with cell functions and are known to play an important role in HPV associated cancerogenesis. (Tomaid, Vjekoslav. Cancers vol. 8, 10 95. 19 Oct. 2016; Ghittoni, Raffaella et al. Virus genes vol. 40, 1 (2010): 1-13). E6 and E7 are thought to interfere with multiple pathways but most importantly, E6 protein expression in the cell leads to ubiquitin-mediated degradation of the tumor suppressor p53 by direct interaction with the cellular E3 ubiquitin ligase, E6AP (Huibregtse, J M et al. The EMBO journal vol. 10, 13 (1991): 4129-35; Martinez-Zapien, Denise et al. Nature vol. 529, 7587 (2016): 541-5) and E7 binds to Rb protein hereby disrupting the interaction between Rb and E2F and releasing E2F factor (Cassetti, M Cristina et al. Vaccine vol. 22, 3-4 (2004): 520-7).

As E6 and E7 protein are expressed in all HPV induced cancers, it was decided to include antigens from these proteins for the two major subtypes (HPV16 and HPV18) in lentiviral vectors of the invention. In order to develop a vaccine from E6 and E7 antigen, it is of major importance to abolish the oncogenic risk associated to these proteins.

Non-oncogenic E6 and E7 proteins are thus implemented in the invention. By “non-oncogenic E6 and E7 HPV proteins”, it means that their encoding sequences were modified to remove p53, Mi2b and Rb binding sites as well as PDZ binding motif. In a particular embodiment, as partial mutation of the binding sites of the E6 and E7 HPV proteins did not allow to fully abolish Rb binding, said sites were in particular fully removed from the sequences implemented in the present invention.

A lentiviral vector according to the invention may be single-stranded or double-stranded. A lentiviral vector according to the invention may be an RNA or DNA molecule.

In the context of the present invention, a “lentiviral vector” means a non-replicating vector for the transduction of a host cell with a transgene comprising cis-acting lentiviral RNA or DNA sequences, and requiring essential lentiviral proteins (e.g., Gag, Pol, and/or Env) and accessory proteins (e.g, Tat, Rev) that are provided in trans. The lentiviral vector lacks expression of all functional HIV proteins. The lentiviral vector genome may be present in the form of an RNA or DNA molecule, depending on the stage of production or development of said retroviral vectors.

In a preferred embodiment, a lentiviral vector of the invention is a non-integrative lentiviral vector.

Non-integrating lentiviral vectors have been designed to mitigate the risks of potential oncogenesis linked to insertional mutagenesis events, particularly for vaccination purposes. Examples of non-integrating lentiviral vectors are provided in Coutant et al., PLOS ONE 7(11):e48644 (2012), Karwacz et al., J. Virol. 83(7):3094-3103 (2009), Negri et al., Molecular Therapy 15(9):1716-1723 (2007); and Hu et al., Vaccine 28:6675-6683 (2010). Consequently, it has been reported that a non-integrating lentiviral vector system can mitigate the potential risk of insertional mutagenesis as compared to an integrating system (Hu et al., Vaccine 28:6675-6683 (2010)). It has been further reported that in some functional analysis, both the magnitude and quality of the immune responses elicited by DC-directed integration-defective lentiviral vectors (IDLVs) are comparable to that of its integrating counterpart. Thus, integration-defective lentiviral vectors (IDLVs) have been considered safer vectors than integrating vectors for human administration, with comparable effectiveness.

In addition, deletion in the U3 region of the 3′ LTR of the viral promoter and enhancer sequences in self-inactivating lentiviral vectors limits the likelihood of endogenous promoter activation. These concerns with safety directly address the experiences gained from the SCID-X1 gene therapy trial carried out in 1998-1999, performed with Moloney virus-based retroviral vectors on children suffering from a rare form of X-linked (SCID-X1 gene) severe immunodeficiency disease (Cavazzana-Calvo et al., 2000, Science., 288(5466):669-72). During this trial, four of nine children developed leukemia as a result of the integration of the Moloney-derived retroviral vector at close proximity to the human LM02 proto-oncogene (Hacein-Bey-Abina et al., 2008, J. Clin. Invest., 118(9):3132-3142). It was demonstrated that malignancy was the consequence of the proximity of the viral U3 promoter/enhancer to the LM02 proto-oncogene. As a result, safety is a major concern for the administration of lentivectors to humans.

Accordingly, a lentiviral vector according to the invention may comprise long terminal repeats (LTRs) sequences in cis as known in the art and in particular comprise a 3′ long terminal repeat (LTR) which is devoid of its U3 promoter sequence (Miyoshi H et al, 1998, J Virol. 72(1 0):81 50-7; Zufferey et al., 1998, J V/ro/72(12):9873-80).

Enhancers are cis-acting sequences, which can act as transcriptional activators at a distance. They have been widely employed in viral derived vectors because they appear to be the most efficient for obtaining transgene strong expression in a variety of cell types, in particular DCs (Chinnasamy et al., 2000, Hum Gene Ther 11(13):1901-9; Rouas et al., 2008, Cancer Gene Ther 9(9):715-24; Kimura et al., 2007, Mol Ther 15(7):1390-9; Gruh et al., 2008, J Gene Med 10(1) 21-32). However, given the safety issue of insertional mutagenesis, such transcriptional enhancer sequences should be deleted from the lentiviral vector constructs to abolish the risk of insertional mutagenesis by enhancer proximity effect. This enhancer proximity effect is by far the most frequent mechanism of insertional mutagenesis and is the only effect described in human or animal cases of tumorigenic events after gene transfer.

Accordingly, a lentiviral vector according to the invention may not comprise a constitutive enhancer sequence.

Previous studies have reported on the replacement of viral promoters by DC-specific promoters deriving from major histocompatibility complex class II genes (MHC class II) (Kimura et al., 2007, Mol Ther 15(7):1390-9) and dectin-2 genes (Lopes et al., 2008, J Virol 82(1):86-95). The dectin-2 gene promoter used in Lopes et al. contains a putative enhancer and an adenoviral conserved sequence (inverted terminal repeats in adenovirus promoter) (Bonkabara et al., 2001, J. Immunology, 167:6893-6900). The MHC class II gene promoter used by Kimura et al. does not contain any known enhancer.

Yet, without an enhancer, the MHC class II promoter was found not to provide sufficient transgene expression in DCs, when administered intravenously. In particular, lentiviral vectors including MHC class II promoters did not provoke an immune reaction in immunocompetent C57BL/6 mice, in contrast to the immune responses observed with CMV promoters/enhancers. Although integration and persistent transgene expression were observed after injection in mice, the lentiviral vectors transcribed through MHC class II promoters failed to stimulate an antigen-specific CD8+ cytotoxic T-lymphocyte response, even after vaccination boost. The authors of these studies therefore concluded that the use of MHC class II promoters was of interest only for applications where persistence of expression is sought as in gene replacement therapy, but not in the context of immunotherapy. Of note, MHC class II promoters are expressed poorly in most cell types.

Thus, the MHC class II promoter is not an adequate promoter for lentiviral vectors for induction of an immune response against an antigen via IV injection. Moreover, the dectin-2 promoter is expressed poorly in most cell types and appears to contain an enhancer. Thus, the dectin-2 promoter is not a good promoter for lentiviral vectors for safety reasons.

Accordingly, a lentiviral vector according to the invention may comprise an MHC Class I promoter, i.e. the nucleic acid sequences encoding antigens of a lentiviral vector according to the invention may be under the control of an MHC Class I promoter.

An appropriate MHC Class I promoter may be selected from the group consisting of a β2-microglobulin promoter, a HLA-A2 promoter, a HLA-B7 promoter, a HLA-Cw5 promoter, a HLA-E promoter or a HLA-F promoter and is more particularly a β2-microglobulin promoter.

MHC Class I promoters are dendritic-specific (APCs) in that expression of the promoter in BDCA+ dendritic cells is higher than the expression in kidney, smooth muscle, liver, and heart cells. They also have relatively high expression in other transduced cell types, for example, expression of the promoter in BDCA+ dendritic cells is only 12-100 times the expression of that promoter in skeletal muscle cells, in contrast to 900 times with the MHCII HLA-DRα promoter.

This promoter drives in particular the transcription of the nucleic acid sequences encoding HPV antigens in a lentiviral vector of the invention.

Said promoter can be a naturally occurring or a synthetic MHC Class I promoter, obtained using well known molecular biological techniques.

A lentiviral vector according to the invention may comprise a cPPT/CTS sequence, such as described in EP2169073. This cPPT/CTS sequence may in particular be the sequence set forth as sequence SEQ ID NO: 37.

Indeed, efficient integration and replication in non-dividing cells generally requires the presence of two cis-acting sequences at the center of the lentiviral genome, the central polypurine tract (cPPT) and the central termination sequence (CTS). This leads to the formation of a triple-stranded DNA structure called the central DNA “flap”, which acts as a signal for uncoating of the pre-integration complex at the nuclear pore and efficient importation of the expression cassette into the nucleus of non-dividing cells, such as dendritic cells.

A lentiviral vector of the invention may comprise a Woodchuck hepatitis B virus (WHV) Post-Transcriptional Regulatory Element (WPRE), which allows a more stable expression of the transgene in vivo, and in particular a mutant form of the woodchuck hepatitis B virus (WHV) post-transcriptional regulatory element (WPRE).

The mutated Woodchuck Posttranscriptional Regulatory Element (mWPRE) is characterized in that point mutations are introduced to avoid expression of the X protein contained in the WPRE region as said X protein may have oncogenic properties (Kingsman et al., Gene Ther. 2005 January; 12(1):3-4).

The mutant form of the woodchuck hepatitis B virus (WHV) post-transcriptional regulatory element (WPRE) comprised in a lentiviral vector of the invention may in particular have the nucleic acid sequence set forth as sequence SEQ ID NO: 38.

In a particular embodiment, a lentiviral vector according to the invention, and in particular a non-integrative lentiviral vector of the invention:
(i) comprises at least one nucleic acid sequence encoding a non-oncogenic Human papillomavirus (HPV16) protein E6 antigen, at least one nucleic acid sequence encoding a non-oncogenic Human papillomavirus (HPV16) protein E7 antigen, at least one nucleic acid sequence encoding a non-oncogenic Human papillomavirus (HPV18) protein E6 antigen, and at least one nucleic acid sequence encoding a non-oncogenic Human papillomavirus (HPV18) protein E7 antigen;
(ii) comprises a 3′ long terminal repeat (LTR) which is devoid of its U3 promoter sequence;
(iii) does not comprise a constitutive enhancer sequence;
(iv) comprises an MHC Class I promoter, and in particular a 02-microglobulin promoter;
(v) comprises a cPPT/CTS sequence, having in particular the sequence set forth as sequence SEQ ID NO: 37; and
(vi) comprises a mutant form of the woodchuck hepatitis B virus (WHV) post-transcriptional regulatory element (WPRE), having in particular the nucleic acid sequence set forth as sequence SEQ ID NO: 38.

As previously mentioned, a lentiviral vector according to the invention is characterized in that it comprises:
at least one nucleic acid sequence encoding a non-oncogenic Human papillomavirus (HPV16) protein E6 antigen,
at least one nucleic acid sequence encoding a non-oncogenic Human papillomavirus (HPV16) protein E7 antigen,
at least one nucleic acid sequence encoding a non-oncogenic Human papillomavirus (HPV18) protein E6 antigen, and
at least one nucleic acid sequence encoding a non-oncogenic Human papillomavirus (HPV18) protein E7 antigen.

The at least four, and in particular the four, distinct nucleic acid sequences encoding HPV antigens of a lentiviral vector of the invention may in particular be fused together, forming a single antigenic nucleic acid sequence encoding a single antigenic fusion protein under the control of a single promoter sequence, in particular (i) in the absence of any linking sequence (also termed spacer herein) between each of the at least four distinct nucleic acid sequences or (ii) with a linking sequence (or spacer) between at least two of the at least four distinct nucleic acid sequences, and more particularly with a linking sequence (or spacer) between each of the at least four distinct nucleic acid sequences.

A nucleic acid sequence encoding a non-oncogenic Human papillomavirus 16 (HPV16) protein E6 antigen may in particular have a nucleic acid sequence encoding an amino acid sequence having at least 80% sequence identity with the amino acid sequence set forth as SEQ ID NO: 7.

As described herein, an amino acid sequence having at least 80% amino acid identity with a reference amino acid sequence encompasses amino acid sequences having at least 81%, 82%, 83%, 84%, 85%, 86%, 87%, 88%, 89%, 90%, 91%, 92%, 93%, 94%, 95%, 96%, 97%, 98% and 99% amino acid identity with the said reference amino acid sequence.

In particular, a nucleic acid sequence encoding a non-oncogenic Human papillomavirus 16 (HPV16) protein E6 antigen may in particular have a nucleic acid sequence having at least 80% sequence identity with a nucleic acid sequence selected from the group consisting of the nucleic acid sequences set forth as SEQ ID NO: 2, SEQ ID NO: 3, SEQ ID NO: 4, SEQ ID NO: 5 and SEQ ID NO: 6.

As described herein, a nucleic acid sequence having at least 80% nucleotide identity with a reference nucleic acid sequence encompasses nucleic acid sequences having at least 81%, 82%, 83%, 84%, 85%, 86%, 87%, 88%, 89%, 90%, 91%, 92%, 93%, 94%, 95%, 96%, 97%, 98% and 99% nucleotide identity with the said reference nucleic acid sequence.

In a particular embodiment, a nucleic acid sequence encoding a non-oncogenic Human papillomavirus 16 (HPV16) protein E6 antigen may in particular have a nucleic acid sequence selected from the group consisting of the nucleic acid sequences set forth as SEQ ID NO: 2, SEQ ID NO: 3, SEQ ID NO: 4, SEQ ID NO: 5 and SEQ ID NO: 6.

A nucleic acid sequence encoding a non-oncogenic Human papillomavirus 16 (HPV16) protein E6 antigen may in particular have a nucleic acid sequence encoding an amino acid sequence having at least 80% sequence identity with an amino acid sequence selected from the group consisting of the amino acid sequences set forth as SEQ ID NO: 8, SEQ ID NO: 9, SEQ ID NO: 10, SEQ ID NO: 11 and SEQ ID NO: 12.

In a particular embodiment, a nucleic acid sequence encoding a non-oncogenic Human papillomavirus 16 (HPV16) protein E6 antigen may in particular have a nucleic acid sequence encoding an amino acid sequence selected from the group consisting of the amino acid sequences set forth as SEQ ID NO: 8, SEQ ID NO: 9, SEQ ID NO: 10, SEQ ID NO: 11 and SEQ ID NO: 12.

A nucleic acid sequence encoding a non-oncogenic Human papillomavirus 16 (HPV16) protein E7 antigen may in particular have a nucleic acid sequence encoding an amino acid sequence having at least 68% sequence identity with the amino acid sequence set forth as SEQ ID NO: 16.

As described herein, an amino acid sequence having at least 68% amino acid identity with a reference amino acid sequence encompasses amino acid sequences having at least 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% and 99% amino acid identity with the said reference amino acid sequence.

In particular, a nucleic acid sequence encoding a non-oncogenic Human papillomavirus 16 (HPV16) protein E7 antigen may in particular have a nucleic acid sequence having at least 80% sequence identity with a nucleic acid sequence selected from the group consisting of the nucleic acid sequences set forth as SEQ ID NO: 14 and SEQ ID NO: 15.

As described herein, a nucleic acid sequence having at least 80% nucleotide identity with a reference nucleic acid sequence encompasses nucleic acid sequences having at least 81%, 82%, 83%, 84%, 85%, 86%, 87%, 88%, 89%, 90%, 91%, 92%, 93%, 94%, 95%, 96%, 97%, 98% and 99% nucleotide identity with the said reference nucleic acid sequence.

In a particular embodiment, a nucleic acid sequence encoding a non-oncogenic Human papillomavirus 16 (HPV16) protein E7 antigen may in particular have a nucleic acid sequence selected from the group consisting of the nucleic acid sequences set forth as SEQ ID NO: 14 and SEQ ID NO: 15.

A nucleic acid sequence encoding a non-oncogenic Human papillomavirus 16 (HPV16) protein E7 antigen may in particular have a nucleic acid sequence encoding an amino acid sequence having at least 80% sequence identity with an amino acid sequence selected from the group consisting of the amino acid sequences set forth as SEQ ID NO: 17 and SEQ ID NO: 18.

In a particular embodiment, a nucleic acid sequence encoding a non-oncogenic Human papillomavirus 16 (HPV16) protein E7 antigen may in particular have a nucleic acid sequence encoding an amino acid sequence selected from the group consisting of the amino acid sequences set forth as SEQ ID NO: 17 and SEQ ID NO: 18.

A nucleic acid sequence encoding a non-oncogenic Human papillomavirus 18 (HPV18) protein E6 antigen may in particular have a nucleic acid sequence encoding an amino acid sequence having at least 60% sequence identity with the amino acid sequence set forth as SEQ ID NO: 24.

As described herein, an amino acid sequence having at least 60% amino acid identity with a reference amino acid sequence encompasses amino acid sequences having at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% and 99% amino acid identity with the said reference amino acid sequence.

In particular, a nucleic acid sequence encoding a non-oncogenic Human papillomavirus 18 (HPV18) protein E6 antigen may in particular have a nucleic acid sequence having at least 80% sequence identity with a nucleic acid sequence selected from the group consisting of the nucleic acid sequences set forth as SEQ ID NO: 20, SEQ ID NO: 21, SEQ ID NO: 22 and SEQ ID NO: 23.

As described herein, a nucleic acid sequence having at least 80% nucleotide identity with a reference nucleic acid sequence encompasses nucleic acid sequences having at least 81%, 82%, 83%, 84%, 85%, 86%, 87%, 88%, 89%, 90%, 91%, 92%, 93%, 94%, 95%, 96%, 97%, 98% and 99% nucleotide identity with the said reference nucleic acid sequence.

In a particular embodiment, a nucleic acid sequence encoding a non-oncogenic Human papillomavirus 18 (HPV18) protein E6 antigen may in particular have a nucleic acid sequence selected from the group consisting of the nucleic acid sequences set forth as SEQ ID NO: 20, SEQ ID NO: 21, SEQ ID NO: 22 and SEQ ID NO: 23.

A nucleic acid sequence encoding a non-oncogenic Human papillomavirus 18 (HPV18) protein E6 antigen may in particular have a nucleic acid sequence encoding an amino acid sequence having at least 80% sequence identity with an amino acid sequence selected from the group consisting of the amino acid sequences set forth as SEQ ID NO: 25, SEQ ID NO: 26, SEQ ID NO: 27 and SEQ ID NO: 28.

In a particular embodiment, a nucleic acid sequence encoding a non-oncogenic Human papillomavirus 18 (HPV18) protein E6 antigen may in particular have a nucleic acid sequence encoding an amino acid sequence selected from the group consisting of the amino acid sequences set forth as SEQ ID NO: 25, SEQ ID NO: 26, SEQ ID NO: 27 and SEQ ID NO: 28.

A nucleic acid sequence encoding a non-oncogenic Human papillomavirus 18 (HPV18) protein E7 antigen may in particular have a nucleic acid sequence encoding an amino acid sequence having at least 83% sequence identity with the amino acid sequence set forth as SEQ ID NO: 33.

As described herein, an amino acid sequence having at least 83% amino acid identity with a reference amino acid sequence encompasses amino acid sequences having at least 84%, 85%, 86%, 87%, 88%, 89%, 90%, 91%, 92%, 93%, 94%, 95%, 96%, 97%, 98% and 99% amino acid identity with the said reference amino acid sequence.

In particular, a nucleic acid sequence encoding a non-oncogenic Human papillomavirus 18 (HPV18) protein E7 antigen may in particular have a nucleic acid sequence having at least 80% sequence identity with a nucleic acid sequence selected from the group consisting of the nucleic acid sequences set forth as SEQ ID NO: 30, SEQ ID NO: 31 and SEQ ID NO: 32.

As described herein, a nucleic acid sequence having at least 80% nucleotide identity with a reference nucleic acid sequence encompasses nucleic acid sequences having at least 81%, 82%, 83%, 84%, 85%, 86%, 87%, 88%, 89%, 90%, 91%, 92%, 93%, 94%, 95%, 96%, 97%, 98% and 99% nucleotide identity with the said reference nucleic acid sequence.

In a particular embodiment, a nucleic acid sequence encoding a non-oncogenic Human papillomavirus 18 (HPV18) protein E7 antigen may in particular have a nucleic acid sequence selected from the group consisting of the nucleic acid sequences set forth as SEQ ID NO: 30, SEQ ID NO: 31 and SEQ ID NO: 32.

A nucleic acid sequence encoding a non-oncogenic Human papillomavirus 18 (HPV18) protein E7 antigen may in particular have a nucleic acid sequence encoding an amino acid sequence having at least 80% sequence identity with an amino acid sequence selected from the group consisting of the amino acid sequences set forth as SEQ ID NO: 34, SEQ ID NO: 35 and SEQ ID NO: 36.

In a particular embodiment, a nucleic acid sequence encoding a non-oncogenic Human papillomavirus 18 (HPV18) protein E7 antigen may in particular have a nucleic acid sequence encoding an amino acid sequence selected from the group consisting of the amino acid sequences set forth as SEQ ID NO: 34, SEQ ID NO: 35 and SEQ ID NO: 36.

As indicated above, a lentiviral vector according to the invention comprises:
at least one nucleic acid sequence encoding a non-oncogenic Human papillomavirus (HPV16) protein E6 antigen (also termed herein noE6-HPV16),
at least one nucleic acid sequence encoding a non-oncogenic Human papillomavirus (HPV16) protein E7 antigen (also termed herein noE7-HPV16),
at least one nucleic acid sequence encoding a non-oncogenic Human papillomavirus (HPV18) protein E6 antigen (also termed herein noE6-HPV18), and
at least one nucleic acid sequence encoding a non-oncogenic Human papillomavirus (HPV18) protein E7 antigen (also termed herein noE7-HPV18).

In a particular embodiment, a lentiviral vector according to the invention, and in particular a non-integrative lentiviral vector of the invention:
(i) comprises a nucleic acid sequence encoding a non-oncogenic Human papillomavirus 16 (HPV16) protein E6 antigen; a nucleic acid sequence encoding a non-oncogenic Human papillomavirus (HPV16) protein E7 antigen; a nucleic acid sequence encoding a non-oncogenic Human papillomavirus (HPV18) protein E6 antigen and a nucleic acid sequence encoding a non-oncogenic Human papillomavirus (HPV18) protein E7 antigen;
the nucleic acid sequences encoding non-oncogenic HPV antigens being in particular fused together, forming a single antigenic nucleic acid sequence encoding a single antigenic fusion protein; under the control of a single promoter sequence, more particularly in the absence of any linking sequence between each of them;
(ii) comprises a 3′ long terminal repeat (LTR) which is devoid of its U3 promoter sequence;
(iii) does not comprise a constitutive enhancer sequence;
(iv) comprises an MHC Class I promoter, and in particular a 02-microglobulin promoter;
(v) comprises a cPPT/CTS sequence, having in particular the sequence set forth as sequence SEQ ID NO: 37; and
(vi) comprises a mutant form of the woodchuck hepatitis B virus (WHV) post-transcriptional regulatory element (WPRE), having in particular the nucleic acid sequence set forth as sequence SEQ ID NO: 38.

A lentiviral vector according to the invention may more particularly comprise:
a nucleic acid sequence encoding a non-oncogenic Human papillomavirus (HPV16) protein E6 antigen, the nucleic acid sequence having at least 80% sequence identity with a nucleic acid sequence selected from the group consisting of the nucleic acid sequences set forth as SEQ ID NO: 2, SEQ ID NO: 3, SEQ ID NO: 4, SEQ ID NO: 5 and SEQ ID NO: 6, the nucleic acid sequence being in particular selected from the group consisting of the nucleic acid sequences set forth as SEQ ID NO: 2, SEQ ID NO: 3, SEQ ID NO: 4, SEQ ID NO: 5 and SEQ ID NO: 6;
at least one nucleic acid sequence encoding a non-oncogenic Human papillomavirus (HPV16) protein E7 antigen, the nucleic acid sequence having at least 80% sequence identity with a nucleic acid sequence selected from the group consisting of the nucleic acid sequences set forth as SEQ ID NO: 14 and SEQ ID NO: 15, the nucleic acid sequence being in particular selected from the group consisting of the nucleic acid sequences set forth as SEQ ID NO: 14 and SEQ ID NO: 15;
at least one nucleic acid sequence encoding a non-oncogenic Human papillomavirus (HPV18) protein E6 antigen, the nucleic acid sequence having at least 80% sequence identity with a nucleic acid sequence selected from the group consisting of the nucleic acid sequences set forth as SEQ ID NO: 20, SEQ ID NO: 21, SEQ ID NO: 22 and SEQ ID NO: 23, the nucleic acid sequence being in particular selected from the group consisting of the nucleic acid sequences set forth as SEQ ID NO: 20, SEQ ID NO: 21, SEQ ID NO: 22 and SEQ ID NO: 23; and
at least one nucleic acid sequence encoding a non-oncogenic Human papillomavirus (HPV18) protein E7 antigen, the nucleic acid sequence having at least 80% sequence identity with a nucleic acid sequence selected from the group consisting of the nucleic acid sequences set forth as SEQ ID NO: 30, SEQ ID NO: 31 and SEQ ID NO: 32, the nucleic acid sequence being in particular selected from the group consisting of the nucleic acid sequences set forth as SEQ ID NO: 30, SEQ ID NO: 31 and SEQ ID NO: 32;
the nucleic acid sequences encoding non-oncogenic HPV antigens being in particular fused together, forming a single antigenic nucleic acid sequence encoding a single antigenic fusion protein; under the control of a single promoter sequence, more particularly in the absence of any linking sequence between each of them.

The at least four, and in particular the four, distinct nucleic acid sequences encoding HPV antigens in a lentiviral vector according to the invention, and in particular a non-integrative lentiviral vector of the invention, may be in any order in the traditional 5′ to 3′ reading direction (from 5′ end to 3′ end).

In particular, the four distinct nucleic acid sequences encoding HPV antigens noE6-HPV16, noE7-HPV16, noE6-HPV18 and noE7-HPV18 as defined above may be in any order, in the traditional 5′ end to 3′ end reading direction, among the 24 possible combinations in lentiviral vectors according to the invention, and in particular non-integrative lentiviral vectors according to the invention.

In a particular embodiment, the order of the at least four distinct nucleic acid sequences, from 5′ end to 3′ end, is selected from the group consisting of:
(a) nucleic acid sequence encoding a non-oncogenic Human papillomavirus (HPV16) protein E7 antigen—nucleic acid sequence encoding a non-oncogenic Human papillomavirus (HPV16) protein E6 antigen—nucleic acid sequence encoding a non-oncogenic Human papillomavirus (HPV18) protein E7 antigen—nucleic acid sequence encoding a non-oncogenic Human papillomavirus (HPV18) protein E6 antigen;
(b) nucleic acid sequence encoding a non-oncogenic Human papillomavirus (HPV16) protein E6 antigen—nucleic acid sequence encoding a non-oncogenic Human papillomavirus (HPV18) protein E6 antigen—nucleic acid sequence encoding a non-oncogenic Human papillomavirus (HPV16) protein E7 antigen—nucleic acid sequence encoding a non-oncogenic Human papillomavirus (HPV18) protein E7 antigen;
(c) nucleic acid sequence encoding a non-oncogenic Human papillomavirus (HPV18) protein E6 antigen—nucleic acid sequence encoding a non-oncogenic Human papillomavirus (HPV16) protein E6 antigen—nucleic acid sequence encoding a non-oncogenic Human papillomavirus (HPV18) protein E7 antigen—nucleic acid sequence encoding a non-oncogenic Human papillomavirus (HPV16) protein E7 antigen; and
(d) nucleic acid sequence encoding a non-oncogenic Human papillomavirus (HPV18) protein E7 antigen—nucleic acid sequence encoding a non-oncogenic Human papillomavirus (HPV18) protein E6 antigen—nucleic acid sequence encoding a non-oncogenic Human papillomavirus (HPV16) protein E7 antigen—nucleic acid sequence encoding a non-oncogenic Human papillomavirus (HPV16) protein E6 antigen,
the nucleic acid sequences encoding non-oncogenic HPV antigens being in particular fused together, forming a single antigenic nucleic acid sequence encoding a single antigenic fusion protein; under the control of a single promoter sequence, more particularly in the absence of any linking sequence between each of them.

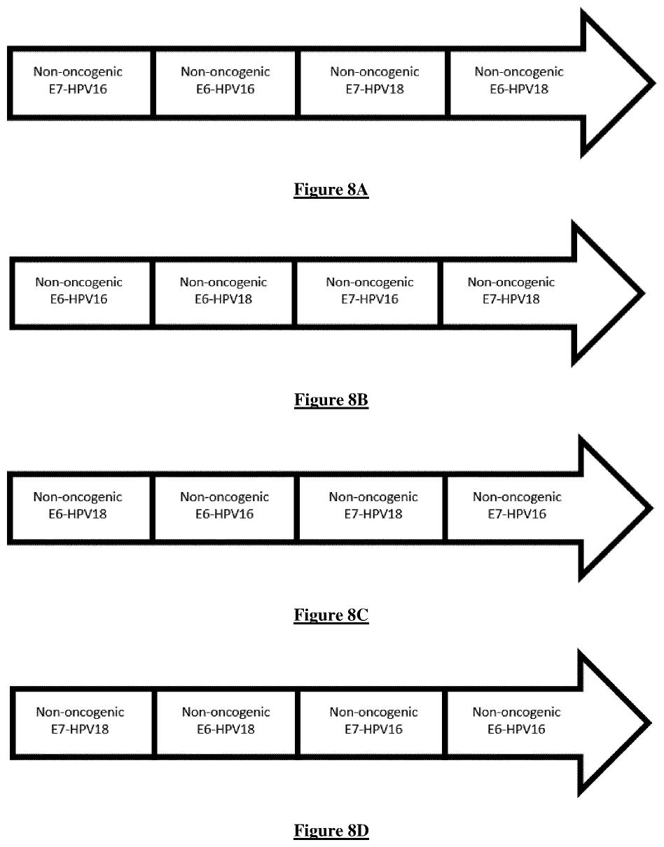

These orders are represented in FIGS. 8A to 8D.

The order of the at least four distinct nucleic acid sequences, from 5′ end to 3′ end, in a lentiviral vector according to the invention may more particularly be:
(d) nucleic acid sequence encoding a non-oncogenic Human papillomavirus (HPV18) protein E7 antigen—nucleic acid sequence encoding a non-oncogenic Human papillomavirus (HPV18) protein E6 antigen—nucleic acid sequence encoding a non-oncogenic Human papillomavirus (HPV16) protein E7 antigen—nucleic acid sequence encoding a non-oncogenic Human papillomavirus (HPV16) protein E6 antigen,
the nucleic acid sequences encoding non-oncogenic HPV antigens being in particular fused together, forming a single antigenic nucleic acid sequence encoding a single antigenic fusion protein; under the control of a single promoter sequence, more particularly in the absence of any linking sequence between each of them.

Preferably, the order of the at least four distinct nucleic acid sequences, from 5′ end to 3′ end, in a lentiviral vector according to the invention is:
(a) nucleic acid sequence encoding a non-oncogenic Human papillomavirus (HPV16) protein E7 antigen—nucleic acid sequence encoding a non-oncogenic Human papillomavirus (HPV16) protein E6 antigen—nucleic acid sequence encoding a non-oncogenic Human papillomavirus (HPV18) protein E7 antigen—nucleic acid sequence encoding a non-oncogenic Human papillomavirus (HPV18) protein E6 antigen,
the nucleic acid sequences encoding non-oncogenic HPV antigens being in particular fused together, forming a single antigenic nucleic acid sequence encoding a single antigenic fusion protein; under the control of a single promoter sequence, more particularly in the absence of any linking sequence between each of them.

More preferably, the order of the at least four distinct nucleic acid sequences, from 5′ end to 3′ end, in a lentiviral vector according to the invention is:
(a) nucleic acid sequence encoding a non-oncogenic Human papillomavirus (HPV16) protein E7 antigen—nucleic acid sequence encoding a non-oncogenic Human papillomavirus (HPV16) protein E6 antigen—nucleic acid sequence encoding a non-oncogenic Human papillomavirus (HPV18) protein E7 antigen—nucleic acid sequence encoding a non-oncogenic Human papillomavirus (HPV18) protein E6 antigen,
the nucleic acid sequences encoding non-oncogenic HPV antigens being fused together and forming a single antigenic nucleic acid sequence encoding a single antigenic fusion protein; under the control of a single promoter sequence, in the absence of any linking sequence between each of them.

These four different groups of antigens constructs have been respectively implemented in the present examples in:
the lentiviral vector filed at the Collection Nationale de Cultures de Microorganismes (CNCM) on Oct. 21, 2021 under accession number I-5759 (above order (a));
the lentiviral vector filed at the Collection Nationale de Cultures de Microorganismes (CNCM) on Oct. 21, 2021 under accession number I-5760 (above order (b));
the lentiviral vector filed at the Collection Nationale de Cultures de Microorganismes (CNCM) on Oct. 21, 2021 under accession number I-5761 (above order (c)); or
the lentiviral vector filed at the Collection Nationale de Cultures de Microorganismes (CNCM) on Oct. 21, 2021 under accession number I-5762 (above order (d)).

Accordingly, the antigen construct of the lentiviral vector filed at the CNCM under accession number I-5759 has the following nucleotidic sequence set forth as SEQ ID NO: 41:

(SEQ ID NO: 41)
atgcccggagacacccccaccctgcacgaatacatgctggacctgcagc
ccgaaaccaccgaccccgaccgcgctcactacaacatcgttacattctg
ttgtaaatgcgactccaccctgagaagatgcgtgcagtccacccacgtg
gacatcaggaccctggaggacctcctcatgggaaccctgggtatcgtct
gccccatcgcctcccaggcttttcaggacccccaggaaaggcccaggaa
gttgccccagctctgcaccgaactgcagaccaccattcatgacatcatc
ctcgaatgcgtgtactgcaagcagcagctcctgaggagggaggtgtacg
atttcgccttcagagacggctgtatcgtctacaggaacccctatgccgt
ctgcgacaaatgcctgaagttttattccaagatctccgagtacaggcac
tattgctacagcctgtatgggaccaccctggagcagcagtacaacaagc
ccctgtgcgacctcctgatcaggtgcatcaactgccagaagcccctgag
gttccacaacatccgcggcaggtggaccggaaggtgcatgtcctgctgc
aggtccgccggccccggacctaaagccaccctccaggacatcgttctcc
acctggagccccagaacgagatccccgtggactcagaagaggagaacga
cgagatcgacggcgtcaaccaccagcacctgcccgctcgcagagccgaa
ccccagagacacaccatgctctgcatgtgctgcaaatgcgaagcccgga
ttaagttggtggtggaaagcagcgccgacgatctgagggccttccagca
gctcttcctcaacaccctgtccttcgtgtgcccctggggggcgagcccg
gtagaaccatcccctacaagctgcccgatctgtgcacagagctgaacac
ctccctgcaggacatcgagatcacctgcgtctactgcaagaccgtgctg
gaactgaccgaggtgttcgaattcgccttcaaggacggcttcgtggtgt
acagggacagcattccccacgccgcctgccataagctggagaaactgac
caacaccggactgtataacctgctgatcaggtgtctgaggtgccagaag
gcagagaaactgagacatctgaacgagaaaaggaggttccacaatattg
ccgggcactgataa
and encodes the following amino acid sequence
set forth as SEQ ID NO: 42:
(SEQ ID NO: 42)
MPGDTPTLHEYMLDLQPETTDPDRAHYNIVTFCCKCDSTLRRCVQSTHV
DIRTLEDLLMGTLGIVCPIASQAFQDPQERPRKLPQLCTELQTTIHDII
LECVYCKQQLLRREVYDFAFRDGCIVYRNPYAVCDKCLKFYSKISEYRH
YCYSLYGTTLEQQYNKPLCDLLIRCINCQKPLRFHNIRGRWTGRCMSCC
RSAGPGPKATLQDIVLHLEPQNEIPVDSEEENDEIDGVNHQHLPARRAE
PQRHTMLCMCCKCEARIKLVVESSADDLRAFQQLFLNTLSFVCPWVGEP
GRTIPYKLPDLCTELNTSLQDIEITCVYCKTVLELTEVFEFAFKDGFVV
YRDSIPHAACHKLEKLTNTGLYNLLIRCLRCQKAEKLRHLNEKRRFHNI
AGH

The antigen construct of the lentiviral vector filed at the CNCM under accession number I-5760 has the following nucleotidic sequence set forth as SEQ ID NO: 43:

(SEQ ID NO: 43)
atgttccaggacccccaggagaggccccggaagttgccccagctgtgca
ccgagctgcagaccaccatccacgacatcatcctcgaatgcgtgtactg
caagcagcagctgctgaggagggaggtgtatgactttgccttcagagac
ggatgcattgtctacaggaacccctacgccgtgtgcgacaaatgcctga
agttctactccaagatcagcgagtacaggcactactgctactccctgta
cggcaccaccctcgaacagcagtacaacaaacccctgtgcgacctcctg
attaggtgcatcaactgccagaagcccctcaggttccacaacatccgcg
gccgctggaccggccgatgcatgtcttgctgcaggggccccgacgaccc
ctacaagctccccgacctgtgcaccgaactcaacacctccctgcaggac
atcgagatcacctgcgtgtattgcaagaccgtgctggagctgaccgagg
ttttcgaatttgcctttaaggacggcttcgtcgtgtatagggactccat
cccccacgccgcctgccataagctggagaagctcaccaacaccggactg
tataatctgctgatcaggtgcctcaggtgccagaaggcagaaaagctga
ggcatctcaacgagaagcgccggttccacaatattgccggccccggaga
cacccccacactccatgagtacatgctcgacctgcagcccgaaaccacc
gaccccgacagagcccactacaacatcgtgaccttctgctgcaagtgcg
actccaccctgagaagatgcgtgcagtccacccacgtggacatccgcac
actcgaagacctgctgatgggaaccctgggcatcgtgtgccccatcggc
cccgatgacaaggccaccttgcaggacatcgtgctgcacctggaaccac
agaacgagatccccgtcgactccgaagaagaaaacgacgaaatcgacgg
agtgaatcaccagcacctgcccgccagaagggccgagcctcagagacac
accatgctctgcatgtgctgcaaatgcgaagccaggattaagctggtgg
tggagagcagcgccgacgacctgagggccttccagcagctcttcctgaa
cacactgtccttcgtgtgcccctgggcctgataa

and encodes the following amino acid sequence set forth as SEQ ID NO: 44:

(SEQ ID NO: 44)
MFQDPQERPRKLPQLCTELQTTIHDIILECVYCKQQLLRREVYDFAFRD
GCIVYRNPYAVCDKCLKFYSKISEYRHYCYSLYGTTLEQQYNKPLCDLL
IRCINCQKPLRFHNIRGRWTGRCMSCCRGPDDPYKLPDLCTELNTSLQD
IEITCVYCKTVLELTEVFEFAFKDGFVVYRDSIPHAACHKLEKLTNTGL
YNLLIRCLRCQKAEKLRHLNEKRRFHNIAGPGDTPTLHEYMLDLQPETT
DPDRAHYNIVTFCCKCDSTLRRCVQSTHVDIRTLEDLLMGTLGIVCPIG
PDDKATLQDIVLHLEPQNEIPVDSEEENDEIDGVNHQHLPARRAEPQRH
TMLCMCCKCEARIKLVVESSADDLRAFQQLFLNTLSFVCPWA

The antigen construct of the lentiviral vector filed at the CNCM under accession number I-5761 has the following nucleotidic sequence set forth as SEQ ID NO: 45:

(SEQ ID NO: 45)
atgaggcggccctacaagctgcccgacctgtgcaccgagctgaacacct
ccctgcaggacatcgagatcacctgcgtgtactgcaagaccgtgctgga
gctgaccgaggtgttcgaattcgcattcaaggacggattcgtcgtgtat
agggacagcattccacacgccgcctgccacaagctggagaaattgacta
acaccggactgtataatctgctgatccggtgcctgaggtgtcagaaggc
cgagaagctgaggcatctgaacgagaaaaggagattccacaatatcgcc
ggacacttccaggacccccaggagaggcccaggaaactgccccagttgt
gcaccgagctccagacaaccatccacgacatcatcctggagtgcgtgta
ctgtaagcagcagttgctgaggagagaggtgtatgacttcgccttcaga
gacggatgcattgtctataggaacccctacgccgtgtgcgacaagtgcc
tgaagttctactccaagatcagtgagtacaggcattactgctacagcct
gtatggaaccacactggaacagcagtacaacaagcccctgtgcgacctc
ctgattaggtgcatcaactgccagaagcccctcaggttccacaacatcc
ggggcaggtggaccggaaggtgcatgtcctgctgcaggtccgccggccc
cggacctaaagccaccctccaggacatcgtgctgcacctggagccccag
aacgagatccccgtcgactcagaggaggagaacgacgaaattgacggcg
tcaaccaccagcacctgcccgctcgcagagccgaaccccagagacacac
catgctctgcatgtgctgcaaatgcgaggcccggattaagctggtggtg
gagagctccgccgacgatctgagagccttccagcagctcttcctgaaca
ccctgtccttcgtgtgcccctgggccggtcccggtgacacacctaccct
gcacgagtacatgctcgatctgcagcccgagaccaccgaccccgatcgc
gcacactacaacatcgtgaccttctgctgcaaatgtgacagcaccctga
gacggtgcgtccagtccacccacgttgacatccgcaccctcgaagacct
gctcatgggaaccctgggcatcgtgtgccccatcgcctgataa

and encodes the following amino acid sequence set forth as SEQ ID NO: 46:

(SEQ ID NO: 46)
MRRPYKLPDLCTELNTSLQDIEITCVYCKTVLELTEVFEFAFKDGFVVY
RDSIPHAACHKLEKLTNTGLYNLLIRCLRCQKAEKLRHLNEKRRFHNIA
GHFQDPQERPRKLPQLCTELQTTIHDIILECVYCKQQLLRREVYDFAFR
DGCIVYRNPYAVCDKCLKFYSKISEYRHYCYSLYGTTLEQQYNKPLCDL
LIRCINCQKPLRFHNIRGRWTGRCMSCCRSAGPGPKATLQDIVLHLEPQ
NEIPVDSEEENDEIDGVNHQHLPARRAEPQRHTMLCMCCKCEARIKLVV
ESSADDLRAFQQLFLNTLSFVCPWAGPGDTPTLHEYMLDLQPETTDPDR
AHYNIVTFCCKCDSTLRRCVQSTHVDIRTLEDLLMGTLGIVCPIA

The antigen construct of the lentiviral vector filed at the CNCM under accession number I-5762 has the following nucleotidic sequence set forth as SEQ ID NO: 47:

(SEQ ID NO: 47)
atgggccctaaggccaccctgcaggacatcgtgctgcacttggagcccc
agaacgagatccccgtggacagcgaggaggagaacgacgaaatcgacgg
cgtgaaccaccagcacctgcccgcaagaagggccgaaccccagaggcac
accatgctctgcatgtgctgcaaatgcgaggccaggatcaagctggtgg
tggaaagcagcgccgacgatctgagggcattccagcagctgttcctgaa
caccctctccttcgtgtgccctggggaacccggcaggaccatcccctat
aaactgcccgacctctgcaccgagctgaacacctccctgcaggacattg
agatcacctgcgtctactgcaaaaccgtcctggaactgaccgaggtgtt
cgagttcgccttcaaagacggcttcgtcgtgtacagggacagcatcccc
cacgccgcctgccataagctggagaaactgaccaacaccggcctgtaca
acctgctgatccggtgcctgagatgtcagaaggccgagaaactgaggca
cctcaacgagaaaaggagattccacaatattgccgggcccggcgacacc
ccaaccctgcacgaatacatgctcgacctgcagcccgaaaccaccgacc
ccgacagagcccactacaacatcgtgaccttctgctgcaagtgcgactc
caccctgagaagatgcgtgcagtccacccacgtggacatccgcacactc
gaagacctgctgatgggaaccctgggcatcgtgtgccccatcgcttccc
aggcctttcaggacccccaggaacggccaagaaagctgccccagctctg
caccgaactgcagaccaccatccacgacatcatcctggaatgcgtctac
tgtaagcagcagttgctgaggagggaggtgtatgatttcgccttcagag
acggctgcatcgtctacaggaacccctacgccgtgtgcgacaaatgcct
gaagttctactccaagatctccgaatacagacactattgctacagcctg
tacggcaccaccctcgaacagcagtacaacaaacccctgtgcgacctcc
tgatcaggtgcatcaactgccagaagcccctccggttccacaacatccg
aggaagatggaccggccggtgcatgtcctgctgcaggtcctgataa

and encodes the following amino acid sequence set forth as SEQ ID NO: 48:

(SEQ ID NO: 48)
MGPKATLQDIVLHLEPQNEIPVDSEEENDEIDGVNHQHLPARRAEPQRH
TMLCMCCKCEARIKLVVESSADDLRAFQQLFLNTLSFVCPGEPGRTIPY
KLPDLCTELNTSLQDIEITCVYCKTVLELTEVFEFAFKDGFVVYRDSIP
HAACHKLEKLTNTGLYNLLIRCLRCQKAEKLRHLNEKRRFHNIAGPGDT
PTLHEYMLDLQPETTDPDRAHYNIVTFCCKCDSTLRRCVQSTHVDIRTL
EDLLMGTLGIVCPIASQAFQDPQERPRKLPQLCTELQTTIHDIILECVY
CKQQLLRREVYDFAFRDGCIVYRNPYAVCDKCLKFYSKISEYRHYCYSL
YGTTLEQQYNKPLCDLLIRCINCQKPLRFHNIRGRWTGRCMSCCRS

A lentiviral vector according to the invention may in particular comprise a nucleic acid sequence which encodes an amino acid sequence having at least 90% sequence identity with the amino acid sequence set forth as SEQ ID NO: 42, the nucleic acid sequence being in particular the nucleic acid sequence SEQ ID NO: 41.

As described herein, a amino acid sequence having at least 90% identity with a reference amino acid sequence encompasses amino acid sequences having at least 91%, 92%, 93%, 94%, 95%, 96%, 97%, 98%, 99% and 100% identity with the said reference amino acid sequence.

Accordingly, a lentiviral vector according to the invention may more particularly be selected from the group consisting of lentiviral vectors filed at the CNCM under accession numbers I-5759, I-5760, I-5761 and I-5762, and is in particular lentiviral vector filed at the CNCM under accession number I-5762. A lentiviral vector according to the invention may preferably be the lentiviral vector filed at the CNCM under accession number I-5759 and accordingly preferably comprises the nucleic acid sequence SEQ ID NO: 41.

### Lentiviral Vector Particles According to the Invention

Another object of the present invention relates to a lentiviral vector particle comprising at least one lentiviral vector according to the invention, and in particular at least one lentiviral vector as defined above.

A lentiviral vector particle according to the invention, which contains a lentiviral vector according to the invention, can be produced by recombinant technology known in the art upon transient transfection of cells, for example HEK 293T human cultured cells, by different DNA plasmids:
(i) a packaging plasmid, which expresses at least the Gag, Pol, Rev, Tat and, in some cases, structural and enzymatic proteins necessary for the packaging of the transfer construct;
(ii) a lentiviral vector according to the invention, containing an expression cassette (antigens) and HIV cis-acting factors necessary for packaging, reverse transcription, and integration; and
(iii) an envelope-encoding plasmid, in most cases the glycoprotein of vesicular stomatitis virus (VSV.G), a protein that allows the formation of mixed particles (pseudotypes) that can target a wide variety of cells, especially major histocompatibility (MHC) antigen-presenting cells (APCs), including DCs.

Such a method allows producing a recombinant vector particle according to the invention, comprising the following steps of:
i) transfecting a suitable host cell with a lentiviral vector according to the invention;
ii) transfecting said host cell with a packaging plasmid vector, containing viral DNA sequences encoding at least structural and polymerase activities of a retrovirus (preferably lentivirus); Such packaging plasmids are for example described in the art (Dull et al., 1998, J Virol, 72(11):8463-71; Zufferey et al., 1998, J Virol 72(12):9873-80).
iii) culturing said transfected host cell in order to obtain expression and packaging of said lentiviral vector into lentiviral vector particles; and
iv) harvesting the lentiviral vector particles resulting from the expression and packaging of step iii) in said cultured host cells.

In order to pseudotype the retroviral particles of the invention, the host cell can be further transfected with one or several envelope DNA plasmid(s) encoding viral envelope protein(s), preferably a VSV-G envelope protein.

This procedure allows obtaining transient production of lentiviral particle vectors by the transfected cells. However, the lentiviral particle vectors may also be continuously produced by cells by stably inserting the packaging genes, the proviral coding DNA, and the envelope gene into the cellular genome. This allows the continuous production of lentiviral particle vectors by the cells without the need for transient transfection. Of course, a combination of these procedures can be used, with some of the DNAs/plasmids integrated into the cellular genome and others provided by transient transfection.

A lentiviral vector particle may be a non-integrating lentiviral vector particle. Non-integrating vector particles have one or more mutations that eliminate most or all of the integrating capacity of the lentiviral vector particles. For, example, a non-integrating vector particle can contain mutation(s) in the integrase encoded by the lentiviral pol gene that cause a reduction in integrating capacity.

A lentiviral vector particle according to the invention in particular comprises a non-integrating lentiviral vector of the invention.

A lentiviral vector particle according to the invention may comprise a vesicular stomatitis virus glycoprotein (VSVG), in particular a VSV-G Indiana serotype or a VSV-G New Jersey serotype.

In matter of vaccination strategy, pseudotyped lentiviral vector particles are more likely to escape the immune system, when this latter already developed immunity against lentiviruses. This is particularly helpful when successive injections of similar particle vectors are required for immunizing a patient against a disease.

The lentiviral vector particle may comprise HIV-1 Gag and Pol proteins, and in particular HIV-1 subtype D Gag and Pol proteins.

A further object of the present invention relates to an isolated cell comprising (i.e. transformed with) a lentiviral vector according to the invention or a lentiviral vector particle of the invention.

A cell according to the invention is preferably a mammalian cell, particularly a human cell. Particularly preferred are human non-dividing cells.

Another object of the present invention relates to a vaccine composition comprising a lentiviral vector according to the invention, a lentiviral vector particle according to the invention or a cell according to the invention.

A vaccine composition according to the invention comprises a pharmaceutically acceptable medium.

By “pharmaceutically acceptable medium” is meant any solution used to solubilize and deliver a lentiviral vector, a lentiviral vector particle or a cell according to the invention to an individual. A desirable pharmaceutically acceptable carrier is saline. In desirable embodiments, a pharmaceutically acceptable medium includes an adjuvant.

Appropriate physiologically acceptable mediums and their formulations are known to one skilled in the art and described, for example, in Remington's Pharmaceutical Sciences, (20th edition), ed. A. Gennaro, 2003, Lippincott Williams & Wilkins.

### Implementations According to the Invention

An object of the present invention relates to a lentiviral vector of the invention, a lentiviral vector particle of the invention or an isolated cell of the invention for use as a medicament or vaccine.

In particular, an object of the present invention relates to a lentiviral vector of the invention, a lentiviral vector particle of the invention or an isolated cell of the invention, in particular in the form of a vaccine composition according to the invention, for use in the treatment or prevention of an HPV induced cancer and metastases thereof, in particular of an HPV-induced cancer.

As previously indicated, HPV induced cancers are cancers induced by an infection by HPV. Methods for the detection of HPV in cancers are known in the art (Aldo Venuti and Francesca Paolini; Head Neck Pathol. 2012 July; 6(Suppl 1): 63-74).

HPV induced cancers can in particular be selected from the group consisting of cervical cancer, vaginal cancer, vulvar cancer, penile cancer, anal cancer and oropharyngeal cancer.

Metastases of such cancers according to the invention may in particular be pulmonary metastasis.

Such prevention and/or treatment implies the administration of the considered active, in particular a vaccine composition of the invention as defined above, to an individual in need thereof.

An individual in need thereof is an animal, in particular a mammal, and may more particularly be a human being.

Lentiviral vectors, lentiviral vector particles, cells and vaccine compositions according to the invention are administered to an individual in need thereof by conventional methods, in dosages which are sufficient to elicit an immunological response, which can be easily determined by those skilled in the art.

Lentiviral vectors, lentiviral vector particles, cells and vaccine compositions according to the invention may accordingly be administered intravenously or intramuscularly as indicted below.

Lentiviral vectors, lentiviral vector particles, cells and vaccine compositions according to the invention may alternatively be administered intranasally. This route of administration is particularly useful in the treatment or prevention of oropharyngeal cancers and/or pulmonary metastases.

Lentiviral vectors, lentiviral vector particles, cells and vaccine compositions according to the invention are administered in a therapeutically effective amount, and may in particular be administered in a dose corresponding to at least 1×10⁶, 2×10⁶, 5×10⁶, 10⁷, 2×10⁷, 5×10⁷, 1×10⁸, 2×10⁸, 5×10⁸, or at least 1×10⁹ TU (Transduction units) of lentiviral vectors according to the invention, in particular in a dose corresponding to at least 1×10⁷, 2×10⁷, 5×10⁷, 1×10⁸ TU or at least 1×10⁹ TU of lentiviral vectors according to the invention. In a preferred embodiment, the lentiviral vectors, lentiviral vector particles, cells and vaccine compositions according to the invention are administered in a dose corresponding to at least 1×10⁷ TU of lentiviral vectors according to the invention, more particularly at least 1×10⁸ TU of lentiviral vectors according to the invention and in particular at least 1×10⁹ TU of lentiviral vectors according to the invention.

By a “therapeutically effective amount” is for example meant the amount of a lentiviral vector or lentiviral vector particle, cell or vaccine composition according to the invention required to generate in a subject one or more of the following effects: an immune response against an HPV induced tumor; a decrease in the size of the HPV induced tumor, i.e. a reduction of at least 5%, 10%, 20%, 30%, 40%, 50%, 60%, 70%, 80%, 90%, or of 100% of the tumor size in 15 to 45 days compared to the size of the tumor at the time of administration); an increase in the CD8+ and/or CD4+ infiltration in the HPV induced tumor in 5 to 45 days following the administration; a decrease of CD25+FoxP3+CD4+ Regulatory T cells (Tregs) in the HPV induced tumor in 5 to 45 days following the administration.

Administration can be performed using well known routes including, for example, intravenous, intramuscular, intranasal, intraperitoneal or sub-cutaneous injection, and in particular intravenous, intranasal or intramuscular, and may be intravenous or intramuscular.

The appropriate dose and regimen will obviously vary between species and individuals depending on many factors. For example, higher doses will generally be required for an effective immune response in a human compared with a mouse.

Lentiviral vectors, lentiviral vector particles, cells and vaccine compositions according to the invention may for example be administered in a single dose, as illustrated in the examples, or in two or more administrations. Practitioners will determine, in each case, the appropriate regimen and dosage for the administration of actives according to the invention.

Lentiviral vectors, lentiviral vector particles, cells and vaccine compositions according to the invention may advantageously be administered in combination with at least one immune checkpoint inhibitor (ICI).

An immune checkpoint inhibitor (ICI) according to the invention may in particular be an antibody, in particular an anti-PD-1, an anti-PD-L1 (PD-1 Ligand), an anti-CTLA-4 (Cytotoxic T-Lymphocyte-Associated protein 4), an anti-NKG2A, an anti-TIM-3 (T-cell immunoglobulin and mucin-domain containing-3), an anti-TIGIT (T cell immunoreceptor with Ig and ITIM domains) or an anti-LAG-3 (Lymphocyte-activation gene 3) antibody. More particularly, the at least one immune checkpoint inhibitor according to the invention may be a monoclonal antibody selected from the group consisting of anti-PD-1, anti-PD-L1, anti-CTLA-4, anti-NKG2A, anti-TIM-3, anti-TIGIT and anti-LAG-3 monoclonal antibodies. Even more particularly, the at least one immune checkpoint inhibitor according to the invention may be a monoclonal antibody selected from the group consisting of anti-PD-1, anti-PD-L1, anti-CTLA-4, anti-NKG2A, anti-TIM-3 and anti-TIGIT monoclonal antibodies.

An immune checkpoint inhibitor (ICI) according to the invention may more particularly be an antibody, in particular an anti-PD-1, an anti-PD-L1 (PD-1 Ligand), an anti-CTLA-4 (Cytotoxic T-Lymphocyte-Associated protein 4), an anti-NKG2A, an anti-TIM-3 (T-cell immunoglobulin and mucin-domain containing-3), an anti-TIGIT (T cell immunoreceptor with Ig and ITIM domains) or an anti-LAG-3 (Lymphocyte-activation gene 3) antibody and even more particularly be an antibody, in particular an anti-PD-1, an anti-PD-L1 (PD-1 Ligand), an anti-CTLA-4 (Cytotoxic T-Lymphocyte-Associated protein 4), an anti-NKG2A, an anti-TIM-3 (T-cell immunoglobulin and mucin-domain containing-3) or an anti-TIGIT (T cell immunoreceptor with Ig and ITIM domains) antibody. More particularly, the at least one immune checkpoint inhibitor according to the invention may be a monoclonal antibody selected from the group consisting of anti-PD-1, anti-PD-L1, anti-CTLA-4, anti-NKG2A, anti-TIM-3, anti-TIGIT and anti-LAG-3 monoclonal antibodies and in particular may be a monoclonal antibody selected from the group consisting of anti-PD-1, anti-PD-L1, anti-CTLA-4, anti-NKG2A, anti-TIM-3 and anti-TIGIT monoclonal antibodies.

An anti-PD-1 monoclonal antibody may for example be selected from the group consisting of Nivolumab, Pembrolizumab and Cemiplimab.

An anti-PD-L1 monoclonal antibody may for example be selected from the group consisting of Atezolizumab, Avelumab and Durvalumab.

An anti-CTLA-4 monoclonal antibody may for example be selected from the group consisting of ipilimumab, tremelimumab and quavonlimab.

An anti-NKG2A monoclonal antibody may for example be monalizumab.

An anti-TIM-3 monoclonal antibody may for example be selected from the group consisting of Sym023 and sabatolimab.

An anti-TIGIT monoclonal antibody may for example be tiragolumab.

An anti-LAG-3 monoclonal antibody may for example be relatlimab.

In particular, the ICI may be an anti-PD-L1 or an anti-PD-1 monoclonal antibody, and in particular be an anti-PD-1 monoclonal antibody.

The vaccine composition, lentiviral vector, lentiviral vector particle or cell for use according to the invention and the immune checkpoint inhibitor may be administered simultaneously or separately.

Considering the unexpected synergistic advantageous properties obtained when combining a lentiviral vector according to the invention with an immune checkpoint inhibitor as demonstrated in the examples, it can be anticipated that vaccination with a lentiviral vector according to the invention may increase the number of patients eligible for immune checkpoint inhibitor therapy, especially anti-PD-1.

By simultaneously, it is understood that (i) the vaccine composition, lentiviral vector, lentiviral vector particle or cell and (ii) the immune checkpoint inhibitor, may be administered at the same moment or up to the same day or couple of days. In this case, they can be administered in the same composition or in separate compositions.

By separately, it is understood that (i) the vaccine composition, lentiviral vector, lentiviral vector particle or cell according to the invention and (ii) the immune checkpoint inhibitor may be administered with at least several days, for example at least two days of difference.

In particular, when (i) the vaccine composition, lentiviral vector, lentiviral vector particle or cell and (ii) the immune checkpoint inhibitor are administered separately, the vaccine composition, lentiviral vector, lentiviral vector particle or cell according to the invention may be administered before the immune checkpoint inhibitor.

Advantageously, the vaccine composition, lentiviral vector, lentiviral vector particle or cell according to the invention may be administered at least 2 and in particular at least 4 days before the administration of the immune checkpoint inhibitor. Accordingly, the immune checkpoint inhibitor may advantageously be administered at least 2 and in particular at least 4 days after the vaccine composition, lentiviral vector, lentiviral vector particle or cell according to the invention. The immune checkpoint inhibitor may more particularly be administered 4 days to 1 month after the vaccine composition, lentiviral vector, lentiviral vector particle or cell according to the invention, in particular 4 days to 15 days, and more particularly 4 days to 10 days after the vaccine composition, lentiviral vector, lentiviral vector particle or cell according to the invention.

The vaccine composition, lentiviral vector, lentiviral vector particle or cell for use according to the invention and the immune checkpoint inhibitor may be administered by the same route or through different routes.

The at least one immune checkpoint inhibitor herein is administered in a therapeutically effective dose, i.e. a dose that produces the effects for which it is administered. The exact dose of immune checkpoint inhibitor will depend on the purpose of the treatment and will be ascertainable by one skilled in the art using known techniques.

The invention further relates to a method for the treatment and/or prevention of an HPV induced cancer in an individual in need thereof, comprising the administration to said individual of at least one lentiviral vector of the invention, lentiviral vector particle of the invention or isolated cell of the invention, in particular in the form of a vaccine composition according to the invention.

The invention further relates to the use of at least one lentiviral vector of the invention, lentiviral vector particle of the invention or isolated cell of the invention, in particular in the form of a vaccine composition according to the invention for the treatment and/or prevention of an HPV induced cancer in an individual in need thereof.

The examples and figures which follow are presented by way of illustration and without implied limitation of the invention.

### EXAMPLES

### Materials and Methods

### Mice

C57BL6jRj mice were purchased from Janvier Labs (Le Genest-Saint-Isle, France). All animals were maintained under specific pathogen-free conditions, and all procedures were performed according to an approved animal protocol and in accordance with recommendations for the proper use and care of laboratory animals. All animal experiments were conducted in accordance with guidelines established by the French and European regulations for the care and use of laboratory animal.

### Peptides, Antibodies, and Reagents

To test reactivity of the vaccines according to the invention, 15mer overlapping peptides were ordered from GenScript Biotech (Netherlands) at a purity of >80%. Anti CD4-VioBlue (Clone REA604), anti-CD45-VioGreen (Clone REA737), Anti-FoxP3-Vio515 (clone REA788), anti-CD279 (PD1)-PE (clone REA802), anti-CD8a-PE-Vio770 (clone REA601), antiCD25-APC (clone REA568), anti CD11c-FITC (clone REA754), anti CD11b-APC-Vio770 (clone REA592) were purchased from Miltenyi Biotec. Anti anti-mouse H-2kb (Clone AF6-88.5), anti-CD274 (PD-L1)-APC (Clone MIH5) and anti CD16/CD32 (Clone 2.4G2) were purchased from BD Biosciences.

Antibodies were mixed together with PBS containing 1% FCS (Gibco).

Cyclophosphamide was purchased from Sigma, resuspended in PBS (Gibco) and stored at −20° C. before use.

### Cells

HPV-16 E6 and E7-expressing TC-1 tumor cells were generated as previously described (Lin et al. Cancer Res. 1996 Jan. 1; 56(1):21-6): Primary lung cells of C57BL6 mice were transformed with HPV-16 E6 and E7 genes and with pVEJB-expressing activated human c-Ha-ras oncogene. TC-1 cell line was cultured in Glutamax RPMI medium (Gibco supplemented with 100 U/ml penicillin, 100 μg/ml streptomycin, and 10% fetal bovine serum).

### Lentiviral Vector Construction

The antigen (Ag) constructs were cloned in a pFlap-B2m-Ag-WPREMutee backbone (see for example WO2016012623 for the backbone). The antigen plasmid contains the cPPT/CTS sequence (sequence SEQ ID NO: 37), mandatory for transduction of non-mitotic cells. The U3 promoter sequence was deleted from the 3′ long terminal repeat (LTR) to avoid vector replication. The Beta-2microglobuline (β2m) promoter controls vaccine antigen expression in all transduced cells, thereby, antigen will preferentially be expressed in APC (Antigen Presenting Cells). In addition, it is devoid of any known enhancer sequence, likely to induce mutagenesis and/or genotoxic effects. The antigen plasmid contains a mutant form of the woodchuck hepatitis B virus (WHV) post-transcriptional regulatory element (WPRE) (sequence SEQ ID NO: 38). The wild-type WPRE region contains a truncated form of the WHV X protein that may have oncogenic properties (Kingsman et al., Gene Ther. 2005 January; 12(1):3-4). The mutant form of the WPRE used in our construct precludes expression of the truncated X protein by the inclusion of point mutations within the X protein start codon. Such mutant WPRE sequence appears not to have oncogenic properties (Themis et al., Mol Ther. 2005 October; 12(4):763-71).

The packaging plasmid (pNDK) contains the gag-pol sequences from HIV-1 subtype NDK (GenBank acc n°: A34828). The proteins nef, vif, vpr, env are not expressed. Moreover, aspartic acid (D) to valine (V) replacement at position 64 (D64V) in the HIV-1 integrase protein sequence (pol gene) is sufficient to inhibit integration without disturbing the transgene expression in vitro. The lentiviral particles according to the invention are non-integrative particles.

Envelope plasmid: pCMV-VSV-G INDco (Indiana) et pCMV-VSV-G NJco (New Jersey) vectors were constructed by subcloning the Vesicular stomatitis virus (VSV) G protein (VSV-G) Indiana (GenBank acc. n^{o} J02428) and New Jersey (GenBank acc. n° P04882) serotype inserts into the pVAX1 expression vector (Invitrogen). Mammalian codon-optimized synthetic genes (GeneArt) encoding glycoproteins from the following Vesiculovirus were cloned into a pVAX1 plasmid (Invitrogen): Vesicular Stomatitis Virus Indiana serotype (GenBank FW591952), New Jersey serotype (GenBank FW591956) and Cocal virus (GenBank: AF045556.1).

### Lentiviral Vector Particles Production

After amplification of HEK 293 T cells (ATCC) in DMEM with 1% penicillin/streptomycin and 10% FCS, non integrative lentiviral particles were produced by transient calcium phosphate co-transfection of HEK 293 T cells (ATCC) with 3 plasmids (The viral antigen plasmid, an envelope expression plasmid and packaging plasmid) following the method well-known in the art. Culture medium is replaced by serum free medium after 24 h. Supernatant is harvested and clarified 48 h after transfection by 2500 rpm centrifugation. Viral particles are concentrated by ultracentrifugation (1 h at 22000 rpm/88250 g 4oC) and resuspended in preservative buffer (20 mM Pipes, 75 mM NaCl and 2.5% sucrose).

### Vector Titration

Lentiviral vector titer was determined by quantitative PCR after transduction of cells (HEK 293 T). Aphidicolin is added to HEK293T cells 24 h before transduction and is maintained during the whole titration process. Cells are incubated 30 min with lysis buffer (200 mM Tris, 1% NP40 and 1% Tween20), containing 50 μg/ml RNase A (sigma). Proteinase K (0.2 mg/ml) is added to suspension and incubated 4 h at 56° C. Pairs of primers specific for RRE (element of Ag vector) and GAPDH (in host cell) are used for quantitative PCR. Titer are Titer are expressed as transduction unit (TU)/mL of vector.

### Design of HPV Vaccine

The implemented non-oncogenic immunogenic E6 and E7 protein sequences were selected and modified as previously discussed herein.

In particular, 4 different vaccines were designed comprising the following lentiviral vectors:
the lentiviral vector filed at the Collection Nationale de Cultures de Microorganismes (CNCM) on Oct. 21, 2021 under accession number I-5759;
the lentiviral vector filed at the Collection Nationale de Cultures de Microorganismes (CNCM) on Oct. 21, 2021 under accession number I-5760;
the lentiviral vector filed at the Collection Nationale de Cultures de Microorganismes (CNCM) on Oct. 21, 2021 under accession number I-5761; or
the lentiviral vector filed at the Collection Nationale de Cultures de Microorganismes (CNCM) on Oct. 21, 2021 under accession number T-5762.

These lentiviral vector particles comprising these functionals lentivirals vectors quantified by vector titration and expressed as transduction units (TU) were implemented in the following examples.

### In Vivo Immunogenicity of Lentiviral Vector Vaccine (LV Vaccine)

Naïve C57BL6 female mice were vaccinated with a LV vaccine according to the invention (i.e. lentiviral vector particles of the invention comprising functional lentiviral vector of the invention) via intramuscular (i.m.) injection in 50 μL of diluent. 14 days later, splenocytes were prepared and restimulated overnight for IFNg ELISPOT with 4 distinct HPV peptide pools (each peptide is 2 μg/mL final). Each peptide pools correspond to one of the following non-oncogenic antigen variants: non-oncogenic variant of E6 protein of HPV16, non-oncogenic variant of E7 protein of HPV16, non-oncogenic variant of E6 protein of HPV17 and non-oncogenic variant of E7 protein of HPV17. They are composed of overlapant 15 mers (with overlaps of 11 a.a.) corresponding to the full selected antigen.

### In Vivo Tumor Vaccination Treatment

For in vivo tumor experiments, 1·10⁶ TC-1 cells were injected into 7- to 9-week-old C57BL/6 mice subcutaneously (s.c.) in the right flank (mice were shaved with electric shaver device before injection). When average tumor volume reaches expected range, mice were randomized and injected with the LV vaccine of the invention via intramuscular (i.m.) injection. Mice were monitored for tumor growth by measuring tumor diameter with calipers 3 times a week. Due to ethical reasons, mice with tumors >1500 mm3 had to be euthanized.

### Immunogenicity of LV Vaccine of the Invention in Human PBMC

Frozen human PBMC (StemCell) were gently thawed, stained during 10 min at 37° C. with 0.5 μM of CFSE (Thermofischer). Cells were then cultured in round bottom 96 wells plate (0.2×106 cells per well) in complete RPMI: 10% FCS, 10 mM Hepes (Gibco), 100 U/ml Penicillin, 100 μg/ml Streptomycin, 0.1 mM Non-Essential Amino acids (Gibco) and 1 mM Sodium Pyruvate (Gibco). After 7 days, cells are centrifuged and new complete RPMI (prewarmed) is added. After another 7 days (14 days total), cells are stained with fluorescent antibodies and data are acquired by flow cytometry (MACSQuant analyzer)

### Cytometric Analysis of Tumor Immune Infiltrates

Tumors were treated with the Mouse Tumor Dissociation kit (Miltenyi). Cell suspensions were then filtered through 70 m-pore filters, treated with Red Blood Cell lysis buffer (Sigma), then washed and centrifuged at 1200 rpm for 5 minutes. The recovered cells were stained as follows.

To detect NK, Near IR LD (Invitrogen), FcγII/III receptor blocking anti-CD16/CD32 (clone 2.4G2, BD Biosciences), APC-anti-CD11b (clone N418, BD Biosciences), BV421-anti-NKp46 (clone 29A1.4, Biolegend) were used.

Samples were acquired in an Attune NxT cytometer (Invitrogen) and data analyzed by FlowJo software (Treestar, OR, USA).

### Intracellular Cytokine Staining

Splenocytes from immunized mice were obtained by tissue homogenization and passage through 100-μm nylon filters (Cell Strainer, BD Biosciences) and were plated at 4×10⁶ cells/well in 24-well plates. Splenocytes were stimulated during 6 h in the presence of 10 μg/mL of homologous or control peptide, 1 μg/mL of anti-CD28 (clone 37.51) and 1 μg/mL of anti-CD49d (clone 9C10-MFR4.B) mAbs (BD Biosciences). During the last 3 h of incubation, cells were treated with a mixture of Golgi Plug and Golgi Stop, both from BD Biosciences. PE-Cy7-anti-CD107a (clone 1D4B, BioLegend) mAb was also added to the cultures at this step. Cells were then collected, washed with PBS containing 3% FBS and 0.1% NaN3 (FACS buffer) and incubated for 25 min at 4° C. with a mixture of Near IR Live/Dead (Invitrogen), FcγII/III receptor blocking anti-CD16/CD32 (clone 2.4G2), PerCP-Cy5.5-anti-CD3ε (clone 145-2C11), PE-Cy7-anti-CD4 (clone RM4-5) and BV711-anti-CD8 (clone 53-6.7) mAbs (BD Biosciences or eBioscience). Cells were washed twice in FACS buffer, then permeabilized by use of Cytofix/Cytoperm kit (BD Bioscience). Cells were then washed twice with PermWash 1× buffer from the Cytofix/Cytoperm kit and incubated with a mixture of BV421-anti-IL-2 (clone JES6-5H4), FITC-anti-TNF (MP6-XT22), APC-anti-IFN-7 (clone XMG1.2) and BV605-anti-IL-17A (Clone TC11-18H10) mAbs (BD Biosciences) or a mixture of appropriate control Ig isotypes, during 30 min at 4° C. Cells were then washed twice in PermWash and once in FACS buffer, then fixed with Cytofix (BD Biosciences) overnight at 4° C. Cells were acquired in an Attune NxT cytometer system (Invitrogen) and data analysis was performed using FlowJo software (Treestar, OR, USA).

### Example 1: HPV Vaccines of the Invention are Immunogenic In Vivo

In order to measure the capacity of vaccines of the invention to induce an immune response, recipient mice were immunized with the 4 vaccines (1 group of 5 mice per tested vaccine and control group).

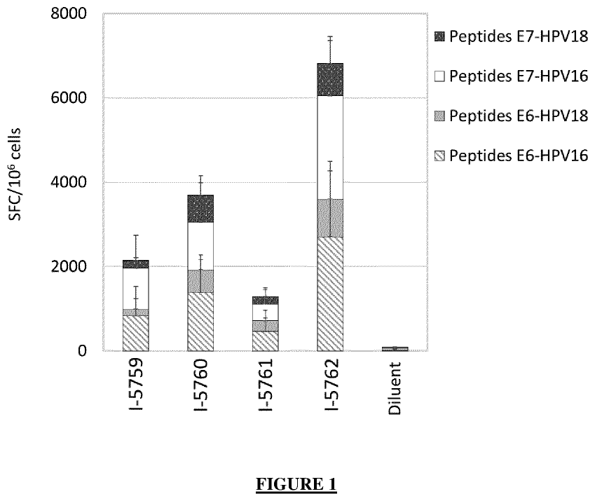

Mice were injected i.m. with 1×10⁷ TU of lentiviral vector particles comprising the lentiviral vector filed at the CNCM under accession number I-5759, the lentiviral vector filed at the CNCM under accession number I-5760, the lentiviral vector filed at the CNCM under accession number I-5761 or the lentiviral vector filed at the CNCM under accession number I-5762 or 50 μL of diluent. 14 days later, splenocytes were prepared and restimulated overnight for IFNg ELISPOT with 4 distinct peptide pools (each peptide is 2 μg/mL final). The results obtained are represented in FIG. 1.

### Example 2: HPV Vaccines of the Invention Vaccine Fully Eliminates Well Implanted Tumors In Vivo

TC-1 tumors cells have been extensively used as a preclinical model to study HPV induced Tumors (Kim, J W et al. Gene therapy vol. 11, 12 (2004): 1011-8). These lung tumor cells were modified to express E6 and E7 from HPV16 (Lin et al. Cancer Res. 1996 Jan. 1; 56(1):21-6).

After s.c. (subcutaneous) injection of TC-1 cells to the mice, solid tumors are rapidly found at injection site and untreated animal tumors grow to reach ethical endpoint in 30-40 days. In order to test the efficacy of lentiviral vector particles of the invention, TC-1 cells were injected s.c. and tumor volume was measured every other day (caliper measurement). When average tumor volume is 70 mm³, mice were randomized and vaccinated with 1×10⁸ TU i.m. of LV-GFP Indiana (as a control), Indiana lentiviral vector particles comprising I-5759, Indiana lentiviral vector particles comprising I-5760, Indiana lentiviral vector particles comprising I-5761 or Indiana lentiviral vector particles comprising I-5762.

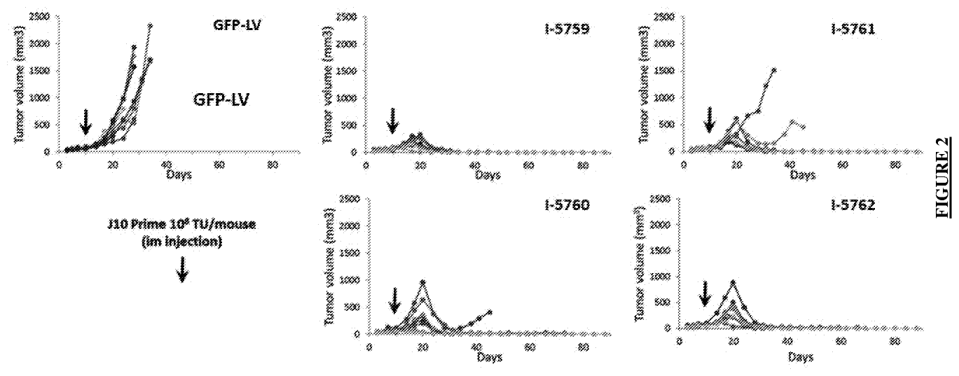

The results obtained are represented in FIG. 2.

A rapid and very efficient elimination of tumors is observed in 100% of animals vaccinated with lentiviral vector particles comprising the lentiviral vector filed at the CNCM under accession number I-5762 and the lentiviral vector filed at the CNCM under accession number T-5759, 87.5% of animals vaccinated with the lentiviral vector filed at the CNCM under accession number I-5760 and 75% of animals vaccinated with the lentiviral vector filed at the CNCM under accession number I-5761.

Vaccines comprising lentiviral vector particles comprising the lentiviral vector filed at the CNCM under accession number I-5759 and lentiviral vector particles comprising the lentiviral vector filed at the CNCM under accession number I-5762 show equivalent tumor elimination rates (higher than I-5760 and I-5761) but I-5759 vaccination allows full elimination of tumors in 37.5 days (+/−7.4 SD) on average whereas it takes 54.7 days after I-5762 vaccination.

We surprisingly observed that the most immunogenic vector was not the most protective and that the ranking in immunogenicity did not apply for anti-tumor efficacy. Indeed, the lentiviral vector particle comprising the lentiviral vector filed at the CNCM under accession number I-5759 was the most efficient anti tumoral vaccine whereas the lentiviral vector particles comprising the lentiviral vector filed at the CNCM under accession number I-5760 and I-5762 were more immunogenic vectors when measuring IFN-γ production.

### Example 3: A Single HPV Vaccine of the Invention Administration is Efficient Against Tumor Relapse

Relapse is commonly observed in most cancer type and is defined as a return of the disease after a period of improvement. It is often due to a few tumor cells that survived the initial treatment and form new tumors weeks, month or even years after treatment.

A. In order to mimic a relapse in our model, mice who eliminated primary tumor were rechallenged on the other flank at day 60. Control mice (untreated) were also injected s.c. in order to check on the tumor cell injection.

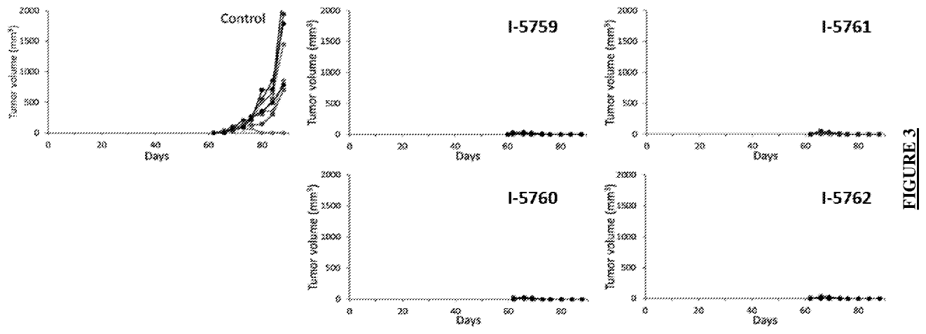

The results obtained are represented in FIG. 3.

This Figure shows that s.c. injection of TC-1 cells in control mice allows formation of solid tumors reaching ethical limits of size in less than 30 days. Tumor growth in rechallenged mice (that previously eliminated right flank tumors after vaccination) were strongly reduced. Tumor growth was observed during the first 6 days and tumor elimination begins afterwards.

Tumors are even fully eliminated 13-16 days after implantation. Single dose vaccination administered on tumor bearing mice allowed full elimination of primary tumor and a strong protection against relapse as these mice rapidly eliminated secondary tumors.

B. A additional experiment was performed with a rechallenge of the mice who eliminated primary tumor on the other flank with 1·10⁶ TC-1 tumor cells 119 days after the first engraftment and were maintained without any therapy. Control mice (untreated) were also injected s.c. in order to check on the tumor cell injection.

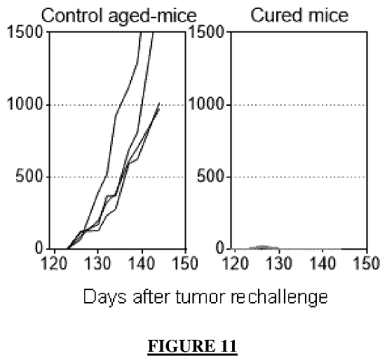

The results obtained are represented in FIG. 11.

All re-challenged mice were still alive 145 days after the initial tumor challenge, sustaining that a single injection of a vaccine according to the invention effectively promoted a strong antitumor memory protective immune response which efficiently shaped T-cell responses to new challenges.

### Example 4: Therapeutic Effect of Anti HPV Vaccine of the Invention is Dose Dependent

In order to determine the effect of a lower dose of vaccine, an efficacy study was performed on mice bearing TC1 tumor. Mice were vaccinated with the vaccine according to the invention comprising lentiviral vector particles comprising the lentiviral vector filed at the CNCM under accession number I-5759, at 1×10⁸ or 1×10⁷ TU/mouse.

In particular, 1×10⁶ TC-1 cells were injected in the flank of animals and tumor volume was measured twice a week (caliper measurement). When average tumor volume was 80 mm3, mice were randomized and vaccinated with diluent (control), 1×10⁷ TU or 1×10⁸ TU (i.m.) of I-5759 vaccine

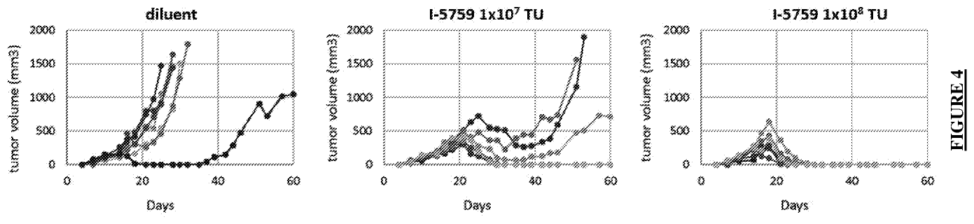

The results obtained are represented in FIG. 4.

Whereas vaccination with 1×10⁸ TU allows total and rapid elimination of tumors (in less than 20 days after vaccination), we observed that 1×10⁷ TU vaccination dose had a partial effect on tumor growth. 3/6 (50%) mice were tumor free 22 days after vaccination, and the others first showed a decline 15-18 days post vaccination (during 5-10 days) but tumor growth couldn't be controlled afterwards.

A single low dose (1×10⁷) of a vaccine according to the invention showed a partial inhibition comparable to what is observed with 3 injections of Adenoviral vector based vaccines (Rice, A E et al. Cancer gene therapy vol. 22, 9 (2015): 454-62). This suggests that a vaccine according to the invention at optimal dose would be more efficient than adenoviral platform. Moreover, low dose efficacy would most likely be increased by a second injection of vaccine.

### Example 5: Vaccination According to the Invention Increases CD4⁺ and CD8⁺ T Cell Infiltration and Reduces T Reg in the Treated Tumors

Tumor infiltration was investigated to understand further the anti-tumoral mechanisms induced after vaccination with a lentiviral vector according to the invention. 1×10⁶ TC1 tumor cells were injected (s.c.) on the flank of animal, and the tumor volume was measured twice a week (caliper measurement). When average tumor volume was 80 mm³, mice were randomized and vaccinated with either diluent (control), or 1×10⁷ TU of I-5759 or 1×10⁸ TU (i.m.) of I-5759.

Ten days after vaccination, tumors were collected, digested and analyzed by flow cytometry. FACS staining was performed and data were acquired on Macsquant facs according to methods well known in the art.

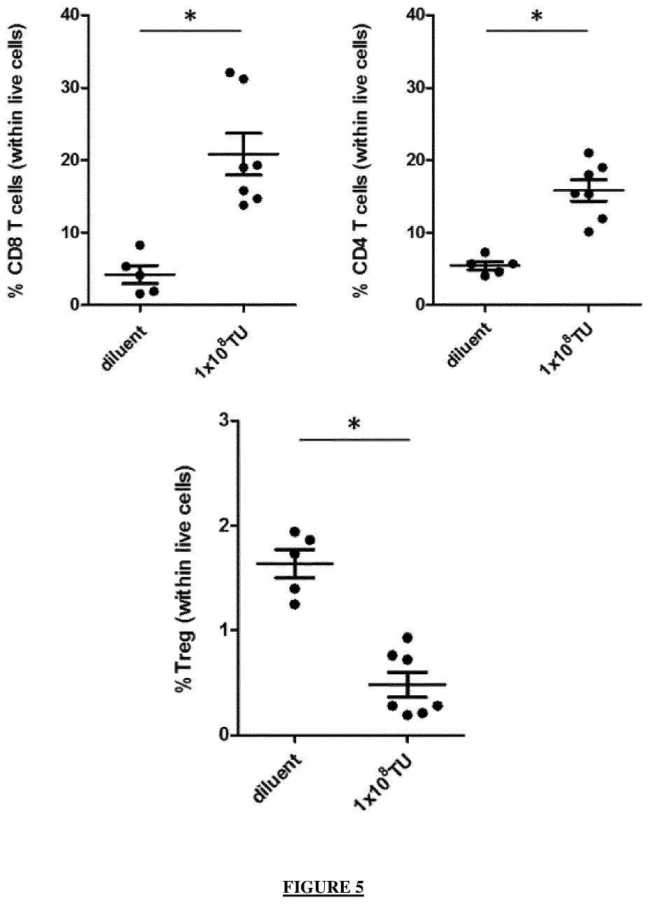

The results obtained are represented in FIG. 5.

Tumors from vaccinated mice were infiltrated with more CD8⁺ and CD4⁺ T cells compared to control tumors. The percentage of CD8⁺ T cells and CD4⁺ T cell in the tumors are increased respectively by about 4 and 3 times. On the other hand, the percentage of CD25+FoxP3+CD4+ Regulatory T cells (Tregs) in tumors is strongly reduced in treated animals.

These observations are of major importance as they suggest that vaccines comprising lentiviral vectors of the invention improve CD8+ T cells and CD4+ T cell recruitment to the tumor but also reduces the percentage of Tregs in the tumors.

### Example 6: HPV Vaccine of the Invention Fully Eliminates Large Tumors In Vivo

Well established tumors are known to be more difficult to eliminate than small and early-stage tumors. Most vaccines tested on TC1 model have weaker effect when administered at latter time point (Rice, A E et al. Cancer gene therapy vol. 22, 9 (2015): 454-62; Berraondo, Pedro et al. Cancer research vol. 67, 18 (2007): 8847-55). 1×10⁶ TC1 tumors were injected (s.c.) on the flank of animal. When average tumor volume was around 300 mm³, mice were randomized and vaccinated with diluent (control), or 1×10⁸ TU (i.m.) of the vaccine according to the invention comprising lentiviral vector particles comprising the lentiviral vector filed at the CNCM under accession number I-5759. Tumor volume was measured twice a week with a caliper.

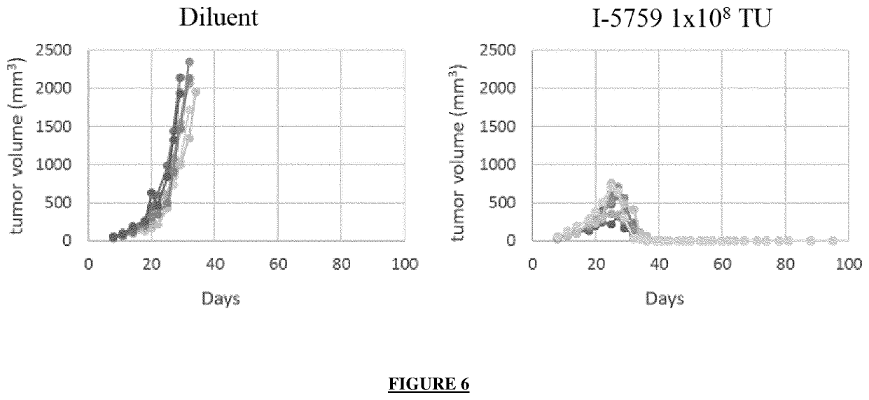

The results obtained are represented in FIG. 6.

It can be seen that a vaccine according to the invention is highly efficient in completely eliminating well-established HPV induced tumors.

### Example 7: HPV Vaccine of the Invention Induces Human PBMC Activation In Vitro

In order to verify that a vaccine according to the invention can induce a T cell response in human cells, human PBMC (StemCell) were labelled with CFSE and cultured in absence (unstimulated condition) or presence of a vaccine according to the invention (I-5759). Cell proliferation and activation were measured after 2 weeks of culture.

CD8⁺ T cells and CD4⁺ T cells proliferation (measured by CFSE dilution) and expression of CD25 activation marker were increased by addition of lentiviral vectors of the invention in the culture.

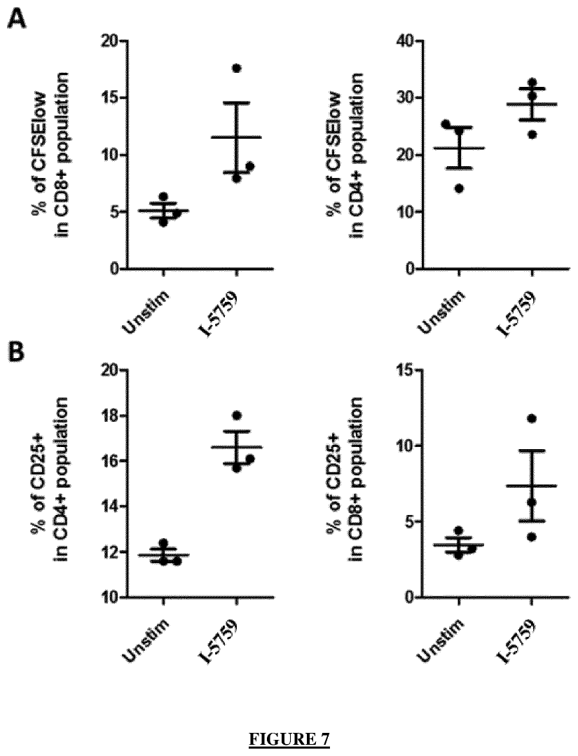

The results obtained are represented in FIG. 7.

It can thereby be concluded that antigen presenting cells from the PBMC are able to be transduced by an HPV vaccine according to the invention and to process the antigens to activate T cells.

### Example 8: Systemic T-Cell Immunity Induced by HPV Vaccine of the Invention and Phenotype of Effector T Cells

To gain further insight into the quality of the T-cell responses induced, splenocytes from mice injected with a Ctrl Lenti (LV-empty Indiana) or with a vaccine according to the invention comprising lentiviral vector particles comprising the lentiviral vector filed at the CNCM under accession number I-5759 were left untreated or were stimulated in vitro with a mixture of ETTDPDRAHYNIVTF and PDRAHYNIVTFCCKC E7_{HPV16}-derived peptides which contain the immunodominant H-2Db-restricted RAHYNIVTF epitope (Feltkamp M C et al., Eur J Immunol 1993; 23:2242-9) and were analyzed by IntraCellular Staining (ICS) for IL-2, and TNF-α and IFN-7.

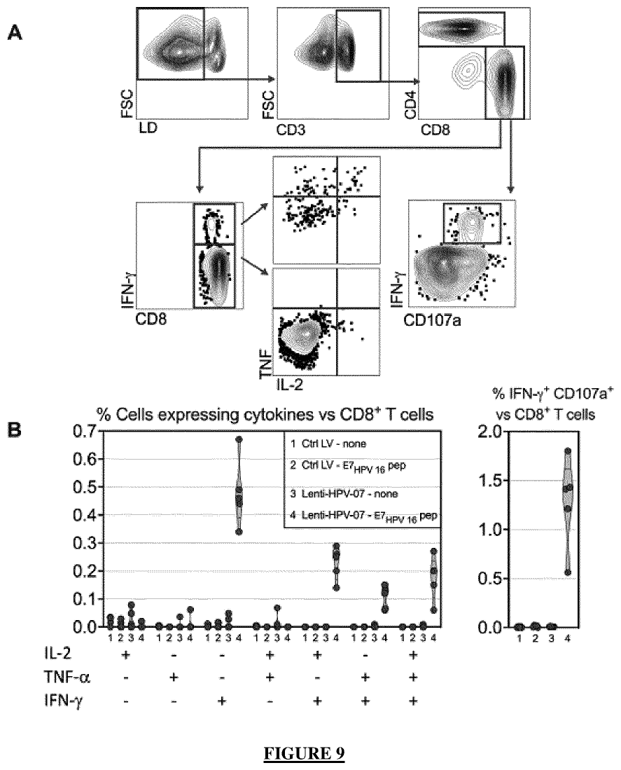

The results obtained are represented in FIG. 9A.

Stimulation with these peptides detected CD8⁺ T-cell responses, in mice vaccinated with a lentiviral vector according to the invention. The functional CD8⁺ T-cell effectors were mainly distributed among IFN-γ⁺ (single positive), TNF-α⁺ IFN-γ⁺ or IL-2⁺ IFN-γ⁺ (double positive), and IL-2⁺ TNF-α⁺ IFN-γ⁺ (triple positive) subsets (voir FIG. 9B).

The majority of IFN-γ⁺ CD8⁺ T cells also expressed the surface CD107a degranulating marker, showing the effector properties of these T cells (voir FIG. 9B).

### Example 9: Features of Tumor Cells and Tumor Infiltrating Innate Immune Cells in Mice Vaccinated with an HPV Vaccine of the Invention

Characterization by cytometry of the intra-tumoral infiltrates at day 11 post-vaccination and thus during the tumor regression phase showed a significant increase in the proportion of Natural Killer (NK) cells in the regressing tumors from mice vaccinated with a vaccine according to the invention comprising lentiviral vector particles comprising the lentiviral vector filed at the CNCM under accession number I-5759.

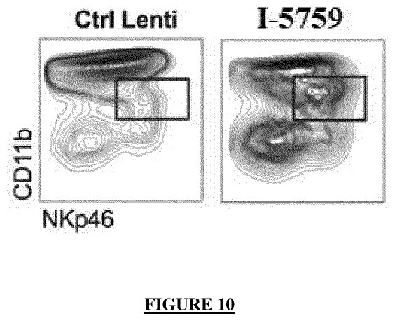

Results are shown in FIG. 10.

### Example 10: Suboptimal Lenti-HPV-07 Vaccination Acts Synergistically with Anti-PD1 Immunotherapy

The inventors further investigated the potential synergy between a suboptimal dose of Lenti-HPV-07 (I-5759) vaccination and anti-PD-1 therapy.

The anti-PD-1 treatment began four days (D17, i.e. 17 days after s.c. administration of the TC-1 cells to the mice) after the injection of the vaccine according to the invention comprising lentiviral vector particles comprising the lentiviral vector filed at the CNCM under accession number I-5759 (D13, i.e. 13 days after s.c. administration of the TC-1 cells to the mice). Several injections of anti-PD-1 were performed (D17 as mentioned above, then D20, D22, D24, D28 and D31).

In particular, experiments were performed on three identical groups of tumor-engrafted mice. In the first group (10 mice—group control), the mice were administered a LV-empty Indiana (D13) (as a control) and four days later with an anti-PD-1 monoclonal antibody (D17, then D20, D22, D24, D28 and D31). In the second group (12 mice—group control), the mice were administered a vaccine according to the invention (I-5759) (D13) and four days later with a control antibody (isotype ctrl) (D17, then D20, D22, D24, D28 and D31). In the third group (14 mice), the mice were administered a vaccine according to the invention (D13) (I-5759) and four days later with a mAb anti-PD-1 (D17, then D20, D22, D24, D28 and D31).

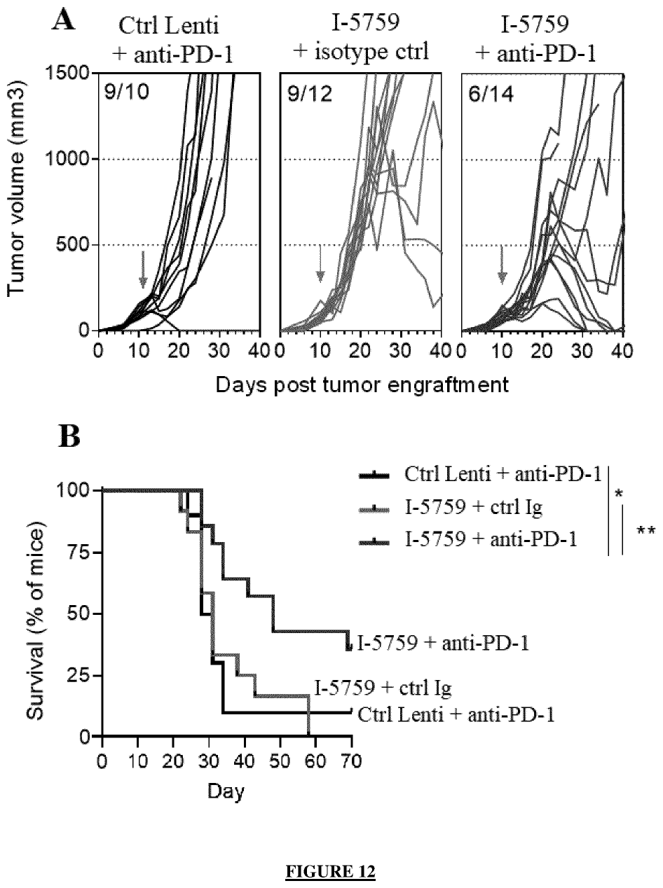

The results are presented in FIGS. 12A and 12B.

A suboptimal dose of the vaccine, which induces an insufficient antitumor T-cell response, acted synergistically with anti-PD-1 to increase the tumor regression rate.

Six mice out of 14 achieved a complete tumor regression, and 2 others showed a partial tumor regression. In the latter, tumor volume decreased by 67% and then the tumor relapsed 6 to 7 days after the end of anti-PD-1 treatment, highlighting the need for repeated injections of anti-PD-1 until the tumor has completely disappeared. Only 3 out of 12 mice treated with the suboptimal dose of vaccine of the invention alone showed partial tumor regression. Accordingly, mice survival was significantly increased in the group of combo treatment, compared to the mice treated with suboptimal dose of Lenti-HPV-07 (FIG. 12B). Therefore, a synergistic anti-tumor effect can be achieved when Lenti-HPV-07 vaccine candidate is combined with the anti-PD1 checkpoint inhibitory treatment.

### Example 11: A Single Injection of Vaccine According to the Invention Cures Mice with Pulmonary Metastatic Foci Induced by Intravenous Injection of TC1-nLuc Cells

TC1 parental cell line was stably transduced with an integrative lentiviral vector encoding for nanoluciferase reporter and puromycin N-acetyl-transferase (for selection), under the ubiquitin (UBC) promoter. After selection on puromycin, cells were subcloned to obtain TC1-nLuc cell line.

Six-week-old C57BL/6JRj mice, purchased from Janvier Laboratory, were intravenously injected with 150 000 TC1-nLuc cells. At day 5, mice were injected with a single dose of 1·10⁹ TU/mouse of Lenti-HPV-07 or Control Lenti (empty vector) via intramuscular route.

Bioluminescence imaging on live animals was performed using the IVIS Imaging System (IVIS Spectrum, Perkin Elmer) coupled to a charged-couple device camera. Prior to bioluminescence imaging, mice were anesthetized with 2% isoflurane in oxygen and maintained in a control flow of 1.5% isoflurane in oxygen through a nose cone during imaging. The substrate furimazine (Z108) (provided by Dr. Yves Janin, Institut Pasteur) was dissolved at 2 mg/ml in acidic ethanol. Z108 was further diluted in sterile D-PBS to the desired concentration (0.4 mg/kg) prior to intravenous injection. Mice were then immediately placed in the imaging chamber and imaged. Sequential images were captured under the auto-exposure settings with a maximum exposure time of 2 minutes.

Images from each experimental set were analyzed using Living Image Software (Ver. 2.50.1 Xenogen). Measurements from regions of interest were performed and the luminescence values were evaluated as total flux (photons/second). Mice bellies and torsos were shaved to enhance signals to noise ratio. The baseline signals were obtained from untreated mice, i.e., injected with neither TC1-nLuc cells, nor lentiviral vectors, yet injected with Z108.

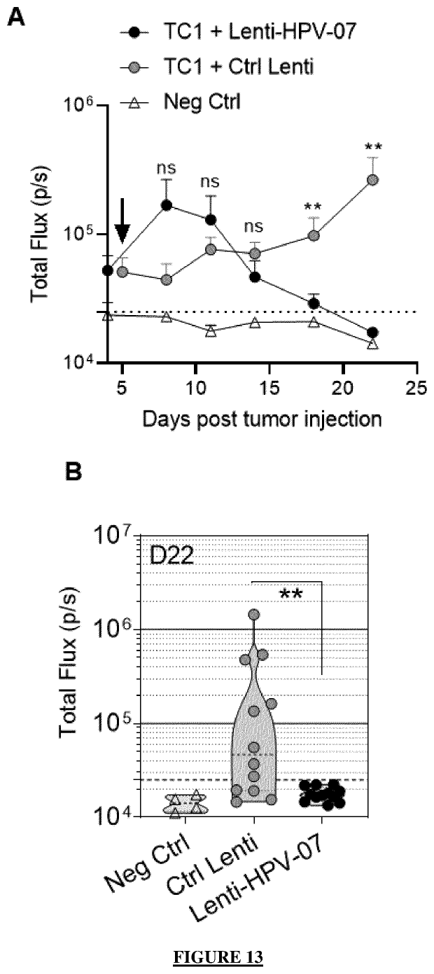

The results obtained are represented in FIGS. 13A and 13B.

The inventors had firmly established that a single intramuscular injection of Lenti-HPV-07 vaccine (I-5759) according to the invention completely eradicates, in 100% of animals, subcutaneously engrafted TC1 tumors. However, in human, many cancers, including HPV-induced cancers, are located in mucosal sites.

Therefore, the present experiment evaluated the capacity of Lenti-HPV-07 to inhibit tumor growth in a mucosal site.

To address this question, a TC1 cell line expressing stably the nanoluciferase reporter gene (TC1-nLuc) was developed. After TC1-nLuc intravenous injection, mice readily developed lung metastatic foci.

Longitudinal tumor outgrowth was followed on live animals by bioluminescence imaging.

Five days after tumor injection, mice received a single intramuscular injection of Lenti-HPV-07 (1.109 TU/mouse) or a Control Lenti (empty vector) (FIG. 13A).

All mice having received the Lenti-HPV-07 were cured at day 22 post tumor injection, whereas pulmonary metastatic foci continued to grow in the control group. The difference observed between the average of the bioluminescence signal in the two groups was largely statistically significant (FIGS. 13 A and B).

This observation indicates in a clear-cut manner that a vaccine according to the invention is able to eradicate lung tumors, as efficiently as subcutaneously established tumors.

SEQUENCES

SEQ ID NO: 1 is a nucleic acid sequence encoding the E6 protein from HPV 16
(NC_001526.4)
ATGCACCAAAAGAGAACTGCAATGTTTCAGGACCCACAGGAGCGACCCAGAAAGTTACC
ACAGTTATGCACAGAGCTGCAAACAACTATACATGATATAATATTAGAATGTGTGTACTG
CAAGCAACAGTTACTGCGACGTGAGGTATATGACTTTGCTTTTCGGGATTTATGCATAGT
ATATAGAGATGGGAATCCATATGCTGTATGTGATAAATGTTTAAAGTTTTATTCTAAAATT
AGTGAGTATAGACATTATTGTTATAGTTTGTATGGAACAACATTAGAACAGCAATACAAC
AAACCGTTGTGTGATTTGTTAATTAGGTGTATTAACTGTCAAAAGCCACTGTGTCCTGAAG
AAAAGCAAAGACATCTGGACAAAAAGCAAAGATTCCATAATATAAGGGGTCGGTGGACC
GGTCGATGTATGTCTTGTTGCAGATCATCAAGAACACGTAGAGAAACCCAGCTGTAA
SEQ ID NO: 2 is the nucleic acid sequence encoding a non-oncogenic variant of the E6
protein from HPV 16
GACCCCCAAGAACGGCCCAGAAAGCTGCCCCAGCTGTGCACCGAGCTGCAGACCACCAT
CCACGACATCATCCTGGAATGCGTGTACTGCAAGCAGCAGCTGCTGAGAAGAGAGGTGT
ACGACTTCGCCTTCCGGGACCTGTGCATCGTGTACCGGAACCCCTACGCCGTGTGCGACA
AGTGCCTGAAGTTCTACAGCAAGATCAGCGAGTACCGGCACTACTGCTACAGCCTGTACG
GCACCACCCTGGAACAGCAGTACAACAAGCCCCTGTGCGACCTGCTGATCAGATGCATCA
ACTGCCAGAAGCCCCTGCGGTTCCACAACATCCGGGGCAGATGGACCGGCCGGTGCATG
AGCTGCTGCAGA
SEQ ID NO: 3 is the nucleic acid sequence encoding a non-oncogenic variant of the E6
protein from HPV 16
ATGGGCACCCTGGGCATCGTGTGCCCCATCGACCCCCAAGAACGGCCCAGAAAGCTGCCC
CAGCTGTGCACCGAGCTGCAGACCACCATCCACGACATCATCCTGGAATGCGTGTACTGC
AAGCAGCAGCTGCTGAGAAGAGAGGTGTACGACTTCGCCTTCCGGGACCTGTGCATCGTG
TACCGGAACCCCTACGCCGTGTGCGACAAGTGCCTGAAGTTCTACAGCAAGATCAGCGAG
TACCGGCACTACTGCTACAGCCTGTACGGCACCACCCTGGAACAGCAGTACAACAAGCCC
CTGTGCGACCTGCTGATCAGATGCATCAACTGCCAGAAGCCCCTGCGGTTCCACAACATC
CGGGGCAGATGGACCGGCCGGTGCATGAGCTGCTGCAGA
SEQ ID NO: 4 is the nucleic acid sequence encoding a non-oncogenic variant of the E6
protein from HPV 16
ATGGACCCCCAAGAACGGCCCAGAAAGCTGCCCCAGCTGTGCACCGAGCTGCAGACCAC
CATCCACGACATCATCCTGGAATGCGTGTACTGCAAGCAGCAGCTGCTGAGAAGAGAGG
TGTACGACTTCGCCTTCCGGGACCTGTGCATCGTGTACCGGAACCCCTACGCCGTGTGCG
ACAAGTGCCTGAAGTTCTACAGCAAGATCAGCGAGTACCGGCACTACTGCTACAGCCTGT
ACGGCACCACCCTGGAACAGCAGTACAACAAGCCCCTGTGCGACCTGCTGATCAGATGC
ATCAACTGCCAGAAGCCCCTGCGGTTCCACAACATCCGGGGCAGATGGACCGGCCGGTG
CATGAGCTGCTGCAGA
SEQ ID NO: 5 is the nucleic acid sequence encoding a non-oncogenic variant of the E6
protein from HPV 16
TTTCAGGACCCCCAGGAAAGGCCCAGGAAGTTGCCCCAGCTCTGCACCGAACTGCAGACC
ACCATTCATGACATCATCCTCGAATGCGTGTACTGCAAGCAGCAGCTCCTGAGGAGGGAG
GTGTACGATTTCGCCTTCAGAGACGGCTGTATCGTCTACAGGAACCCCTATGCCGTCTGC
GACAAATGCCTGAAGTTTTATTCCAAGATCTCCGAGTACAGGCACTATTGCTACAGCCTG
TATGGGACCACCCTGGAGCAGCAGTACAACAAGCCCCTGTGCGACCTCCTGATCAGGTGC
ATCAACTGCCAGAAGCCCCTGAGGTTCCACAACATCCGCGGCAGGTGGACCGGAAGGTG
CATGTCCTGCTGCAGG
SEQ ID NO: 6 is the nucleic acid sequence encoding a non-oncogenic variant of the E6
protein from HPV 16
TTCCAGGACCCCCAGGAGAGGCCCAGGAAACTGCCCCAGTTGTGCACCGAGCTCCAGAC
AACCATCCACGACATCATCCTGGAGTGCGTGTACTGTAAGCAGCAGTTGCTGAGGAGAGA
GGTGTATGACTTCGCCTTCAGAGACGGATGCATTGTCTATAGGAACCCCTACGCCGTGTG
CGACAAGTGCCTGAAGTTCTACTCCAAGATCAGTGAGTACAGGCATTACTGCTACAGCCT
GTATGGAACCACACTGGAACAGCAGTACAACAAGCCCCTGTGCGACCTCCTGATTAGGTG
CATCAACTGCCAGAAGCCCCTCAGGTTCCACAACATCCGGGGCAGGTGGACCGGAAGGT
GCATGTCCTGCTGCAGGTCC
SEQ ID NO: 7 is the amino acid sequence of the Wild Type (WT) E6 protein from HPV 16
MHQKRTAMFQDPQERPRKLPQLCTELQTTIHDIILECVYCKQQLLRREVYDFAFRDLCIVYRD
GNPYAVCDKCLKFYSKISEYRHYCYSLYGTTLEQQYNKPLCDLLIRCINCQKPLCPEEKQRHL
DKKQRFHNIRGRWTGRCMSCCRSSRTRRETQL
SEQ ID NO: 8 is the amino acid sequence of a non-oncogenic variant of the E6 protein from
HPV 16
DPQERPRKLPQLCTELQTTIHDIILECVYCKQQLLRREVYDFAFRDLCIVYRNPYAVCDKCLKF
YSKISEYRHYCYSLYGTTLEQQYNKPLCDLLIRCINCQKPLRFHNIRGRWTGRCMSCCR
SEQ ID NO: 9 is the amino acid sequence of a non-oncogenic variant of the E6 protein from
HPV 16
MGTLGIVCPIDPQERPRKLPQLCTELQTTIHDIILECVYCKQQLLRREVYDFAFRDLCIVYRNPY
AVCDKCLKFYSKISEYRHYCYSLYGTTLEQQYNKPLCDLLIRCINCQKPLRFHNIRGRWTGRC
MSCCR
SEQ ID NO: 10 is the amino acid sequence of a non-oncogenic variant of the E6 protein
from HPV 16
MDPQERPRKLPQLCTELQTTIHDIILECVYCKQQLLRREVYDFAFRDLCIVYRNPYAVCDKCL
KFYSKISEYRHYCYSLYGTTLEQQYNKPLCDLLIRCINCQKPLRFHNIRGRWTGRCMSCCR
SEQ ID NO: 11 is the amino acid sequence of a non-oncogenic variant of the E6 protein
from HPV 16
FQDPQERPRKLPQLCTELQTTIHDIILECVYCKQQLLRREVYDFAFRDGCIVYRNPYAVCDKC
LKFYSKISEYRHYCYSLYGTTLEQQYNKPLCDLLIRCINCQKPLRFHNIRGRWTGRCMSCCR
SEQ ID NO: 12 is the amino acid sequence of a non-oncogenic variant of the E6 protein
from HPV 16
FQDPQERPRKLPQLCTELQTTIHDIILECVYCKQQLLRREVYDFAFRDGCIVYRNPYAVCDKC
LKFYSKISEYRHYCYSLYGTTLEQQYNKPLCDLLIRCINCQKPLRFHNIRGRWTGRCMSCCRS
SEQ ID NO: 13 is the nucleic acid sequence encoding the E7 protein from HPV 16 (NP-
041326.1)
ATGCATGGAGATACACCTACATTGCATGAATATATGTTAGATTTGCAACCAGAGACAACT
GATCTCTACTGTTATGAGCAATTAAATGACAGCTCAGAGGAGGAGGATGAAATAGATGG
TCCAGCTGGACAAGCAGAACCGGACAGAGCCCATTACAATATTGTAACCTTTTGTTGCAA
GTGTGACTCTACGCTTCGGTTGTGCGTACAAAGCACACACGTAGACATTCGTACTTTGGA
AGACCTGTTAATGGGCACACTAGGAATTGTGTGCCCCATCTGTTCTCAGAAACCATAA
SEQ ID NO: 14 is a nucleic acid sequence encoding a non-oncogenic variant of the E7
protein from HPV 16
ACCCCCACCCTGCACGAGTACATGCTGGACCTGCAGCCCGAGACAACCGACCCCGACCG
GGCCCACTACAATATCGTGACCTTCTGCTGCAAGTGCGACAGCACCCTGCGGCTGTGCGT
GCAGAGCACCCACGTGGACATCCGGACCCTGGAAGATCTGCTGATGGGCACCCTGGGCA
TCGTGTGCCCCATT
SEQ ID NO: 15 is a nucleic acid sequence encoding a non-oncogenic variant of the E7
protein from HPV 16
CCCGGAGACACCCCCACCCTGCACGAATACATGCTGGACCTGCAGCCCGAAACCACCGA
CCCCGACCGCGCTCACTACAACATCGTTACATTCTGTTGTAAATGCGACTCCACCCTGAG
AAGATGCGTGCAGTCCACCCACGTGGACATCAGGACCCTGGAGGACCTCCTCATGGGAA
CCCTGGGTATCGTCTGCCCCATC
SEQ ID NO: 16 is the amino acid sequence of the Wild Type (WT) E7 protein from HPV 16
MHGDTPTLHEYMLDLQPETTDLYCYEQLNDSSEEEDEIDGPAGQAEPDRAHYNIVTFCCKCD
STLRLCVQSTHVDIRTLEDLLMGTLGIVCPICSQKP
SEQ ID NO: 17 is the amino acid sequence of a non-oncogenic variant of the E7 protein
from HPV 16
TPTLHEYMLDLQPETTDPDRAHYNIVTFCCKCDSTLRLCVQSTHVDIRTLEDLLMGTLGIVCPI
SEQ ID NO: 18 is the amino acid sequence of a non-oncogenic variant of the E7 protein
from HPV 16
PGDTPTLHEYMLDLQPETTDPDRAHYNIVTFCCKCDSTLRRCVQSTHVDIRTLEDLLMGTLGI
VCPI
SEQ ID NO: 19 is the nucleic acid sequence encoding the E6 protein from HPV 18
(MF288727.1)
ATGGCGCGCTTTGAGGATCCAACACGGCGACCCTACAAGCTACCTGATCTGTGCACGGAA
CTGAACACTTCACTGCAAGACATAGAAATAACCTGTGTATATTGCAAGACAGTATTGGAA
CTTACAGAGGTATTTGAATTTGCATTTAAAGATTTATTTGTGGTGTATAGAGACAGTATAC
CGCATGCTGCATGCCATAAATGTATAGATTTTTATTCTAGAATTAGAGAATTAAGACATT
ATTCAGACTCTGTGTATGGAGACACATTGGAGAAACTAACTAACACTGGGTTATACAATT
TATTAATAAGGTGCCTGCGGTGCCAGAAACCGTTGAATCCAGCAGAAAAACTTAGACACC
TTAATGAAAAACGACGATTCCACAACATAGCTGGGCACTATAGAGGCCAGTGCCATTCGT
GCTGCAACCGAGCACGACAGGAAAGACTCCAACGACGCAGAGAAACACAAGTATAA
SEQ ID NO: 20 is the nucleic acid sequence encoding a non-oncogenic variant of the E6
protein from HPV 18
CCCTACAAGCTGCCTGACCTGTGTACAGAGCTGAACACCTCCCTGCAGGACATCGAGATC
ACCTGTGTGTATTGCAAGACCGTGCTGGAACTGACCGAGGTGTTCGAGTTTGCCTTCAAG
GATCTGTTCGTGGTGTACCGGGACAGCATCCCCCACGCCGCCTGCCACAAGCTGGAAAAG
CTGACCAACACCGGCCTGTACAACCTGCTGATTCGGTGCCTGCGGTGTCAGAAGCCTCTG
AACCCCGCCGAGAAGCTGCGGCACCTGAACGAGAAGCGGAGATTCCACAATATCGCC
SEQ ID NO: 21 is the nucleic acid sequence encoding a non-oncogenic variant of the E6
protein from HPV 18
CCCTACAAGCTGCCTGACCTGTGTACAGAGCTGAACACCTCCCTGCAGGACATCGAGATC
ACCTGTGTGTATTGCAAGACCGTGCTGGAACTGACCGAGGTGTTCGAGTTTGCCTTCAAG
GATCTGTTCGTGGTGTACCGGGACAGCATCCCCCACGCCGCCTGCCACAAG
SEQ ID NO: 22 is the nucleic acid sequence encoding a non-oncogenic variant of the E6
protein from HPV 18
CCCTACAAGCTGCCCGATCTGTGCACAGAGCTGAACACCTCCCTGCAGGACATCGAGATC
ACCTGCGTCTACTGCAAGACCGTGCTGGAACTGACCGAGGTGTTCGAATTCGCCTTCAAG
GACGGCTTCGTGGTGTACAGGGACAGCATTCCCCACGCCGCCTGCCATAAGCTGGAGAAA
CTGACCAACACCGGACTGTATAACCTGCTGATCAGGTGTCTGAGGTGCCAGAAGGCAGA
GAAACTGAGACATCTGAACGAGAAAAGGAGGTTCCACAATATTGCCGGGCACTGATAA
SEQ ID NO: 23 is the nucleic acid sequence encoding a non-oncogenic variant of the E6
protein from HPV 18
ATGAGGCGGCCCTACAAGCTGCCCGACCTGTGCACCGAGCTGAACACCTCCCTGCAGGAC
ATCGAGATCACCTGCGTGTACTGCAAGACCGTGCTGGAGCTGACCGAGGTGTTCGAATTC
GCATTCAAGGACGGATTCGTCGTGTATAGGGACAGCATTCCACACGCCGCCTGCCACAAG
CTGGAGAAATTGACTAACACCGGACTGTATAATCTGCTGATCCGGTGCCTGAGGTGTCAG
AAGGCCGAGAAGCTGAGGCATCTGAACGAGAAAAGGAGATTCCACAATATCGCCGGACA
C
SEQ ID NO: 24 is the amino acid sequence of the Wild Type (WT) E6 protein from HPV 18
MARFEDPTRRPYKLPDLCTELNTSLQDIEITCVYCKTVLELTEVFEFAFKDLFVVYRDSIPHAA
CHKCIDFYSRIRELRHYSDSVYGDTLEKLINTGLYNLLIRCLRCQKPLNPAEKLRHLNEKRRF
HNIAGHYRGQCHSCCNRARQERLQRRRETQV
SEQ ID NO: 25 is the amino acid sequence of a non-oncogenic variant of the E6 protein
from HPV 18
PYKLPDLCTELNTSLQDIEITCVYCKTVLELTEVFEFAFKDLFVVYRDSIPHAACHKLEKLTNT
GLYNLLIRCLRCQKPLNPAEKLRHLNEKRRFHNIA
SEQ ID NO: 26 is the amino acid sequence of a non-oncogenic variant of the E6 protein
from HPV 18
PYKLPDLCTELNTSLQDIEITCVYCKTVLELTEVFEFAFKDLFVVYRDSIPHAACHK
SEQ ID NO: 27 is the amino acid sequence of a non-oncogenic variant of the E6 protein
from HPV 18
PYKLPDLCTELNTSLQDIEITCVYCKTVLELTEVFEFAFKDGFVVYRDSIPHAACHKLEKLTNT
GLYNLLIRCLRCQKAEKLRHLNEKRRFHNIAGH
SEQ ID NO: 28 is the amino acid sequence of a non-oncogenic variant of the E6 protein
from HPV 18
MRRPYKLPDLCTELNTSLQDIEITCVYCKTVLELTEVFEFAFKDGFVVYRDSIPHAACHKLEKL
TNTGLYNLLIRCLRCQKAEKLRHLNEKRRFHNIAGH
SEQ ID NO: 29 is the nucleic acid sequence encoding the E7 protein from HPV 18
(NC_001357.1)
ATGCATGGACCTAAGGCAACATTGCAAGACATTGTATTGCATTTAGAGCCCCAAAATGAA
ATTCCGGTTGACCTTCTATGTCACGAGCAATTAAGCGACTCAGAGGAAGAAAACGATGAA
ATAGATGGAGTTAATCATCAACATTTACCAGCCCGACGAGCCGAACCACAACGTCACACA
ATGTTGTGTATGTGTTGTAAGTGTGAAGCCAGAATTGAGCTAGTAGTAGAAAGCTCAGCA
GACGACCTTCGAGCATTCCAGCAGCTGTTTCTGAACACCCTGTCCTTTGTGTGTCCGTGGT
GTGCATCCCAGCAGTAA
SEQ ID NO: 30 is the nucleic acid sequence encoding a non-oncogenic variant of the E7
protein from HPV 18
AAGGCCACACTGCAGGATATCGTGCTGCACCTGGAACCCCAGAACGAGATCCCCGTGGA
CAGCGAGGAAGAGAACGACGAGATCGACGGCGTGAACCACCAGCATCTGCCCGCCAGAA
GGGCCGAGCCCCAGAGACACACCATGCTGTGCATGTGTTGCAAATGCGAGGCCCGGATC
AAGCTGGTGGTGGAAAGCAGCGCCGACGACCTGCGGGCCTTCCAGCAGCTGTTCCTGAAC
ACCCTGTCCTTCGTGTGCCCTTGG
SEQ ID NO: 31 is the nucleic acid sequence encoding a non-oncogenic variant of the E7
protein from HPV 18
GGACCTAAAGCCACCCTCCAGGACATCGTGCTGCACCTGGAGCCCCAGAACGAGATCCCC
GTCGACTCAGAGGAGGAGAACGACGAAATTGACGGCGTCAACCACCAGCACCTGCCCGC
TCGCAGAGCCGAACCCCAGAGACACACCATGCTCTGCATGTGCTGCAAATGCGAGGCCC
GGATTAAGCTGGTGGTGGAGAGCTCCGCCGACGATCTGAGAGCCTTCCAGCAGCTCTTCC
TGAACACCCTGTCCTTCGTGTGCCCCTGG
SEQ ID NO: 32 is the nucleic acid sequence encoding a non-oncogenic variant of the E7
protein from HPV 18
GGACCTAAAGCCACCCTCCAGGACATCCGTCTGGAGCCCCAGAACGAGATCCCCGTCGAC
TCAGAGGAGGAGAACGACGAAATTGACGGCAACCACCAGCACCTGCCCGCTCGCAGAGC
CGAACCCCAGAGACACACCATGCTCTGCATGTGCTGCAAATGCGAGGCCCGGATTAAGCT
GGTGGTGGAGAGCTCCGCCGACGATCTGAGAGCCTTCCAGCAGCTCTTCCTGGATTCCTT
CGTGTGCCCCTGG
SEQ ID NO: 33 is the amino acid sequence of the Wild Type (WT) E7 protein from HPV 18
MHGPKATLQDIVLHLEPQNEIPVDLLCHEQLSDSEEENDEIDGVNHQHLPARRAEPQRHTMLC
MCCKCEARIELVVESSADDLRAFQQLFLNTLSFVCPWCASQQ
SEQ ID NO: 34 is the amino acid sequence of a non-oncogenic variant of the E7 protein
from HPV 18
KATLQDIVLHLEPQNEIPVDSEEENDEIDGVNHQHLPARRAEPQRHTMLCMCCKCEARIKLVV
ESSADDLRAFQQLFLNTLSFVCPW
SEQ ID NO: 35 is the amino acid sequence of a non-oncogenic variant of the E7 protein
from HPV 18
GPKATLQDIVLHLEPQNEIPVDSEEENDEIDGVNHQHLPARRAEPQRHTMLCMCCKCEARIKL
VVESSADDLRAFQQLFLNTLSFVCPW
SEQ ID NO: 36 is the amino acid sequence of a non-oncogenic variant of the E7 protein
from HPV 18
GPKATLQDIRLEPQNEIPVDSEEENDEIDGNHQHLPARRAEPQRHTMLCMCCKCEARIKLVVE
SSADDLRAFQQLFLDSFVCPW
SEQ ID NO: 37 is the nucleic acid sequence encoding the cPPT/CTS sequence
AATTTTAAAAGAAAAGGGGGGATTGGGGGGTACAGTGCAGGGGAAAGAATAGTAGACAT
AATAGCAACAGACATACAAACTAAAGAATTACAAAAACAAATTACAAAAATTCAAAATT
TT
SEQ ID NO: 38 is the nucleic acid sequence encoding a mutant form of the woodchuck
hepatitis B virus (WHV) post-transcriptional regulatory element (WPRE)
TTCCCGATAATCAACCTCTGGATTACAAAATTTGTGAAAGATTGACTGGTATTCTTAACTA
TGTTGCTCCTTTTACGCTATGTGGATACGCTGCTTTAATGCCTTTGTATCATGCTATTGCTT
CCCGTATGGCTTTCATTTTCTCCTCCTTGTATAAATCCTGGTTGCTGTCTCTTTATGAGGAG
TTGTGGCCCGTTGTCAGGCAACGTGGCGTGGTGTGCACTGTGTTTGCTGACGCAACCCCC
ACTGGTTGGGGCATTGCCACCACCTGTCAGCTCCTTTCCGGGACTTTCGCTTTCCCCCTCC
CTATTGCCACGGCGGAACTCATCGCCGCCTGCCTTGCCCGCTGCTGGACAGGGGCTCGGC
TGTTGGGCACTGACAATTCCGTGGTGTTGTCGGGGAAGCTGACGTCCTTTCCGCGGCTGCT
CGCCTGTGTTGCCACCTGGATTCTGCGCGGGACGTCCTTCTGCTACGTCCCTTCGGCCCTC
AATCCAGCGGACCTTCCTTCCCGCGGCCTGCTGCCGGCTCTGCGGCCTCTTCCGCGTCTTC
GCCTTCGCCCTCAGACGAGTCGGATCTCCCTTTGGGCCGCCTCCCCGC
SEQ ID NO: 39 is a synthetic E7HPV16-derived peptide containing the RAHYNIVTF H-2D^{b}_
restricted T-cell epitope
ETTDPDRAHYNIVTF
SEQ ID NO: 40 is a synthetic E7HPV16-derived peptide containing the RAHYNIVTF H-2D^{b}_
restricted T-cell epitope
PDRAHYNIVTFCCKC
SEQ ID NO: 41 is the nucleic acid sequence encoding an antigen construct of
the lentiviral vector filed at the CNCM under accession number I-5759
ATGCCCGGAGACACCCCCACCCTGCACGAATACATGCTGGACCTGCAGCCCGAA
ACCACCGACCCCGACCGCGCTCACTACAACATCGTTACATTCTGTTGTAAATGCG
ACTCCACCCTGAGAAGATGCGTGCAGTCCACCCACGTGGACATCAGGACCCTGGA
GGACCTCCTCATGGGAACCCTGGGTATCGTCTGCCCCATCGCCTCCCAGGCTTTTC
AGGACCCCCAGGAAAGGCCCAGGAAGTTGCCCCAGCTCTGCACCGAACTGCAGA
CCACCATTCATGACATCATCCTCGAATGCGTGTACTGCAAGCAGCAGCTCCTGAG
GAGGGAGGTGTACGATTTCGCCTTCAGAGACGGCTGTATCGTCTACAGGAACCCC
TATGCCGTCTGCGACAAATGCCTGAAGTTTTATTCCAAGATCTCCGAGTACAGGC
ACTATTGCTACAGCCTGTATGGGACCACCCTGGAGCAGCAGTACAACAAGCCCCT
GTGCGACCTCCTGATCAGGTGCATCAACTGCCAGAAGCCCCTGAGGTTCCACAAC
ATCCGCGGCAGGTGGACCGGAAGGTGCATGTCCTGCTGCAGGTCCGCCGGCCCCG
GACCTAAAGCCACCCTCCAGGACATCGTTCTCCACCTGGAGCCCCAGAACGAGAT
CCCCGTGGACTCAGAAGAGGAGAACGACGAGATCGACGGCGTCAACCACCAGCA
CCTGCCCGCTCGCAGAGCCGAACCCCAGAGACACACCATGCTCTGCATGTGCTGC
AAATGCGAAGCCCGGATTAAGTTGGTGGTGGAAAGCAGCGCCGACGATCTGAGG
GCCTTCCAGCAGCTCTTCCTCAACACCCTGTCCTTCGTGTGCCCCTGGGTGGGCGA
GCCCGGTAGAACCATCCCCTACAAGCTGCCCGATCTGTGCACAGAGCTGAACACC
TCCCTGCAGGACATCGAGATCACCTGCGTCTACTGCAAGACCGTGCTGGAACTGA
CCGAGGTGTTCGAATTCGCCTTCAAGGACGGCTTCGTGGTGTACAGGGACAGCAT
TCCCCACGCCGCCTGCCATAAGCTGGAGAAACTGACCAACACCGGACTGTATAAC
CTGCTGATCAGGTGTCTGAGGTGCCAGAAGGCAGAGAAACTGAGACATCTGAAC
GAGAAAAGGAGGTTCCACAATATTGCCGGGCACTGATAA
SEQ ID NO: 42 is the amino acid sequence of the antigen construct encoded by
the lentiviral vector filed at the CNCM under accession number I-5759
MPGDTPTLHEYMLDLQPETTDPDRAHYNIVTFCCKCDSTLRRCVQSTHVDIRTLEDL
LMGTLGIVCPIASQAFQDPQERPRKLPQLCTELQTTIHDIILECVYCKQQLLRREVYDF
AFRDGCIVYRNPYAVCDKCLKFYSKISEYRHYCYSLYGTTLEQQYNKPLCDLLIRCIN
CQKPLRFHNIRGRWTGRCMSCCRSAGPGPKATLQDIVLHLEPQNEIPVDSEEENDEID
GVNHQHLPARRAEPQRHTMLCMCCKCEARIKLVVESSADDLRAFQQLFLNTLSFVCP
WVGEPGRTIPYKLPDLCTELNTSLQDIEITCVYCKTVLELTEVFEFAFKDGFVVYRDSI
PHAACHKLEKLTNTGLYNLLIRCLRCQKAEKLRHLNEKRRFHNIAGH
SEQ ID NO: 43 is the nucleic acid sequence encoding an antigen construct of
the lentiviral vector filed at the CNCM under accession number I-5760
ATGTTCCAGGACCCCCAGGAGAGGCCCCGGAAGTTGCCCCAGCTGTGCACCGAG
CTGCAGACCACCATCCACGACATCATCCTCGAATGCGTGTACTGCAAGCAGCAGC
TGCTGAGGAGGGAGGTGTATGACTTTGCCTTCAGAGACGGATGCATTGTCTACAG
GAACCCCTACGCCGTGTGCGACAAATGCCTGAAGTTCTACTCCAAGATCAGCGAG
TACAGGCACTACTGCTACTCCCTGTACGGCACCACCCTCGAACAGCAGTACAACA
AACCCCTGTGCGACCTCCTGATTAGGTGCATCAACTGCCAGAAGCCCCTCAGGTT
CCACAACATCCGCGGCCGCTGGACCGGCCGATGCATGTCTTGCTGCAGGGGCCCC
GACGACCCCTACAAGCTCCCCGACCTGTGCACCGAACTCAACACCTCCCTGCAGG
ACATCGAGATCACCTGCGTGTATTGCAAGACCGTGCTGGAGCTGACCGAGGTTTT
CGAATTTGCCTTTAAGGACGGCTTCGTCGTGTATAGGGACTCCATCCCCCACGCC
GCCTGCCATAAGCTGGAGAAGCTCACCAACACCGGACTGTATAATCTGCTGATCA
GGTGCCTCAGGTGCCAGAAGGCAGAAAAGCTGAGGCATCTCAACGAGAAGCGCC
GGTTCCACAATATTGCCGGCCCCGGAGACACCCCCACACTCCATGAGTACATGCT
CGACCTGCAGCCCGAAACCACCGACCCCGACAGAGCCCACTACAACATCGTGAC
CTTCTGCTGCAAGTGCGACTCCACCCTGAGAAGATGCGTGCAGTCCACCCACGTG
GACATCCGCACACTCGAAGACCTGCTGATGGGAACCCTGGGCATCGTGTGCCCCA
TCGGCCCCGATGACAAGGCCACCTTGCAGGACATCGTGCTGCACCTGGAACCACA
GAACGAGATCCCCGTCGACTCCGAAGAAGAAAACGACGAAATCGACGGAGTGAA
TCACCAGCACCTGCCCGCCAGAAGGGCCGAGCCTCAGAGACACACCATGCTCTGC
ATGTGCTGCAAATGCGAAGCCAGGATTAAGCTGGTGGTGGAGAGCAGCGCCGAC
GACCTGAGGGCCTTCCAGCAGCTCTTCCTGAACACACTGTCCTTCGTGTGCCCCTG
GGCCTGATAA
SEQ ID NO: 44 is the amino acid sequence of the antigen construct encoded
by the lentiviral vector filed at the CNCM under accession number I-5760
MFQDPQERPRKLPQLCTELQTTIHDIILECVYCKQQLLRREVYDFAFRDGCIVYRNPY
AVCDKCLKFYSKISEYRHYCYSLYGTTLEQQYNKPLCDLLIRCINCQKPLRFHNIRGR
WTGRCMSCCRGPDDPYKLPDLCTELNTSLQDIEITCVYCKTVLELTEVFEFAFKDGFV
VYRDSIPHAACHKLEKLTNTGLYNLLIRCLRCQKAEKLRHLNEKRRFHNIAGPGDTPT
LHEYMLDLQPETTDPDRAHYNIVTFCCKCDSTLRRCVQSTHVDIRTLEDLLMGTLGIV
CPIGPDDKATLQDIVLHLEPQNEIPVDSEEENDEIDGVNHQHLPARRAEPQRHTMLCM
CCKCEARIKLVVESSADDLRAFQQLFLNTLSFVCPWA
SEQ ID NO: 45 is the nucleic acid sequence encoding an antigen construct
of the lentiviral vector filed at the CNCM under accession number I-5761
ATGAGGCGGCCCTACAAGCTGCCCGACCTGTGCACCGAGCTGAACACCTCCCTGC
AGGACATCGAGATCACCTGCGTGTACTGCAAGACCGTGCTGGAGCTGACCGAGG
TGTTCGAATTCGCATTCAAGGACGGATTCGTCGTGTATAGGGACAGCATTCCACA
CGCCGCCTGCCACAAGCTGGAGAAATTGACTAACACCGGACTGTATAATCTGCTG
ATCCGGTGCCTGAGGTGTCAGAAGGCCGAGAAGCTGAGGCATCTGAACGAGAAA
AGGAGATTCCACAATATCGCCGGACACTTCCAGGACCCCCAGGAGAGGCCCAGG
AAACTGCCCCAGTTGTGCACCGAGCTCCAGACAACCATCCACGACATCATCCTGG
AGTGCGTGTACTGTAAGCAGCAGTTGCTGAGGAGAGAGGTGTATGACTTCGCCTT
CAGAGACGGATGCATTGTCTATAGGAACCCCTACGCCGTGTGCGACAAGTGCCTG
AAGTTCTACTCCAAGATCAGTGAGTACAGGCATTACTGCTACAGCCTGTATGGAA
CCACACTGGAACAGCAGTACAACAAGCCCCTGTGCGACCTCCTGATTAGGTGCAT
CAACTGCCAGAAGCCCCTCAGGTTCCACAACATCCGGGGCAGGTGGACCGGAAG
GTGCATGTCCTGCTGCAGGTCCGCCGGCCCCGGACCTAAAGCCACCCTCCAGGAC
ATCGTGCTGCACCTGGAGCCCCAGAACGAGATCCCCGTCGACTCAGAGGAGGAG
AACGACGAAATTGACGGCGTCAACCACCAGCACCTGCCCGCTCGCAGAGCCGAA
CCCCAGAGACACACCATGCTCTGCATGTGCTGCAAATGCGAGGCCCGGATTAAGC
TGGTGGTGGAGAGCTCCGCCGACGATCTGAGAGCCTTCCAGCAGCTCTTCCTGAA
CACCCTGTCCTTCGTGTGCCCCTGGGCCGGTCCCGGTGACACACCTACCCTGCAC
GAGTACATGCTCGATCTGCAGCCCGAGACCACCGACCCCGATCGCGCACACTACA
ACATCGTGACCTTCTGCTGCAAATGTGACAGCACCCTGAGACGGTGCGTCCAGTC
CACCCACGTTGACATCCGCACCCTCGAAGACCTGCTCATGGGAACCCTGGGCATC
GTGTGCCCCATCGCCTGATAA
SEQ ID NO: 46 is the amino acid sequence of the antigen construct encoded
by the lentiviral vector filed at the CNCM under accession number I-5761
MRRPYKLPDLCTELNTSLQDIEITCVYCKTVLELTEVFEFAFKDGFVVYRDSIPHAAC
HKLEKLTNTGLYNLLIRCLRCQKAEKLRHLNEKRRFHNIAGHFQDPQERPRKLPQLC
TELQTTIHDIILECVYCKQQLLRREVYDFAFRDGCIVYRNPYAVCDKCLKFYSKISEYR
HYCYSLYGTTLEQQYNKPLCDLLIRCINCQKPLRFHNIRGRWTGRCMSCCRSAGPGP
KATLQDIVLHLEPQNEIPVDSEEENDEIDGVNHQHLPARRAEPQRHTMLCMCCKCEA
RIKLVVESSADDLRAFQQLFLNTLSFVCPWAGPGDTPTLHEYMLDLQPETTDPDRAH
YNIVTFCCKCDSTLRRCVQSTHVDIRTLEDLLMGTLGIVCPIA
SEQ ID NO: 47 is the nucleic acid sequence encoding an antigen construct of
the lentiviral vector filed at the CNCM under accession number I-5762
ATGGGCCCTAAGGCCACCCTGCAGGACATCGTGCTGCACTTGGAGCCCCAGAACG
AGATCCCCGTGGACAGCGAGGAGGAGAACGACGAAATCGACGGCGTGAACCACC
AGCACCTGCCCGCAAGAAGGGCCGAACCCCAGAGGCACACCATGCTCTGCATGT
GCTGCAAATGCGAGGCCAGGATCAAGCTGGTGGTGGAAAGCAGCGCCGACGATC
TGAGGGCATTCCAGCAGCTGTTCCTGAACACCCTCTCCTTCGTGTGCCCTGGGGA
ACCCGGCAGGACCATCCCCTATAAACTGCCCGACCTCTGCACCGAGCTGAACACC
TCCCTGCAGGACATTGAGATCACCTGCGTCTACTGCAAAACCGTCCTGGAACTGA
CCGAGGTGTTCGAGTTCGCCTTCAAAGACGGCTTCGTCGTGTACAGGGACAGCAT
CCCCCACGCCGCCTGCCATAAGCTGGAGAAACTGACCAACACCGGCCTGTACAAC
CTGCTGATCCGGTGCCTGAGATGTCAGAAGGCCGAGAAACTGAGGCACCTCAAC
GAGAAAAGGAGATTCCACAATATTGCCGGGCCCGGCGACACCCCAACCCTGCAC
GAATACATGCTCGACCTGCAGCCCGAAACCACCGACCCCGACAGAGCCCACTAC
AACATCGTGACCTTCTGCTGCAAGTGCGACTCCACCCTGAGAAGATGCGTGCAGT
CCACCCACGTGGACATCCGCACACTCGAAGACCTGCTGATGGGAACCCTGGGCAT
CGTGTGCCCCATCGCTTCCCAGGCCTTTCAGGACCCCCAGGAACGGCCAAGAAAG
CTGCCCCAGCTCTGCACCGAACTGCAGACCACCATCCACGACATCATCCTGGAAT
GCGTCTACTGTAAGCAGCAGTTGCTGAGGAGGGAGGTGTATGATTTCGCCTTCAG
AGACGGCTGCATCGTCTACAGGAACCCCTACGCCGTGTGCGACAAATGCCTGAAG
TTCTACTCCAAGATCTCCGAATACAGACACTATTGCTACAGCCTGTACGGCACCA
CCCTCGAACAGCAGTACAACAAACCCCTGTGCGACCTCCTGATCAGGTGCATCAA
CTGCCAGAAGCCCCTCCGGTTCCACAACATCCGAGGAAGATGGACCGGCCGGTG
CATGTCCTGCTGCAGGTCCTGATAA
SEQ ID NO: 48 is the amino acid sequence of the antigen construct
encoded by the lentiviral
vector filed at the CNCM under accession number I-5762
MGPKATLQDIVLHLEPQNEIPVDSEEENDEIDGVNHQHLPARRAEPQRHTMLCMCCK
CEARIKLVVESSADDLRAFQQLFLNTLSFVCPGEPGRTIPYKLPDLCTELNTSLQDIEIT
CVYCKTVLELTEVFEFAFKDGFVVYRDSIPHAACHKLEKLTNTGLYNLLIRCLRCQK
AEKLRHLNEKRRFHNIAGPGDTPTLHEYMLDLQPETTDPDRAHYNIVTFCCKCDSTL
RRCVQSTHVDIRTLEDLLMGTLGIVCPIASQAFQDPQERPRKLPQLCTELQTTIHDIILE
CVYCKQQLLRREVYDFAFRDGCIVYRNPYAVCDKCLKFYSKISEYRHYCYSLYGTTL
EQQYNKPLCDLLIRCINCOKPLRFHNIRGRWTGRCMSCCRS

1. A lentiviral vector, comprising at least four distinct nucleic acid sequences selected from the group consisting of:
at least one nucleic acid sequence encoding a non-oncogenic Human papillomavirus (HPV16) protein E6 antigen,
at least one nucleic acid sequence encoding a non-oncogenic Human papillomavirus (HPV16) protein E7 antigen,
at least one nucleic acid sequence encoding a non-oncogenic Human papillomavirus (HPV18) protein E6 antigen, and
at least one nucleic acid sequence encoding a non-oncogenic Human papillomavirus (HPV18) protein E7 antigen.

2. The lentiviral vector according to claim 1, wherein the nucleic acid sequence encoding the non-oncogenic Human papillomavirus (HPV16) protein E6 antigen encodes an amino acid sequence having at least 80% sequence identity with the amino acid sequence set forth as SEQ ID NO: 7.

3. The lentiviral vector according to claim 1, wherein the nucleic acid sequence encoding the non-oncogenic Human papillomavirus (HPV16) protein E7 antigen encodes an amino acid sequence having at least 68% sequence identity with the amino acid sequence set forth as SEQ ID NO: 16.

4. The lentiviral vector according to claim 1, wherein the nucleic acid sequence encoding the non-oncogenic Human papillomavirus (HPV18) protein E6 antigen encodes an amino acid sequence having at least 60% sequence identity with the amino acid sequence set forth as SEQ ID NO: 24.

5. The lentiviral vector according to claim 1, wherein the nucleic acid sequence encoding the non-oncogenic Human papillomavirus (HPV18) protein E7 antigen encodes an amino acid sequence having at least 83% sequence identity with the amino acid sequence set forth as SEQ ID NO: 33.

6. The lentiviral vector according to claim 1, wherein the at least four distinct nucleic acid sequences encoding antigens are fused together, forming a single antigenic nucleic acid sequence encoding a single antigenic fusion protein under the control of a single promoter sequence.

7. The lentiviral vector according to claim 1, wherein the order of the at least four distinct nucleic acid sequences, from 5′ end to 3′ end, is selected from the group consisting of:
(a) nucleic acid sequence encoding a non-oncogenic Human papillomavirus (HPV16) protein E7 antigen—nucleic acid sequence encoding a non-oncogenic Human papillomavirus (HPV16) protein E6 antigen—nucleic acid sequence encoding a non-oncogenic Human papillomavirus (HPV18) protein E7 antigen—nucleic acid sequence encoding a non-oncogenic Human papillomavirus (HPV18) protein E6 antigen;
(b) nucleic acid sequence encoding a non-oncogenic Human papillomavirus (HPV16) protein E6 antigen—nucleic acid sequence encoding a non-oncogenic Human papillomavirus (HPV18) protein E6 antigen—nucleic acid sequence encoding a non-oncogenic Human papillomavirus (HPV16) protein E7 antigen—nucleic acid sequence encoding a non-oncogenic Human papillomavirus (HPV18) protein E7 antigen;
(c) nucleic acid sequence encoding a non-oncogenic Human papillomavirus (HPV18) protein E6 antigen—nucleic acid sequence encoding a non-oncogenic Human papillomavirus (HPV16) protein E6 antigen—nucleic acid sequence encoding a non-oncogenic Human papillomavirus (HPV18) protein E7 antigen—nucleic acid sequence encoding a non-oncogenic Human papillomavirus (HPV16) protein E7 antigen; and
(d) nucleic acid sequence encoding a non-oncogenic Human papillomavirus (HPV18) protein E7 antigen—nucleic acid sequence encoding a non-oncogenic Human papillomavirus (HPV18) protein E6 antigen—nucleic acid sequence encoding a non-oncogenic Human papillomavirus (HPV16) protein E7 antigen—nucleic acid sequence encoding a non-oncogenic Human papillomavirus (HPV16) protein E6 antigen.

8. The lentiviral vector according to claim 1, wherein the order of the at least four distinct nucleic acid sequences, from 5′ end to 3′ end, is (a) nucleic acid sequence encoding a non-oncogenic Human papillomavirus (HPV16) protein E7 antigen—nucleic acid sequence encoding a non-oncogenic Human papillomavirus (HPV16) protein E6 antigen—nucleic acid sequence encoding a non-oncogenic Human papillomavirus (HPV18) protein E7 antigen—nucleic acid sequence encoding a non-oncogenic Human papillomavirus (HPV18) protein E6 antigen.

9. The lentiviral vector according to claim 1, comprising a nucleic acid sequence which encodes an amino acid sequence having at least 90% sequence identity with the amino acid sequence set forth as SEQ ID NO: 42.

10. The lentiviral vector according to claim 1, being selected from the group consisting of the non-integrative lentiviral vectors filed at the CNCM under accession numbers T-5759, T-5760, T-5761 and I-5762.

11. The lentiviral vector according to claim 1, wherein the lentiviral vector comprises a MHC Class I promoter.

12. The lentiviral vector according to claim 1, wherein the lentiviral vector comprises a cPPT/CTS sequence.

13. The lentiviral vector according to claim 1, wherein the lentiviral vector comprises a 3′ long terminal repeat (LTR) which is devoid of its U3 promoter sequence.

14. The lentiviral vector according to claim 1, wherein the lentiviral vector does not comprise a constitutive enhancer sequence.

15. The non-integrative lentiviral vector according to claim 1, wherein the lentiviral vector comprises a mutant form of the woodchuck hepatitis B virus (WHV) post-transcriptional regulatory element (WPRE).

16. A lentiviral vector particle comprising at least one lentiviral vector as defined in claim 1.

17. The lentiviral vector particle according to claim 16, wherein the lentiviral vector particle comprises a functional lentiviral integrase protein.

18. The lentiviral vector particle according to claim 16, wherein the lentiviral vector particle comprises a vesicular stomatitis virus glycoprotein (VSVG).

19. The lentiviral vector particle according to claim 16, wherein the lentiviral vector particle comprises HIV-1 subtype D Gag and Pol proteins.

20. An isolated cell comprising a lentiviral vector according to claim 1 or lentiviral vector particle comprising the lentiviral vector.

21. A vaccine composition comprising a lentiviral vector according to claim 1, a lentiviral vector particle comprising the lentiviral vector, or a cell comprising either the lentiviral vector or the lentiviral vector particle.

22. A method for the treatment or prevention of an HPV induced cancer in an individual in need thereof, comprising the administration to said individual of a vaccine composition according to claim 21, for use in the treatment or prevention of an HPV induced cancer.

23. (canceled)

24. A method for the treatment or prevention of an HPV induced cancer in an individual in need thereof, comprising the administration to said individual a lentiviral vector according to claim 1, or a lentiviral vector particle comprising the lentiviral vector, or a cell comprising the lentiviral factor or the lentiviral particle.

25. The method of claim 24 wherein administration is performed in combination with at least one immune checkpoint inhibitor.

26. The method of claim 25 wherein the at least one immune checkpoint inhibitor is administered simultaneously or separately.

27. The lentiviral vector according to claim 2, wherein the nucleic acid sequence encoding the non-oncogenic Human papillomavirus (HPV16) protein E6 antigen is selected from the group consisting of SEQ ID NO: 2, SEQ ID NO: 3, SEQ ID NO: 4, SEQ ID NO: 5 and SEQ ID NO: 6.

28. The lentiviral vector according to claim 3, wherein the nucleic acid sequence encoding the non-oncogenic Human papillomavirus (HPV16) protein E7 antigen is selected from the group consisting of SEQ ID NO: 14 and SEQ ID NO: 15.

29. The lentiviral vector according to claim 4, wherein the nucleic acid sequence encoding the non-oncogenic Human papillomavirus (HPV18) protein E6 antigen is selected from the group consisting of SEQ ID NO: 20, SEQ ID NO: 21, SEQ ID NO: 22 and SEQ ID NO: 23.

30. The lentiviral vector according to claim 5, wherein the nucleic acid sequence encoding the non-oncogenic Human papillomavirus (HPV18) protein E7 antigen is selected from the group consisting of SEQ ID NO: 30, SEQ ID NO: 31 and SEQ ID NO: 32.

31. The lentiviral vector according to claim 9, comprising a nucleic acid sequence which is the nucleic acid sequence SEQ ID NO: 41.

33. The method according to claim 22, wherein the HPV induced cancer is selected from the group consisting of cervical cancer, vaginal cancer, vulvar cancer, penile cancer, anal cancer, oropharyngeal cancer, and metastases thereof.

34. The method according to claim 24, wherein the HPV induced cancer is selected from the group consisting of cervical cancer, vaginal cancer, vulvar cancer, penile cancer, anal cancer, oropharyngeal cancer, and metastases thereof.


SEQUENCE LISTING (48 sequences)

SEQ ID NO: 1 - DNA, 477 bp, Human papillomavirus type 16
  misc_feature 1..477: note = nucleic acid sequence encoding the E6 protein from HPV 16(NC_001526.4)
  source 1..477: mol_type = unassigned DNA
atgcaccaaaagagaactgcaatgtttcaggacccacaggagcgacccagaaagttaccacagttatgcacagagctgcaaacaactatacatgatataatattagaatgtgtgtactgcaagcaacagttactgcgacgtgaggtatatgactttgcttttcgggatttatgcatagtatatagagatgggaatccatatgctgtatgtgataaatgtttaaagttttattctaaaattagtgagtatagacattattgttatagtttgtatggaacaacattagaacagcaatacaacaaaccgttgtgtgatttgttaattaggtgtattaactgtcaaaagccactgtgtcctgaagaaaagcaaagacatctggacaaaaagcaaagattccataatataaggggtcggtggaccggtcgatgtatgtcttgttgcagatcatcaagaacacgtagagaaacccagctgtaa

SEQ ID NO: 2 - DNA, 369 bp, synthetic construct
  misc_feature 1..369: note = nucleic acid sequence encoding a non-oncogenic variant of the E6protein from HPV 16
  source 1..369: mol_type = other DNA
gacccccaagaacggcccagaaagctgccccagctgtgcaccgagctgcagaccaccatccacgacatcatcctggaatgcgtgtactgcaagcagcagctgctgagaagagaggtgtacgacttcgccttccgggacctgtgcatcgtgtaccggaacccctacgccgtgtgcgacaagtgcctgaagttctacagcaagatcagcgagtaccggcactactgctacagcctgtacggcaccaccctggaacagcagtacaacaagcccctgtgcgacctgctgatcagatgcatcaactgccagaagcccctgcggttccacaacatccggggcagatggaccggccggtgcatgagctgctgcaga

SEQ ID NO: 3 - DNA, 399 bp, synthetic construct
  misc_feature 1..399: note = nucleic acid sequence encoding a non-oncogenic variant of the E6protein from HPV 16
  source 1..399: mol_type = other DNA
atgggcaccctgggcatcgtgtgccccatcgacccccaagaacggcccagaaagctgccccagctgtgcaccgagctgcagaccaccatccacgacatcatcctggaatgcgtgtactgcaagcagcagctgctgagaagagaggtgtacgacttcgccttccgggacctgtgcatcgtgtaccggaacccctacgccgtgtgcgacaagtgcctgaagttctacagcaagatcagcgagtaccggcactactgctacagcctgtacggcaccaccctggaacagcagtacaacaagcccctgtgcgacctgctgatcagatgcatcaactgccagaagcccctgcggttccacaacatccggggcagatggaccggccggtgcatgagctgctgcaga

SEQ ID NO: 4 - DNA, 372 bp, synthetic construct
  misc_feature 1..372: note = nucleic acid sequence encoding a non-oncogenic variant of the E6protein from HPV 16
  source 1..372: mol_type = other DNA
atggacccccaagaacggcccagaaagctgccccagctgtgcaccgagctgcagaccaccatccacgacatcatcctggaatgcgtgtactgcaagcagcagctgctgagaagagaggtgtacgacttcgccttccgggacctgtgcatcgtgtaccggaacccctacgccgtgtgcgacaagtgcctgaagttctacagcaagatcagcgagtaccggcactactgctacagcctgtacggcaccaccctggaacagcagtacaacaagcccctgtgcgacctgctgatcagatgcatcaactgccagaagcccctgcggttccacaacatccggggcagatggaccggccggtgcatgagctgctgcaga

SEQ ID NO: 5 - DNA, 375 bp, synthetic construct
  misc_feature 1..375: note = nucleic acid sequence encoding a non-oncogenic variant of the E6protein from HPV 16
  source 1..375: mol_type = other DNA
tttcaggacccccaggaaaggcccaggaagttgccccagctctgcaccgaactgcagaccaccattcatgacatcatcctcgaatgcgtgtactgcaagcagcagctcctgaggagggaggtgtacgatttcgccttcagagacggctgtatcgtctacaggaacccctatgccgtctgcgacaaatgcctgaagttttattccaagatctccgagtacaggcactattgctacagcctgtatgggaccaccctggagcagcagtacaacaagcccctgtgcgacctcctgatcaggtgcatcaactgccagaagcccctgaggttccacaacatccgcggcaggtggaccggaaggtgcatgtcctgctgcagg

SEQ ID NO: 6 - DNA, 378 bp, synthetic construct
  misc_feature 1..378: note = the nucleic acid sequence encoding a non-oncogenic variant of theE6 protein from HPV 16
  source 1..378: mol_type = other DNA
ttccaggacccccaggagaggcccaggaaactgccccagttgtgcaccgagctccagacaaccatccacgacatcatcctggagtgcgtgtactgtaagcagcagttgctgaggagagaggtgtatgacttcgccttcagagacggatgcattgtctataggaacccctacgccgtgtgcgacaagtgcctgaagttctactccaagatcagtgagtacaggcattactgctacagcctgtatggaaccacactggaacagcagtacaacaagcccctgtgcgacctcctgattaggtgcatcaactgccagaagcccctcaggttccacaacatccggggcaggtggaccggaaggtgcatgtcctgctgcaggtcc

SEQ ID NO: 7 - protein, 158 aa, Human papillomavirus type 16
  REGION 1..158: note = amino acid sequence of the Wild Type (WT) E6 protein from HPV 16
MHQKRTAMFQDPQERPRKLPQLCTELQTTIHDIILECVYCKQQLLRREVYDFAFRDLCIVYRDGNPYAVCDKCLKFYSKISEYRHYCYSLYGTTLEQQYNKPLCDLLIRCINCQKPLCPEEKQRHLDKKQRFHNIRGRWTGRCMSCCRSSRTRRETQL

SEQ ID NO: 8 - protein, 123 aa, synthetic construct
  REGION 1..123: note = amino acid sequence of a non-oncogenic variant of the E6 proteinfrom HPV 16
DPQERPRKLPQLCTELQTTIHDIILECVYCKQQLLRREVYDFAFRDLCIVYRNPYAVCDKCLKFYSKISEYRHYCYSLYGTTLEQQYNKPLCDLLIRCINCQKPLRFHNIRGRWTGRCMSCCR

SEQ ID NO: 9 - protein, 133 aa, synthetic construct
  REGION 1..133: note = amino acid sequence of a non-oncogenic variant of the E6 proteinfrom HPV 16
MGTLGIVCPIDPQERPRKLPQLCTELQTTIHDIILECVYCKQQLLRREVYDFAFRDLCIVYRNPYAVCDKCLKFYSKISEYRHYCYSLYGTTLEQQYNKPLCDLLIRCINCQKPLRFHNIRGRWTGRCMSCCR

SEQ ID NO: 10 - protein, 124 aa, synthetic construct
  REGION 1..124: note = amino acid sequence of a non-oncogenic variant of the E6 proteinfrom HPV 16
MDPQERPRKLPQLCTELQTTIHDIILECVYCKQQLLRREVYDFAFRDLCIVYRNPYAVCDKCLKFYSKISEYRHYCYSLYGTTLEQQYNKPLCDLLIRCINCQKPLRFHNIRGRWTGRCMSCCR

SEQ ID NO: 11 - protein, 125 aa, synthetic construct
  REGION 1..125: note = amino acid sequence of a non-oncogenic variant of the E6 proteinfrom HPV 16
FQDPQERPRKLPQLCTELQTTIHDIILECVYCKQQLLRREVYDFAFRDGCIVYRNPYAVCDKCLKFYSKISEYRHYCYSLYGTTLEQQYNKPLCDLLIRCINCQKPLRFHNIRGRWTGRCMSCCR

SEQ ID NO: 12 - protein, 126 aa, synthetic construct
  REGION 1..126: note = amino acid sequence of a non-oncogenic variant of the E6 proteinfrom HPV 16
FQDPQERPRKLPQLCTELQTTIHDIILECVYCKQQLLRREVYDFAFRDGCIVYRNPYAVCDKCLKFYSKISEYRHYCYSLYGTTLEQQYNKPLCDLLIRCINCQKPLRFHNIRGRWTGRCMSCCRS

SEQ ID NO: 13 - DNA, 297 bp, Human papillomavirus type 16
  misc_feature 1..297: note = nucleic acid sequence encoding the E7 protein from HPV 16(NP-041326.1)
  source 1..297: mol_type = unassigned DNA
atgcatggagatacacctacattgcatgaatatatgttagatttgcaaccagagacaactgatctctactgttatgagcaattaaatgacagctcagaggaggaggatgaaatagatggtccagctggacaagcagaaccggacagagcccattacaatattgtaaccttttgttgcaagtgtgactctacgcttcggttgtgcgtacaaagcacacacgtagacattcgtactttggaagacctgttaatgggcacactaggaattgtgtgccccatctgttctcagaaaccataa

SEQ ID NO: 14 - DNA, 192 bp, synthetic construct
  misc_feature 1..192: note = nucleic acid sequence encoding a non-oncogenic variant of the E7protein from HPV 16
  source 1..192: mol_type = other DNA
acccccaccctgcacgagtacatgctggacctgcagcccgagacaaccgaccccgaccgggcccactacaatatcgtgaccttctgctgcaagtgcgacagcaccctgcggctgtgcgtgcagagcacccacgtggacatccggaccctggaagatctgctgatgggcaccctgggcatcgtgtgccccatt

SEQ ID NO: 15 - DNA, 201 bp, synthetic construct
  misc_feature 1..201: note = nucleic acid sequence encoding a non-oncogenic variant of the E7protein from HPV 16
  source 1..201: mol_type = other DNA
cccggagacacccccaccctgcacgaatacatgctggacctgcagcccgaaaccaccgaccccgaccgcgctcactacaacatcgttacattctgttgtaaatgcgactccaccctgagaagatgcgtgcagtccacccacgtggacatcaggaccctggaggacctcctcatgggaaccctgggtatcgtctgccccatc

SEQ ID NO: 16 - protein, 98 aa, Human papillomavirus type 16
  REGION 1..98: note = amino acid sequence of the Wild Type (WT) E7 protein from HPV 16
MHGDTPTLHEYMLDLQPETTDLYCYEQLNDSSEEEDEIDGPAGQAEPDRAHYNIVTFCCKCDSTLRLCVQSTHVDIRTLEDLLMGTLGIVCPICSQKP

SEQ ID NO: 17 - protein, 64 aa, synthetic construct
  REGION 1..64: note = amino acid sequence of a non-oncogenic variant of the E7 proteinfrom HPV 16
TPTLHEYMLDLQPETTDPDRAHYNIVTFCCKCDSTLRLCVQSTHVDIRTLEDLLMGTLGIVCPI

SEQ ID NO: 18 - protein, 67 aa, synthetic construct
  REGION 1..67: note = amino acid sequence of a non-oncogenic variant of the E7 proteinfrom HPV 16
PGDTPTLHEYMLDLQPETTDPDRAHYNIVTFCCKCDSTLRRCVQSTHVDIRTLEDLLMGTLGIVCPI

SEQ ID NO: 19 - DNA, 477 bp, Human papillomavirus type 18
  misc_feature 1..477: note = nucleic acid sequence encoding the E6 protein from HPV 18(MF288727.1)
  source 1..477: mol_type = unassigned DNA
atggcgcgctttgaggatccaacacggcgaccctacaagctacctgatctgtgcacggaactgaacacttcactgcaagacatagaaataacctgtgtatattgcaagacagtattggaacttacagaggtatttgaatttgcatttaaagatttatttgtggtgtatagagacagtataccgcatgctgcatgccataaatgtatagatttttattctagaattagagaattaagacattattcagactctgtgtatggagacacattggagaaactaactaacactgggttatacaatttattaataaggtgcctgcggtgccagaaaccgttgaatccagcagaaaaacttagacaccttaatgaaaaacgacgattccacaacatagctgggcactatagaggccagtgccattcgtgctgcaaccgagcacgacaggaaagactccaacgacgcagagaaacacaagtataa

SEQ ID NO: 20 - DNA, 297 bp, synthetic construct
  misc_feature 1..297: note = nucleic acid sequence encoding a non-oncogenic variant of the E6protein from HPV 18
  source 1..297: mol_type = other DNA
ccctacaagctgcctgacctgtgtacagagctgaacacctccctgcaggacatcgagatcacctgtgtgtattgcaagaccgtgctggaactgaccgaggtgttcgagtttgccttcaaggatctgttcgtggtgtaccgggacagcatcccccacgccgcctgccacaagctggaaaagctgaccaacaccggcctgtacaacctgctgattcggtgcctgcggtgtcagaagcctctgaaccccgccgagaagctgcggcacctgaacgagaagcggagattccacaatatcgcc

SEQ ID NO: 21 - DNA, 171 bp, synthetic construct
  misc_feature 1..171: note = nucleic acid sequence encoding a non-oncogenic variant of the E6protein from HPV 18
  source 1..171: mol_type = other DNA
ccctacaagctgcctgacctgtgtacagagctgaacacctccctgcaggacatcgagatcacctgtgtgtattgcaagaccgtgctggaactgaccgaggtgttcgagtttgccttcaaggatctgttcgtggtgtaccgggacagcatcccccacgccgcctgccacaag

SEQ ID NO: 22 - DNA, 297 bp, synthetic construct
  misc_feature 1..297: note = nucleic acid sequence encoding a non-oncogenic variant of the E6protein from HPV 18
  source 1..297: mol_type = other DNA
ccctacaagctgcccgatctgtgcacagagctgaacacctccctgcaggacatcgagatcacctgcgtctactgcaagaccgtgctggaactgaccgaggtgttcgaattcgccttcaaggacggcttcgtggtgtacagggacagcattccccacgccgcctgccataagctggagaaactgaccaacaccggactgtataacctgctgatcaggtgtctgaggtgccagaaggcagagaaactgagacatctgaacgagaaaaggaggttccacaatattgccgggcactgataa

SEQ ID NO: 23 - DNA, 300 bp, synthetic construct
  misc_feature 1..300: note = nucleic acid sequence encoding a non-oncogenic variant of the E6protein from HPV 18
  source 1..300: mol_type = other DNA
atgaggcggccctacaagctgcccgacctgtgcaccgagctgaacacctccctgcaggacatcgagatcacctgcgtgtactgcaagaccgtgctggagctgaccgaggtgttcgaattcgcattcaaggacggattcgtcgtgtatagggacagcattccacacgccgcctgccacaagctggagaaattgactaacaccggactgtataatctgctgatccggtgcctgaggtgtcagaaggccgagaagctgaggcatctgaacgagaaaaggagattccacaatatcgccggacac

SEQ ID NO: 24 - protein, 158 aa, Human papillomavirus type 18
  REGION 1..158: note = amino acid sequence of the Wild Type (WT) E6 protein from HPV 18
MARFEDPTRRPYKLPDLCTELNTSLQDIEITCVYCKTVLELTEVFEFAFKDLFVVYRDSIPHAACHKCIDFYSRIRELRHYSDSVYGDTLEKLTNTGLYNLLIRCLRCQKPLNPAEKLRHLNEKRRFHNIAGHYRGQCHSCCNRARQERLQRRRETQV

SEQ ID NO: 25 - protein, 99 aa, synthetic construct
  REGION 1..99: note = amino acid sequence of a non-oncogenic variant of the E6 proteinfrom HPV 18
PYKLPDLCTELNTSLQDIEITCVYCKTVLELTEVFEFAFKDLFVVYRDSIPHAACHKLEKLTNTGLYNLLIRCLRCQKPLNPAEKLRHLNEKRRFHNIA

SEQ ID NO: 26 - protein, 57 aa, synthetic construct
  REGION 1..57: note = amino acid sequence of a non-oncogenic variant of the E6 proteinfrom HPV 18
PYKLPDLCTELNTSLQDIEITCVYCKTVLELTEVFEFAFKDLFVVYRDSIPHAACHK

SEQ ID NO: 27 - protein, 97 aa, synthetic construct
  REGION 1..97: note = amino acid sequence of a non-oncogenic variant of the E6 proteinfrom HPV 18
PYKLPDLCTELNTSLQDIEITCVYCKTVLELTEVFEFAFKDGFVVYRDSIPHAACHKLEKLTNTGLYNLLIRCLRCQKAEKLRHLNEKRRFHNIAGH

SEQ ID NO: 28 - protein, 100 aa, synthetic construct
  REGION 1..100: note = amino acid sequence of a non-oncogenic variant of the E6 proteinfrom HPV 18
MRRPYKLPDLCTELNTSLQDIEITCVYCKTVLELTEVFEFAFKDGFVVYRDSIPHAACHKLEKLTNTGLYNLLIRCLRCQKAEKLRHLNEKRRFHNIAGH

SEQ ID NO: 29 - DNA, 318 bp, Human papillomavirus type 18
  misc_feature 1..318: note = nucleic acid sequence encoding the E7 protein from HPV 18(NC_001357.1)
  source 1..318: mol_type = unassigned DNA
atgcatggacctaaggcaacattgcaagacattgtattgcatttagagccccaaaatgaaattccggttgaccttctatgtcacgagcaattaagcgactcagaggaagaaaacgatgaaatagatggagttaatcatcaacatttaccagcccgacgagccgaaccacaacgtcacacaatgttgtgtatgtgttgtaagtgtgaagccagaattgagctagtagtagaaagctcagcagacgaccttcgagcattccagcagctgtttctgaacaccctgtcctttgtgtgtccgtggtgtgcatcccagcagtaa

SEQ ID NO: 30 - DNA, 261 bp, synthetic construct
  misc_feature 1..261: note = nucleic acid sequence encoding a non-oncogenic variant of the E7protein from HPV 18
  source 1..261: mol_type = other DNA
aaggccacactgcaggatatcgtgctgcacctggaaccccagaacgagatccccgtggacagcgaggaagagaacgacgagatcgacggcgtgaaccaccagcatctgcccgccagaagggccgagccccagagacacaccatgctgtgcatgtgttgcaaatgcgaggcccggatcaagctggtggtggaaagcagcgccgacgacctgcgggccttccagcagctgttcctgaacaccctgtccttcgtgtgcccttgg

SEQ ID NO: 31 - DNA, 267 bp, synthetic construct
  misc_feature 1..267: note = nucleic acid sequence encoding a non-oncogenic variant of the E7protein from HPV 18
  source 1..267: mol_type = other DNA
ggacctaaagccaccctccaggacatcgtgctgcacctggagccccagaacgagatccccgtcgactcagaggaggagaacgacgaaattgacggcgtcaaccaccagcacctgcccgctcgcagagccgaaccccagagacacaccatgctctgcatgtgctgcaaatgcgaggcccggattaagctggtggtggagagctccgccgacgatctgagagccttccagcagctcttcctgaacaccctgtccttcgtgtgcccctgg

SEQ ID NO: 32 - DNA, 252 bp, synthetic construct
  misc_feature 1..252: note = nucleic acid sequence encoding a non-oncogenic variant of the E7protein from HPV 18
  source 1..252: mol_type = other DNA
ggacctaaagccaccctccaggacatccgtctggagccccagaacgagatccccgtcgactcagaggaggagaacgacgaaattgacggcaaccaccagcacctgcccgctcgcagagccgaaccccagagacacaccatgctctgcatgtgctgcaaatgcgaggcccggattaagctggtggtggagagctccgccgacgatctgagagccttccagcagctcttcctggattccttcgtgtgcccctgg

SEQ ID NO: 33 - protein, 105 aa, Human papillomavirus type 18
  REGION 1..105: note = amino acid sequence of the Wild Type (WT) E7 protein from HPV 18
MHGPKATLQDIVLHLEPQNEIPVDLLCHEQLSDSEEENDEIDGVNHQHLPARRAEPQRHTMLCMCCKCEARIELVVESSADDLRAFQQLFLNTLSFVCPWCASQQ

SEQ ID NO: 34 - protein, 87 aa, synthetic construct
  REGION 1..87: note = amino acid sequence of a non-oncogenic variant of the E7 proteinfrom HPV 18
KATLQDIVLHLEPQNEIPVDSEEENDEIDGVNHQHLPARRAEPQRHTMLCMCCKCEARIKLVVESSADDLRAFQQLFLNTLSFVCPW

SEQ ID NO: 35 - protein, 89 aa, synthetic construct
  REGION 1..89: note = amino acid sequence of a non-oncogenic variant of the E7 proteinfrom HPV 18
GPKATLQDIVLHLEPQNEIPVDSEEENDEIDGVNHQHLPARRAEPQRHTMLCMCCKCEARIKLVVESSADDLRAFQQLFLNTLSFVCPW

SEQ ID NO: 36 - protein, 84 aa, synthetic construct
  REGION 1..84: note = amino acid sequence of a non-oncogenic variant of the E7 proteinfrom HPV 18
GPKATLQDIRLEPQNEIPVDSEEENDEIDGNHQHLPARRAEPQRHTMLCMCCKCEARIKLVVESSADDLRAFQQLFLDSFVCPW

SEQ ID NO: 37 - DNA, 120 bp, synthetic construct
  misc_feature 1..120: note = nucleic acid sequence encoding the cPPT/CTS sequence
  source 1..120: mol_type = other DNA
aattttaaaagaaaaggggggattggggggtacagtgcaggggaaagaatagtagacataatagcaacagacatacaaactaaagaattacaaaaacaaattacaaaaattcaaaatttt

SEQ ID NO: 38 - DNA, 597 bp, synthetic construct
  misc_feature 1..597: note = nucleic acid sequence encoding a mutant form of the woodchuckhepatitis B virus (WHV) post-transcriptional regulatory element(WPRE)
  source 1..597: mol_type = other DNA
ttcccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccgcggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgc

SEQ ID NO: 39 - protein, 15 aa, synthetic construct
ETTDPDRAHYNIVTF

SEQ ID NO: 40 - protein, 15 aa, synthetic construct
PDRAHYNIVTFCCKC

SEQ ID NO: 41 - DNA, 1191 bp, synthetic construct
  source 1..1191: mol_type = other DNA
atgcccggagacacccccaccctgcacgaatacatgctggacctgcagcccgaaaccaccgaccccgaccgcgctcactacaacatcgttacattctgttgtaaatgcgactccaccctgagaagatgcgtgcagtccacccacgtggacatcaggaccctggaggacctcctcatgggaaccctgggtatcgtctgccccatcgcctcccaggcttttcaggacccccaggaaaggcccaggaagttgccccagctctgcaccgaactgcagaccaccattcatgacatcatcctcgaatgcgtgtactgcaagcagcagctcctgaggagggaggtgtacgatttcgccttcagagacggctgtatcgtctacaggaacccctatgccgtctgcgacaaatgcctgaagttttattccaagatctccgagtacaggcactattgctacagcctgtatgggaccaccctggagcagcagtacaacaagcccctgtgcgacctcctgatcaggtgcatcaactgccagaagcccctgaggttccacaacatccgcggcaggtggaccggaaggtgcatgtcctgctgcaggtccgccggccccggacctaaagccaccctccaggacatcgttctccacctggagccccagaacgagatccccgtggactcagaagaggagaacgacgagatcgacggcgtcaaccaccagcacctgcccgctcgcagagccgaaccccagagacacaccatgctctgcatgtgctgcaaatgcgaagcccggattaagttggtggtggaaagcagcgccgacgatctgagggccttccagcagctcttcctcaacaccctgtccttcgtgtgcccctgggtgggcgagcccggtagaaccatcccctacaagctgcccgatctgtgcacagagctgaacacctccctgcaggacatcgagatcacctgcgtctactgcaagaccgtgctggaactgaccgaggtgttcgaattcgccttcaaggacggcttcgtggtgtacagggacagcattccccacgccgcctgccataagctggagaaactgaccaacaccggactgtataacctgctgatcaggtgtctgaggtgccagaaggcagagaaactgagacatctgaacgagaaaaggaggttccacaatattgccgggcactgataa

SEQ ID NO: 42 - protein, 395 aa, synthetic construct
MPGDTPTLHEYMLDLQPETTDPDRAHYNIVTFCCKCDSTLRRCVQSTHVDIRTLEDLLMGTLGIVCPIASQAFQDPQERPRKLPQLCTELQTTIHDIILECVYCKQQLLRREVYDFAFRDGCIVYRNPYAVCDKCLKFYSKISEYRHYCYSLYGTTLEQQYNKPLCDLLIRCINCQKPLRFHNIRGRWTGRCMSCCRSAGPGPKATLQDIVLHLEPQNEIPVDSEEENDEIDGVNHQHLPARRAEPQRHTMLCMCCKCEARIKLVVESSADDLRAFQQLFLNTLSFVCPWVGEPGRTIPYKLPDLCTELNTSLQDIEITCVYCKTVLELTEVFEFAFKDGFVVYRDSIPHAACHKLEKLTNTGLYNLLIRCLRCQKAEKLRHLNEKRRFHNIAGH

SEQ ID NO: 43 - DNA, 1161 bp, synthetic construct
  source 1..1161: mol_type = other DNA
atgttccaggacccccaggagaggccccggaagttgccccagctgtgcaccgagctgcagaccaccatccacgacatcatcctcgaatgcgtgtactgcaagcagcagctgctgaggagggaggtgtatgactttgccttcagagacggatgcattgtctacaggaacccctacgccgtgtgcgacaaatgcctgaagttctactccaagatcagcgagtacaggcactactgctactccctgtacggcaccaccctcgaacagcagtacaacaaacccctgtgcgacctcctgattaggtgcatcaactgccagaagcccctcaggttccacaacatccgcggccgctggaccggccgatgcatgtcttgctgcaggggccccgacgacccctacaagctccccgacctgtgcaccgaactcaacacctccctgcaggacatcgagatcacctgcgtgtattgcaagaccgtgctggagctgaccgaggttttcgaatttgcctttaaggacggcttcgtcgtgtatagggactccatcccccacgccgcctgccataagctggagaagctcaccaacaccggactgtataatctgctgatcaggtgcctcaggtgccagaaggcagaaaagctgaggcatctcaacgagaagcgccggttccacaatattgccggccccggagacacccccacactccatgagtacatgctcgacctgcagcccgaaaccaccgaccccgacagagcccactacaacatcgtgaccttctgctgcaagtgcgactccaccctgagaagatgcgtgcagtccacccacgtggacatccgcacactcgaagacctgctgatgggaaccctgggcatcgtgtgccccatcggccccgatgacaaggccaccttgcaggacatcgtgctgcacctggaaccacagaacgagatccccgtcgactccgaagaagaaaacgacgaaatcgacggagtgaatcaccagcacctgcccgccagaagggccgagcctcagagacacaccatgctctgcatgtgctgcaaatgcgaagccaggattaagctggtggtggagagcagcgccgacgacctgagggccttccagcagctcttcctgaacacactgtccttcgtgtgcccctgggcctgataa

SEQ ID NO: 44 - protein, 385 aa, synthetic construct
MFQDPQERPRKLPQLCTELQTTIHDIILECVYCKQQLLRREVYDFAFRDGCIVYRNPYAVCDKCLKFYSKISEYRHYCYSLYGTTLEQQYNKPLCDLLIRCINCQKPLRFHNIRGRWTGRCMSCCRGPDDPYKLPDLCTELNTSLQDIEITCVYCKTVLELTEVFEFAFKDGFVVYRDSIPHAACHKLEKLTNTGLYNLLIRCLRCQKAEKLRHLNEKRRFHNIAGPGDTPTLHEYMLDLQPETTDPDRAHYNIVTFCCKCDSTLRRCVQSTHVDIRTLEDLLMGTLGIVCPIGPDDKATLQDIVLHLEPQNEIPVDSEEENDEIDGVNHQHLPARRAEPQRHTMLCMCCKCEARIKLVVESSADDLRAFQQLFLNTLSFVCPWA

SEQ ID NO: 45 - DNA, 1170 bp, synthetic construct
  source 1..1170: mol_type = other DNA
atgaggcggccctacaagctgcccgacctgtgcaccgagctgaacacctccctgcaggacatcgagatcacctgcgtgtactgcaagaccgtgctggagctgaccgaggtgttcgaattcgcattcaaggacggattcgtcgtgtatagggacagcattccacacgccgcctgccacaagctggagaaattgactaacaccggactgtataatctgctgatccggtgcctgaggtgtcagaaggccgagaagctgaggcatctgaacgagaaaaggagattccacaatatcgccggacacttccaggacccccaggagaggcccaggaaactgccccagttgtgcaccgagctccagacaaccatccacgacatcatcctggagtgcgtgtactgtaagcagcagttgctgaggagagaggtgtatgacttcgccttcagagacggatgcattgtctataggaacccctacgccgtgtgcgacaagtgcctgaagttctactccaagatcagtgagtacaggcattactgctacagcctgtatggaaccacactggaacagcagtacaacaagcccctgtgcgacctcctgattaggtgcatcaactgccagaagcccctcaggttccacaacatccggggcaggtggaccggaaggtgcatgtcctgctgcaggtccgccggccccggacctaaagccaccctccaggacatcgtgctgcacctggagccccagaacgagatccccgtcgactcagaggaggagaacgacgaaattgacggcgtcaaccaccagcacctgcccgctcgcagagccgaaccccagagacacaccatgctctgcatgtgctgcaaatgcgaggcccggattaagctggtggtggagagctccgccgacgatctgagagccttccagcagctcttcctgaacaccctgtccttcgtgtgcccctgggccggtcccggtgacacacctaccctgcacgagtacatgctcgatctgcagcccgagaccaccgaccccgatcgcgcacactacaacatcgtgaccttctgctgcaaatgtgacagcaccctgagacggtgcgtccagtccacccacgttgacatccgcaccctcgaagacctgctcatgggaaccctgggcatcgtgtgccccatcgcctgataa

SEQ ID NO: 46 - protein, 388 aa, synthetic construct
MRRPYKLPDLCTELNTSLQDIEITCVYCKTVLELTEVFEFAFKDGFVVYRDSIPHAACHKLEKLTNTGLYNLLIRCLRCQKAEKLRHLNEKRRFHNIAGHFQDPQERPRKLPQLCTELQTTIHDIILECVYCKQQLLRREVYDFAFRDGCIVYRNPYAVCDKCLKFYSKISEYRHYCYSLYGTTLEQQYNKPLCDLLIRCINCQKPLRFHNIRGRWTGRCMSCCRSAGPGPKATLQDIVLHLEPQNEIPVDSEEENDEIDGVNHQHLPARRAEPQRHTMLCMCCKCEARIKLVVESSADDLRAFQQLFLNTLSFVCPWAGPGDTPTLHEYMLDLQPETTDPDRAHYNIVTFCCKCDSTLRRCVQSTHVDIRTLEDLLMGTLGIVCPIA

SEQ ID NO: 47 - DNA, 1173 bp, synthetic construct
  source 1..1173: mol_type = other DNA
atgggccctaaggccaccctgcaggacatcgtgctgcacttggagccccagaacgagatccccgtggacagcgaggaggagaacgacgaaatcgacggcgtgaaccaccagcacctgcccgcaagaagggccgaaccccagaggcacaccatgctctgcatgtgctgcaaatgcgaggccaggatcaagctggtggtggaaagcagcgccgacgatctgagggcattccagcagctgttcctgaacaccctctccttcgtgtgccctggggaacccggcaggaccatcccctataaactgcccgacctctgcaccgagctgaacacctccctgcaggacattgagatcacctgcgtctactgcaaaaccgtcctggaactgaccgaggtgttcgagttcgccttcaaagacggcttcgtcgtgtacagggacagcatcccccacgccgcctgccataagctggagaaactgaccaacaccggcctgtacaacctgctgatccggtgcctgagatgtcagaaggccgagaaactgaggcacctcaacgagaaaaggagattccacaatattgccgggcccggcgacaccccaaccctgcacgaatacatgctcgacctgcagcccgaaaccaccgaccccgacagagcccactacaacatcgtgaccttctgctgcaagtgcgactccaccctgagaagatgcgtgcagtccacccacgtggacatccgcacactcgaagacctgctgatgggaaccctgggcatcgtgtgccccatcgcttcccaggcctttcaggacccccaggaacggccaagaaagctgccccagctctgcaccgaactgcagaccaccatccacgacatcatcctggaatgcgtctactgtaagcagcagttgctgaggagggaggtgtatgatttcgccttcagagacggctgcatcgtctacaggaacccctacgccgtgtgcgacaaatgcctgaagttctactccaagatctccgaatacagacactattgctacagcctgtacggcaccaccctcgaacagcagtacaacaaacccctgtgcgacctcctgatcaggtgcatcaactgccagaagcccctccggttccacaacatccgaggaagatggaccggccggtgcatgtcctgctgcaggtcctgataa

SEQ ID NO: 48 - protein, 389 aa, synthetic construct
MGPKATLQDIVLHLEPQNEIPVDSEEENDEIDGVNHQHLPARRAEPQRHTMLCMCCKCEARIKLVVESSADDLRAFQQLFLNTLSFVCPGEPGRTIPYKLPDLCTELNTSLQDIEITCVYCKTVLELTEVFEFAFKDGFVVYRDSIPHAACHKLEKLTNTGLYNLLIRCLRCQKAEKLRHLNEKRRFHNIAGPGDTPTLHEYMLDLQPETTDPDRAHYNIVTFCCKCDSTLRRCVQSTHVDIRTLEDLLMGTLGIVCPIASQAFQDPQERPRKLPQLCTELQTTIHDIILECVYCKQQLLRREVYDFAFRDGCIVYRNPYAVCDKCLKFYSKISEYRHYCYSLYGTTLEQQYNKPLCDLLIRCINCQKPLRFHNIRGRWTGRCMSCCRS